(19) United States
(12) Patent Application Publication
(10) Pub. No.: US 2019/0352377 A1
(43) Pub. Date: Nov. 21, 2019

(54) CHIMERIC ANTIBODIES COMPRISING BINDING DOMAINS OF PHAGE LYSINS, BACTERIAL AUTOLYSINS, BACTERIOCINS, AND PHAGE TAIL OR TAIL FIBERS

(71) Applicant: The Rockefeller University, New York, NY (US)

(72) Inventors: Vincent FISCHETTI, West Hempstead, NY (US); Assaf RAZ, New York, NY (US)

(21) Appl. No.: 16/461,326

(22) PCT Filed: Nov. 15, 2017
(86) PCT No.: PCT/US2017/061684

§ 371 Date: May 15, 2019

Related U.S. Application Data
(60) Provisional application No. 62/422,482, filed on Nov. 15, 2016.

(51) Int. Cl. C07K 16/12; A61P 31/04; C40B 40/10; G01N 33/569; C07K 16/08; C40B 40/02; C12N 15/10; C12N 15/85

(52) U.S. Cl. CPC C07K 16/12; A61P 31/04; C40B 40/10; G01N 33/56911; C07K 16/08; C07K 2317/24; C12N 15/1037; C12N 15/85; C07K 2319/035; C07K 2319/30; C40B 40/02

(57) ABSTRACT

Provided are compositions, methods and kits that are useful for detecting, inhibiting the growth of, and killing bacteria. The compositions include recombinant, chimeric polypeptides that contain at least one immunoglobulin fragment crystallizable region (Fc) segment and at least one additional segment that contains a binding domain that is specific for a bacterial cell wall substrate. The binding domain is one from one or more of a bacterial autolysin, a bacteriophage lysin, a bacteriophage tail or tail fiber, or a bacteriocin. Method of using the polypeptides for detecting, inhibiting the growth of, and killing bacteria are provided and involve contacting bacteria with the polypeptides. Methods of making the polypeptides include expressing the polypeptides in cells, and separating the polypeptides from the cells. Polynucleotides, such as expression vectors, that encode the chimeric polypeptides are also provided.

### CROSS-REFERENCE TO RELATED APPLICATIONS

This application claims priority to U.S. provisional patent application No. 62/422,482, filed Nov. 15, 2016, the disclosure of which is incorporated herein by reference.

### FIELD OF THE DISCLOSURE

The disclosure relates generally to compositions and methods for use in prophylaxis and therapy of bacterial infections. The compositions and methods involve fusion proteins comprising Fc portions of human antibodies and domains from phage lysins and/or bacterial autolysins.

### BACKGROUND ART

The rise of multiple-drug-resistant bacteria has created a clear need for alternatives to conventional antibiotics. One important approach is the use of therapeutic antibodies, which have recently become a mainstay in areas such as cancer therapy and inflammation, and are now increasingly being developed for the treatment of infectious disease. Most antibodies developed thus far target virulence factors that are either secreted or bound to the bacterial surface; however, creation of opsonic antibodies to the carbohydrate components exposed on the surface of bacteria remains an important yet elusive goal of immunotherapy. Carbohydrates are a major component of the Gram-positive bacterial cell wall (up to 60% in dry weight). They are invariant, often surface exposed, and play an important role in wall function. While these properties make surface exposed carbohydrates prime targets for the development of therapeutic antibodies, carbohydrates are poor immunogens. Carbohydrates are T-cell independent antigens, eliciting an immune response characterized by the production of low affinity IgMs, absence of class switched antibodies and memory, and short half-life. While it is possible to promote immunity to certain carbohydrates such as capsular polysaccharides through conjugation to a protein carrier, capsules are often variable and require the production of a polyvalent vaccine for effective protection. Due to these limitations, proteins represent the major class of molecular targets for antibody therapies, and attempts to target carbohydrates have been less successful. Thus, there is an ongoing and unmet need to provide improved compositions and methods that can be used in prophylactic and/or therapeutic approaches to combating pathogenic bacteria. The present disclosure is pertinent to this need.

### SUMMARY

There is an urgent clinical need to create new treatment options to staphylococcal infections. Other than antibiotics, to which staphylococci show resistance, no other anti-infective has been available. Hospitalized patients and those undergoing immunosuppressive therapy are particularly vulnerable, as highly virulent drug resistant bacteria have become endemic in many healthcare facilities. Recent technological advances in the production of recombinant antibodies have made the use of these molecules in the clinic increasingly feasible. Therapeutic antibodies and vaccines are now aggressively being pursued as an alternative treatment for antibiotic resistant bacterial pathogens, as indicated by the number of such agents reaching advanced stages of clinical trials. The approach of the current disclosure demonstrates development of potentially therapeutic antibodies, using binding domains that were optimized through evolution but modified to be components of lysibodies. This approach can be generalizable for many other Gram-positive pathogens, given the wealth of autolysins and phage lysins found in nature Lysibodies therefore, represent a new class of anti-infectives that resolve the long-standing problem of effectively targeting bacterial surface carbohydrates with antibodies. Accordingly, the present disclosure provides compositions and methods for use in prophylaxis and/or therapy of bacterial infections. The disclosure included chimeric polypeptides (referred to herein as “lysibodies” that comprise at least one immunoglobulin fragment crystallizable region (Fc) segment and at least one additional segment that comprises a bacteria binding domain, the bacteria binding domain comprising a binding domain of a bacterial autolysin or a binding domain of a bacteriophage lysin or a binding domain of a bacteriophage tail or tail fiber, or a binding domain of a bacteriocin, or a combination thereof. It should be recognized that the lysibody polypeptides of this disclosure are chimeric polypeptides.

The polypeptides can comprise linkers of varying lengths, such as to separate the Fc and the binding domains, thereby extending the reach of the polypeptides. The polypeptides can comprise more than one Fc region, and can comprise other features, such as protein purification tags.

In various configurations the bacteria binding domain is N-terminal in the polypeptide relative to the Fc segment, or is C-terminal in the polypeptide relative to the Fc segment. The polypeptide(s) may be reversibly or irreversibly attached to substrates. The disclosure includes the polypeptide is in physical association with a molecule on a surface of a bacteria, i.e., a bacteria that is targeted by the polypeptides.

DNA polynucleotide encoding the chimeric polypeptides of this disclosure, and expression vectors comprising the DNA polynucleotides are included. Cells comprising the DNA polynucleotides and expression vectors, as well as their progeny, cell cultures comprising the cells, cell extracts, and the cell culture media are all included.

The disclosure includes methods of inhibiting growth of bacteria and/or killing bacteria or parasites that comprise suitable binding sites that the polypeptides attach to. Bacteria that are resistant to one or more antibiotics can be killed using embodiments of the disclosure. The bacteria may be on or in an individual. Methods for making the polypeptides by allowing expression of the polypeptides in a population of mammalian cells comprising an expression vector encoding the polypeptides, and separating the polypeptides from the population of cells after the expression. Kits and articles of manufacture are provided. In one aspect, the disclosure includes lysibody libraries that comprise a plurality of lysibodies that each have a distinct binding domain specific for a bacterial cell wall receptor. Also provided are methods for treating individuals using personalized approaches by identifying bacteria in an infection and selecting a suitable lysibody from a lysibody library, and treating the individual with the selected lysibody.

### BRIEF DESCRIPTION OF THE FIGURES

FIG. 1—Lysibodies dimerize, form disulfide bridges, and specifically bind their target organism. A. Schematic representations of lysibody structure. B. Structure of the expression vectors for lysibodies and controls. C. Purified lysibodies were run on 10% SDS-PAGE in the presence or absence of the reducing agent β-mercaptoethanol (BME), and analyzed by Western blot using anti-human IgG Fc antibody. A duplicate gel was stained with Coomassie blue. D. Binding of AtlA-lysibody to S. aureus Wood 46 (protein A negative) was determined by deconvolution immunofluorescence microscopy. Maximum intensity projections are presented; anti-human IgG Fc Alexa Fluor 594 conjugate (red), wheat germ agglutinin (green), DAPI (blue). E. Binding of C-terminal Fc fusion lysibodies to S. aureus Wood 46 was determined by immunofluorescence microscopy, using anti-human IgG Fc Alexa Fluor 594 conjugate. Experiments were repeated three times.

FIG. 2—AtlA-Lysibody induces phagocytosis of S. aureus by macrophages. Adherent macrophages were incubated for 1 h with fluorescent S. aureus Newman/pCN57 (GFP) in the presence of various lysibodies. The cells were washed, fixed, and analyzed by microscopy and flow cytometry. A. A representative Raw 264.7 macrophage containing fluorescent staphylococci following ClyS-lysibody treatment; staphylococci—green, wheat germ agglutinin, red—Alexa Fluor 594. The image is presented as a maximum intensity projection; scale bar is 2 μm (also see FIG. 12). B. Gating scheme for flow cytometry analysis: gating on macrophages using forward and side scatter (left), followed by determination of the percentage of highly fluorescent macrophages (right); black—AtlA-lysibody, grey—PBS. C. Effect of lysibody dose on phagocytosis using the N-terminal fusions AtlA-lysibody and ChUb-construct, and 1K8 non-specific humanized monoclonal. D. Effect of lysibody dose on phagocytosis using C-terminal fusions: ClyS-lysibody, PlySs2-lysibody, and PlyG-lysibody. Experiments were performed in triplicates, and repeated three times. E. Percent killing of S. aureus Newman by Raw 264.7 macrophages in the presence of 10 μg of various lysibodies, compared to PBS control. Experiments were performed in triplicates, with two technical repeats for each biological repeat; P values were calculated using t-test.

FIG. 3—Lysibodies induce deposition of complement on the surface of S. aureus cells. S. aureus Wood 46 cells (protein A negative) were attached to poly-L-lysine coated coverslips, and incubated with lysibodies. The cells were then incubated with human complement, washed, fixed, and blocked. Complement was detected using rabbit anti-C3, followed by Alexa Fluor 594 conjugate; DNA was stained with DAPI. Images were obtained using deconvolution microscopy, and are presented as maximum intensity projections. Experiments were repeated twice.

FIG. 4—Lysibodies induce phagocytosis of S. aureus by neutrophils. HL-60 neutrophils (A-D) and human polymorphonuclear cells (E) were incubated with various FITC-labeled S. aureus strains in the presence of lysibodies and S. aureus-adsorbed human complement. A. A representative image of HL-60 neutrophils incubated with FITC-labeled S. aureus USA300 and AtlA-lysibody; a maximum intensity projection is presented, scale bar is 2 μm (also see FIG. 14). B. Gating scheme for flow cytometry analysis: gating on neutrophils using forward and side scatter (left), and determination of the percentage of fluorescent neutrophils (right); black—AtlA-lysibody, grey—PBS. C. Phagocytosis of S. aureus by HL-60 neutrophils using 5 μg lysibody in the presence or absence of complement. D. Effect of lysibody dose on S. aureus phagocytosis by HL-60 neutrophils. E. Phagocytosis of S. aureus by human polymorphonuclear cells using 5 μg lysibody in the presence or absence of complement. All experiments were done in triplicates and repeated two to four times. Statistical significance analysis using the t-test was performed for the relevant samples. P-values are designated as follows: P<0.05 (*), P<0.01 (**), and P<0.001 (***).

FIG. 5—Lysibodies protect mice form MRSA infection in kidney abscess and bacteremia models. A. 5-weeks-old female BALB/C mice were injected with 1 mg of the S. aureus-specific ClyS-lysibody, B. anthracis-specific PlyG-lysibody, or PBS. A day later the mice were injected with 2.5×10⁶ S. aureus USA600 (methicillin resistant, vancomycin intermediate) in 5% mucin. Mouse viability was monitored daily for 4 days, at which time the mice were sacrificed, and the bacterial load per gram in the kidneys was determined through homogenization, serial dilution, and plating. Aggregate data from 4 experiments is presented (n=10 in each group). Statistical significance was determined using two-tailed Mann-Whitney test. B. 5-weeks-old female BALB/C mice were injected with 0.3 mg AtlA-lysibody, or PBS (n=17 in each group). A day later mice were injected with 2×10⁶ S. aureus MW2 (USA400, methicillin resistant) in 5% mucin. Mouse viability was monitored for 8 days. Data represent aggregate results from 4 experiments. Statistical significance was determined using the Gehan-Breslow-Wilcoxon test.

FIG. 6—Structural predictions for lysibody monomers. Protein sequences for the monomeric form of different lysibodies were analyzed by the I-TASSER server. The structures with the highest confidence rate are presented. The human IgG1 Fc region (including hinge) is colored cyan, the binding domain or single chain Fv (where applicable) is colored magenta, the hexahistidine tag is colored yellow, and linker regions are grey.

FIG. 7—S. aureus protein A is saturated in normal human serum—Overnight cultures of S. aureus strains RN4220 (protein A positive) and Wood 46 (protein A negative) were diluted 1:100 in BHI, grown to log phase, and fixed. Cells were attached to poly-L-lysin coated slides and blocked with PBS 1% BSA for 10 minutes. Each slide was further blocked for 1 h with 10 μl human sera, diluted in PBS 1% BSA to the stated concentration. The slides were then incubated with Rhodamine-red-conjugated normal human IgG (non-specific) to test binding to free protein A on the bacterial surface. Scale bar is 2 μm.

FIG. 8—AtlA-lysibody binds to a range of clinically important S. aureus strains. S. aureus strains were fixed and attached to a microscope cover glass. Protein A was blocked with goat and human serum, the bacteria were incubated with Rhodamine-red-conjugated AtlA-lysibody or ChUb-construct, and then visualized by fluorescence microscopy. The brightness level of Mu50 and VRS3a (AtlA-lysibody and ChUb-construct panels) was enhanced to show binding. Scale bar is 2 m.

FIG. 9—ClyS and PlySs2 binding domains specifically binds to a range of clinically important S. aureus strains. Bacterial cells were fixed, attached to a microscope cover glass, and blocked. Bacteria were incubated with purified ClyS-BD GFP fusion, PlySS2-BD GFP fusion, or GFP alone. Slides were imaged by phase-contrast and fluorescence microscopy. Scale bar is 2 m.

FIG. 10—Binding range of AtlA-lysibody. The following strains were evaluated for binding of lysibodies using fluorescence microscopy—S. aureus protein A negative Wood 46, S. epidermidis ATCC 12228S, S. simulans TNK3, S. hyicus HER1048, S. sciuri subsp. sciuri K1, B. cereus T, E. faecalis V12, S. pyogenes SF370, and E. coli DH5α. Bacterial cells were fixed, attached to a microscope cover glass, and blocked. Cells were incubated with AtlA-lysibody or ChUb-construct, and subsequently with anti-human IgG Fc Alexa Fluor 594 conjugate. DNA was visualized using DAPI. Scale bar is 2 μm.

FIG. 11—Binding range of ClyS-lysibody, PlySs2-lysibody and PlyG-lysibody. The following strains were evaluated for binding of lysibodies using fluorescence microscopy—S. aureus protein A negative Wood 46, S. epidermidis ATCC 12228S, S. simulans TNK3, S. hyicus HER1048, S. sciuri subsp. sciuri K1, B. anthracis ΔStrene, B. cereus T, E. faecalis V12, E. faecium EFSK-2, S. pyogenes SF370, and S. agalactiae 090R. Bacterial cells were fixed, attached to a microscope cover glass, and blocked. Cells were incubated with ClyS-lysibody, PlySs2-lysibody or PlyG-lysibody, and subsequently with anti-human IgG Fc Alexa Fluor 594 conjugate.

FIG. 12—High-resolution microscopy of S. aureus Newman/pCN57 (GFP) phagocytosis by Raw 267.4 macrophages. S. aureus Newman/pCN57 (expressing GFP) were added to tissue culture wells containing adherent Raw 264.7 macrophages, supplemented with 5 μg ClyS-lysibody, or 5 μg PlyG lysibody. Following 1 h incubation at 37° C., the wells were washed and macrophages were fixed. Cells were further stained with wheat germ agglutinin Alexa Fluor 594 conjugate, and imaged using deconvolution microscopy. Images are presented as maximum intensity projections. A. Representative images of cells treated with ClyS-lysibody or PlyG-lysibody; scale bar is 5 μm. B. Serial Z-sections at 0.6 μm intervals of a single macrophage containing fluorescent staphylococci treated with ClyS-lysibody; scale bar is 5 μm. C. The number of staphylococci in each macrophage was quantified by analyzing the image stack as presented above. The aggregate results from over 100 macrophages per condition are presented. D. The percentage of intracellular and extracellular bacteria was determined using a similar method; partially phagocytosed bacteria were treated as extracellular.

FIG. 13—AtlA-lysibody induces deposition of complement on the surface of S. aureus cells. A. S. aureus Newman/pCN57 (GFP) cells were attached to a poly-L-lysine coated coverslips, and incubated with AtlA-lysibody or ChUb-construct, and then with S. aureus-adsorbed human complement. The cells were washed, fixed, and blocked (protein A was blocked with heat-inactivated serum). Complement was detected using rabbit anti-C3, followed by Alexa Fluor 594 conjugate. Deconvolution microscopy images are presented as maximum intensity projections. B. S. aureus Newman/pCN57 (GFP) cells were incubated with various concentrations of AtlA-lysibody or ChUb-construct, washed, and incubated with S. aureus-adsorbed human complement. The cells were then washed, fixed, and blocked (protein A was blocked with heat-inactivated serum). Complement was detected using rabbit anti-C3, followed by Alexa Fluor 594 conjugate. For flow cytometry analysis, initial gating was done on GFP-expressing cells, and then the C3 fluorescence in the red channel was evaluated.

FIG. 14—Comparison of the level of phagocytosis as determined by flow cytometry and high-resolution microscopy. HL-60 neutrophils were incubated with FITC-labeled S. aureus strain Wood 46 in the presence of lysibodies and S. aureus-adsorbed human complement. Cells were fixed and each sample was divided for analysis by flow cytometry and deconvolution fluoresce microscopy. For microscopy, the cells were further stained with wheat germ agglutinin Alexa Fluor 594 conjugate. A. Representative images of cells treated with AtlA-lysibody, ChUb-construct, non-specific 1K8 monoclonal, or PBS alone. Images are presented as maximum intensity projections; scale bar is 5 m. B. An example of the technique used to determine whether bacteria are intracellular or extracellular. Z-sections at 0.6 m intervals are presented; intracellular bacteria are denoted with white arrows and extracellular bacteria are denoted with yellow arrows. The scale bar is 5 m. C. At least 150 neutrophils for each treatment group were evaluated by high-resolution fluorescence microscopy for the presence of intracellular and extracellular staphylococci. The results are presented alongside the flow cytometry results obtained from the same sample. D. For each treatment group, the number of intracellular and extracellular bacteria observed by high-resolution fluorescence microscopy per 100 neutrophils is presented.

FIG. 15—Design and production of lysibodies, including bacteriocin binding domains. A. Schematic representation of lysibody structure. B. Structure of the expression vector for lysibodies comprising bacteriocin binding domains. C. Lysibodies were separated by 10% SD S-PAGE and examined by Coomassie blue staining and Western blotting using anti-human IgG horseradish peroxidase conjugate. Samples were loaded in duplicates, either with or without β-mercaptoethanol (BME).

FIG. 16—Lysibodies bind S. aureus. Log-phase S. aureus Wood 46 (protein A negative) were fixed, attached to glass cover slides, and blocked. Binding of lysibodies was determined by immunofluorescence microscopy using anti-human IgG Fc Alexa Fluor 594 conjugate. Scale bar is 5 m.

FIG. 17—Functional characterization of lysibodies. A. ELISA assay performed with S. aureus Wood 46 attached to the bottom of a microtiter well as capture, and varying amounts of lysibody. B. Raw 264.7 macrophages were incubated with S. aureus Newman/pCN57 (GFP) in the presence of lysibodies at different concentration. Percent phagocytosis was determined by flow cytometry. Experiments were done in duplicates. C. HL-60 neutrophils were incubated with FITC-labeled S. aureus Wood 46 in the presence of serially-diluted lysibodies, and 0.5% complement. Experiments were done in triplicates. Percent phagocytosis was determined by flow cytometry. Error bars represent standard deviation.

FIG. 18—Lysostaphin and LysK lysibodies fix complement on the surface of S. aureus. Complement deposition on the surface of S. aureus Wood 46 (protein A negative) was determined by fluorescence microscopy. Staphylococci were attached to cover slides, incubated with lysibodies, and then with S. aureus-adsorbed human complement. The cells were then washed, fixed, and blocked. Complement was detected using specific antibodies and Alexa Fluor 594 conjugate; DNA was stained with DAPI. Slides were imaged using deconvolution microscopy, and images are presented as maximum intensity projections.

FIG. 19—Lysostaphin and LysK lysibodies induce the phagocytosis of S. aureus by macrophages. Raw 264.7 macrophages (A) or peritoneal murine macrophages (B) were incubated with S. aureus Newman/pCN57 (GFP) in the presence of lysibodies at different concentrations. Percent phagocytosis was determined by flow cytometry. Experiments were done in duplicates; the error bars represent standard deviation. (C) Cells of S. aureus strain Newman were incubated with Raw 264.7 macrophages in suspension for 3 hours in the presence of 10 μg lysibodies or controls. Killing compared to PBS control is presented. Experiments were performed in triplicates, with three technical repeats for each biological repeat. Standard deviation values are presented; P values compared to the PlyG-lysibody control were calculated using t-test, ** indicates P<0.01.

FIG. 20—Lysostaphin and LysK lysibodies induce phagocytosis of S. aureus by neutrophils. Neutrophils were incubated for 1 h with FITC-labeled S. aureus strains Wood 46, USA300, and USA600, in the presence of lysibodies, and 0.5% complement unless otherwise noted. Percent phagocytosis was determined by flow cytometry. A. Lysibodies induce the phagocytosis of S. aureus by HL-60 neutrophils in a complement dependent manner; 5 μg lysibody were used per assay. P-values were designated: ** P<0.01, and *** P<0.001. B. Lysibodies induce the phagocytosis of S. aureus by human PMNs in a complement dependent manner; 5 μg lysibody were used per assay. C. Effect of lysibody dose on phagocytosis of S. aureus by HL-60 neutrophils.

FIG. 21—Lysostaphin-lysibody protects mice from MRSA in a kidney abscess model. 5-weeks-old female BALB/C mice were injected with 1 mg Lysostaphin-lysibody or PBS as control. Four hours later, the mice were injected IP with 2.5×10⁶ S. aureus USA600. Mouse viability is presented on panel (A); P value was calculated using log-rank. On the fourth day, surviving mice were sacrificed and the kidneys were removed and homogenized. Bacterial load was determined through serial dilution and plating, and the bacterial load per gram of kidney tissue is presented on panel (B); P value was calculated using Mann-Whitney. Data is aggregated from four separate experiments with a total of 11 mice for Lysostaphin-lysibody and 12 mice for PBS.

FIG. 22—Half-life of Lysostaphin-lysibody in mouse blood. A. Three FVB/NJ female mice were each injected with 1 mg Lysostaphin-lysibody intraperitoneally. Blood was collected by retro-orbital bleeding following 30 min, 1 h, 3 h, and 6 h. Antibody concentration in the serum was determined by capture ELISA. B. Four mice were each injected with 200 μg lysostaphin lysibody, two intra-peritoneally, and two intravenously. Blood was collected following 3 h, 48 h, and 120 h and antibody concentration was determined as above.

FIG. 23—The binding domains of lysostaphin and LysK bind to clinically important strains of S. aureus. Bacteria were fixed and attached to microscope cover glass. The slides were blocked and incubated with lysostaphin-BD-GFP, LysK-BD-GFP, or GFP control. Slides were imaged by fluorescence and phase-contrast microscopy; the scale bar represents 2 μm.

FIG. 24—Relative binding of lysostaphin lysibody to various bacterial species.

FIG. 25—Graph showing percent killing of S. aureus by HL-60 neutrophils following incubation with lysibodies.

FIG. 26—Images showing binding of the S. pyogenes-specific PlyC lysibody to S. pyogenes and S. aureus as determined by fluorescence microscopy.

FIG. 27—Images showing binding of the S. pyogenes-specific spy0077-SH3-lysibody to S. pyogenes as determined by fluorescence microscopy.

FIG. 28—Graph showing induction of phagocytosis of fluorescent S. pneumonia TIGER4 cells into HL-60 neutrophils as determined by flow cytometry with and without complement.

FIG. 29—Summary of data from HL-60 Phagocytosis Assay. A. Flow Cytometry was used to detect neutrophils that have engulfed FITC labeled A Sterne bacteria. Addition of 10 μl of the PlyG lysibody at different concentration results in a slight increase of levels of phagocytosis by HL-60 cells. The amount of lysibody added directly correlates to the levels of phagocytosis by HL-60 cells, the more lysibody added the higher the levels of phagocytosis. B: An example of an HL-60 cell that has phagocytosed and degraded one B. anthracis bacterium and begun digesting a second.

FIG. 30—Graph showing results from a mouse macrophage phagocytosis Assay. Flow Cytometry was used to detect macrophages that have engulfed FITC labeled A Sterne bacteria. Addition of 10 μg of PlyG lysibody results in increased of levels of phagocytosis by mouse macrophages.

FIG. 31—Graph showing results from analysis of B. anthracis lysibody in mice. To obtain the data, vegetative bacteria from the Sterne strain was injected into five mice 3 hour after they were injected with the PlyG lysibody or with PBS control. Mice injected with PlyG lysibody were able to survive longer than mice without.

FIG. 32—Images showing binding of AtlA-lysibody with various mouse Fc regions to S. aureus wood Labeled with secondary antibody α-mouse IgG FITC.

FIG. 33—Graph showing in vitro activity of Mouse IgG2a lysibodies. Induction of phagocytosis of fluorescent S. aureus Wood 46 into HL-60 neutrophils was determined by flow cytometry.

FIG. 34—Graph showing results for HL-60 phagocytosis of S. aureus Wood 46 with lysostaphin mouse IgG. Induction of phagocytosis of fluorescent S. aureus Wood 46 into HL-60 neutrophils was determined by flow cytometry.

FIG. 35—Graphs showing results from analysis of Lysibodies with human IgG1 and IgG3 Fc. Induction of phagocytosis of fluorescent S. aureus into HL-60 neutrophils was determined by flow cytometry.

FIG. 36—Graph showing results from analysis of HL-60 phagocytosis of USA300-FITC. Induction of phagocytosis of fluorescent S. aureus USA300 into HL-60 neutrophils was determined by flow cytometry.

FIG. 37—Photographic and graphic representations of capsule and peptidoglycan, and different Fc configurations.

FIG. 38—Schematics and images for S. aureus specific constructs and linkers. Binding of AtlA-lysibody with various linker regions to S. aureus wood 46 cells was determined by immunofluorescence microscopy.

FIG. 39—Graphs of results obtained using constructs specific for S. aureus specific. Induction of phagocytosis of fluorescent S. aureus into HL-60 neutrophils by lysibodies with different linkers was determined by flow cytometry. Bars in graph are 2 μg, 1 μg, 0.5 μg, and 0.25 μg, shown from left to right in each grouping.

FIG. 40—Graph showing P. pyogenes MD phagocytosis by blood neutrophils. Induction of phagocytosis of fluorescent S. pyogenes M3 into human neutrophils was determined by flow cytometry.

FIG. 41—Schematic and graphical depictions of examples of C-terminal fusion lysibodies with flexible linkers.

FIG. 42—Schematic examples of representative C-terminal fusion lysibodies with halotags (a halotag being a covalent bond between a fusion tag and synthetic ligand) and tropomyosin segments, wherein a tropomyosin functions as a coiled coil molecular rod to extend a spacer between the binding region and the Fc component.

FIG. 43—Graphical depictions of representative C-terminal fusion lysibodies.

FIG. 44—Graphical depictions of additional representative C-terminal fusion lysibodies.

FIG. 45—Images showing binding of PlyC-Fc constructs to S. pyogenes M49.

FIG. 46—Images showing binding of Cpl-1_BD-Fc constructs to S. pneumoniae strain Tigr4. Immunofluorescence images of S. pneumoniae TIGR4, incubated with various lysibodies.

FIG. 47—Schematic representation of lysibody-cytokine fusion proteins, and a bi-specific antibody—lysibody fusion protein (anti mouse CD3—lysostaphin lysibody). Cytokines are fused at the N-terminus. A Lysostaphin binding domain is shown.

FIG. 48—Schematic representation of lysibody-cytokine fusion proteins—cytokines are fused at the C-terminus.

FIG. 49—Images showing Lysibody-cytokine fusion proteins and bi-specific antibody/lysibody fusion proteins retain the ability to bind target bacteria. Determined by fluorescence microscopy.

FIG. 50—Graph showing IFNγ mediated Nitric Oxide production in Raw 264.7 macrophage. AtlA-lysibody mouse interferon gamma fusion protein activate macrophages to produce nitric oxide in the presence of staphylococci. To obtain the results, a monolayer of Raw 264.7 macrophages were supplemented with fusion proteins in the presence or absence of heat killed bacteria and incubated for 24 hours. Alternatively, bacteria were pre-incubated with fusion proteins, washed and added to cells. NO production was determined using Greiss Reagent. Bars in each sample are from left to right: AtlA-Lysibody, AtlA-lysibody mIFNY, AtlA-lysibody-IL-17, mIFNY, and PBS

FIG. 51—Schematic representation of the use of E-tag constructs to opsonize bacteria.

FIG. 52—Schematic representation of E-tag constructs produced for S. aureus.

FIG. 53—Schematic representation of E-tag constructs produced for S. pyogenes.

FIG. 54—Images showing binding of E-tag constructs to S. aureus protein A negative Wood 46 as determined by immunofluorescence.

FIG. 55—Images showing PlyC-Etag mediated complement fixation test on S. pyogenes M55 (fixed). PlyC-Etag (or control) and rabbit anti E-tag were sequentially added to streptococci on a microscope slide and washed. Subsequent deposition of complement from a human serum source was detected using fluorescence microscopy.

FIG. 56—Graphic, and graphs for isolation of S. aureus from a culture using magnetic streptavidin beads and biotin ClyS-B. To obtain the data, Protein A magnetic beads were coated with lysostaphin-lysibody, washed, and rotated with staphylococci at various concentration for 3 h at 4 C. The beads were diluted and plated, final number of bacteria/ml isolated is shown.

FIG. 57—Images from agglutination assay with S. aureus PlySA-NLP-lysibody. In this example a suspension of S. aureus Wood 46 (protein A negative) was mixed with PlySA-NLP-lysibody on a glass slide for 5 minutes at room temperature. Formation of aggregates was visible to the naked eye, and is visualized here using a 10× microscope objective.

FIG. 58—Images from agglutination assay with S. aureus PlySA-NLP-lysibody.

FIG. 59—Graphic, and graphs for Lysibodies with both a catalytic and binding domain. Induction of phagocytosis of fluorescent S. aureus Wood 46 into HL-60 neutrophils was determined by flow cytometry.

FIG. 60—Graphic, and chart for lysibodies with both a catalytic and binding domain. To obtain the data in the chart, PlySs2 lysin, and two catalytic lysibodies were serially diluted (left to right) and added to washed staphylococci. A reduction in optical density indicates lysis of the bacteria. In this experiment PlySs2 catalytic lysibody showed activity that was reduced compared to unmodified PlySs2, but is expected that this can be improved with protein engineering to produce a catalytic molecule with increased half-life.

FIG. 61—Graph showing pharmacokinetics for a representative lysibody. 0.2 mg Lysostaphin lysibody were injected per mouse either intravenously or intraperitoneally. Lysibody concentration in the serum was determined by capture ELISA and the indicated time intervals.

FIG. 62—Graph showing pharmacokinetics for a representative lysibody. 1 mg Lysostaphin lysibody were injected intraperitoneally to each mouse. Lysibody concentration in the serum was determined by capture ELISA at the indicated time intervals.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The present disclosure provides compositions and methods for use in prophylaxis and/or therapy of bacterial infections. Particular embodiments are expected to be suitable for a variety of applications, including but not necessarily limited to treating existing bacterial infections, or for use passively prior to surgery or immunosuppressive treatment to boost immune clearance in hospitalized healthy and immunocompromised patients respectively, and to help control the antibiotic resistant infections that may exist at the time compositions of this disclosure are administered, and/or to help limit opportunistic infections that would otherwise establish infections in immunocompromised individuals.

In non-limiting embodiments the disclosure relates to targeting bacterial surface binding targets. The bacterial surface targets can be targets on or in bacterial walls. The targets can comprise polysaccharide, such as side chains in an LPS of a Gram-negative bacterium, or complexes of teichoic acid covalently linked to a glycan strand in a Gram-positive bacterium, or can comprise pili and flagella and components thereof. It can also comprise the peptidoglycan, its glycan strand or its linked stem peptide. It could also include the cross-bridge or the combination thereof as a conformational receptor. In mycobacteria surface targets can comprise lipids and mycolic acids. Notwithstanding the diversity of bacterial targets that are suitable for targeting with lysibodies of this disclosure, wall carbohydrates may comprise better targets than surface proteins due to their ubiquity in bacterial strains and abundance of epitopes, some of which are invariable. However, the poor immunogenicity of these molecules has made them previously unattractive immunotargets. But the present disclosure takes advantage in part of characteristics that exist in the binding domains of cell wall hydrolases produced by bacteria and bacteriophage. Cell wall hydrolases are enzymes that evolved to recognize wall carbohydrate substrates. In particular, bacteria produce cell wall hydrolases (referred to in the art as “autolysins”) to facilitate peptidoglycan turnover and separate daughter cells following division. These molecules contain binding domains that bind to cell wall carbohydrate substrates and catalytic domains that cleave peptidoglycan bonds. Additionally, bacteriophages also produce cell wall hydrolases (referred to in the art as “lysins”) for releasing progeny phage from infected bacterial hosts. Lysins are two-domain structures, with an N-terminal catalytic domain and a C-terminal binding domain. The present disclosure in various and general embodiments exploits the high affinity binding characteristics of the binding domains of autolysins and lysins to provide chimeric antibody-like molecules, wherein the binding domains of autolysins and lysins replace the fragment antigen-binding (Fab) domain of antibodies. Such constructs are referred to herein as “lysibodies.” Multiple types of lysibodies that incorporate distinct binding domains are demonstrated herein, and are shown to exhibit properties that demonstrate their utility as effective agents to target and kill diverse types of bacteria. Thus, those skilled in the art, given the benefit of the present disclosure, will readily be able to adapt the present teachings to produce and use a wide variety of distinct lysibodies that collectively target, i.e., bind with specificity, diverse different bacterial species and strains. In embodiments a binding domain of a lysibody of this disclosure can comprise phage domains that are part of the phage tail or tail fibers, i.e., all or a segment of a phage receptor binding protein (RBP). These structures can bind to carbohydrates, lipids and proteins. Thus, the disclosure comprises use of bacteria binding domains from autolysins, lysins, bacteriophage tails, bacteriophage tail fibers, and bacteriocins. In embodiments, the RBP is a component of a peptidoglycan hydrolases in a phage tail or tail fiber. The RBP may be at or near the end of the bacteriophage tail or tail fibers, and may be at or near the C-terminal end of the tail or tail fiber. In certain aspects a bacteria binding domain of this disclosure comprises binding domains of autolysins and lysins and phage tail/tail fiber RBPs. Bacteria binding proteins of this disclosure comprising binding domains that bind to a bacterial cell wall substrate.

As discussed above, the lysibodies of this disclosure comprise chimeric antibody-like molecules that have the binding domains of autolysins or lysins or bacteriocins substituted for Fab domain of antibodies. The binding domains are present in fusion proteins that comprise at least one immunoglobulin (Ig) Fc region. In some implementations the Fc region can be of any Ig isotope, but for reasons that will be apparent from the present description, IgG Fc regions are typically preferred. In embodiments, the Ig component comprises an IgG Fc region that is an IgG1, IgG2, IgG3, or IgG4 isotype. Lysibodies may have a portion of an Fab region, or they may be free from Fab segments, and in embodiments can be completely devoid of any portion of an Fab segment. Further, as is known in the art, the H chain constant domain is considered to comprise CH1-CH2-CH3 for IgG, as well as for IgA, IgD, and there is a CH4 domain for IgM and IgE. The CH1 domain is located within the F(ab) region, but the other CH domains (CH2-CH3 or CH2-CH4) comprise the Fc fragment. In certain embodiments, lysibodies of this disclosure comprise an Fc region, and may comprise a CH1 segment of the Fc H chain. In certain embodiments, including CH1 segment provides an additional linker that is relatively stable to proteolysis and thus increases the reach of the lysibody.

In certain embodiments the Fc region comprises only or at least the CH2 and CH3 domains of an IgG heavy chain, and may comprise the hinge region. In an embodiment, the hinge may act as a flexible spacer, and further facilitates formation of disulfide bridges. The disclosure comprises single chain polypeptides, and distinct polypeptides that are covalently linked to one another, such as by a disulfides. The lysibodies may therefore comprise or consist of one or two polypeptide chains, or more polypeptide chains as described more fully below. As one example, the lysibodies can comprise additional Fc regions, and thus the stoichiometry of Fc to binding domains can be altered. In embodiments the lysibodies can comprise two, three, or more Fc segments. In embodiments the Fc segments comprise one or more amino acids that have been altered relative to the naturally occurring Fc amino acid sequences, so long as the function of the lysibody remains adequate for its intended purpose as described herein. Specific and non-limiting examples of Ig mutations are described below.

The Fc region of the lysibodies of this disclosure can comprise or consist of an amino acid sequence that is identical to an Fc region produced by a mammal, such as a human, a mouse or other non-human mammal. In various embodiments, the Fc region can have between 80%-100% (inclusive, and including all numbers there between) amino acid sequence similarity to an Fc region produced by a human or other non-human mammal, such as a mouse. Segments of the Fc region comprise a contiguous Fc region that is adequate to facilitate killing of bacteria to which a lysibody comprising the Fc segment binds.

In embodiments lysibodies are suitable for human or non-human implementations, such as for veterinary purposes. In such examples the Ig Fc segment can optionally be taken from the same animals to which the lysibodies may be administered. In non-limiting embodiments, the lysibodies may be used for companion animals (i.e., canines, felines, equines) and may be used for animals associated with agricultural and food industries (i.e., birds used in the poultry industry, bovine animals used in production of beef and dairy products, and porcine animals, or fish).

Lysibodies of this disclosure can comprise one or more linkers that connect segments of a single fusion protein, or can connect distinct polypeptides. The term “linker” thus refers to a chemical moiety that connects one segment of a polypeptide to another segment of the same polypeptide, or to another polypeptide, or to another agent. Linkers include amino acids, but other linkers are encompassed as well. Generally speaking, amino acid linkers may be principally composed of relatively small, neutral amino acids, such as Glycine, Serine, and Alanine, and can include multiple copies of a sequence enriched in Glycine and Serine. In embodiments the linker has a coiled-coil topology. The coiled-coil topology can be an extended coiled-coil comprised by, for example, a two-stranded alpha-helical coiled coil segment, (for example rabbit skeletal tropomyosin has 259 amino acids per chain of the coiled coil). Multiple copies of the same or distinct linkers can be used in a single fusion protein, and may be connected in series or separated from one another. For example single chain or coiled coil linkers could be 50, 100, 150, 200, 250, 300, 350, 400, 450, 500, amino acids long and anywhere in between.

Lysibodies of this disclosure can be modified to improve certain biological properties, e.g., to improve stability, and/or to enhance certain capabilities, including but not necessarily limited to promoting complement dependent cytotoxicity and/or promoting interaction with phagocytes, such as macrophages and/or neutrophils. Other modifications may involve alteration of a glycosylation pattern, including deletions of one or more glycosylation sites, or addition of one or more glycosylation sites. Lysibodies can be expressed in engineered cell lines with altered glycosylation pathways to result in increased or decreased effector functions Lysibodies may be provided in a composition, in a complex, or covalent linkage with other moieties, including but not necessarily limited to effector molecules such as cytokines. Lysibodies can be conjugated to other agents for other numerous purposes, such as diagnostic applications. Lysibodies can accordingly be modified to be conjugated to detectable labels, including but not limited to visually detectable labels, such as compounds that can fluoresce or emit other detectable signals, such as radiolabels, and to particles for use in separating bacteria, such particles including but not limited to various substrates, including beads made of any material, including but not limited to glass, polymers, and metals, including magnetic beads.

Lysibodies of this disclosure can be made by adapting conventional molecular biology approaches. For example, DNA sequences encoding any lysibody can be constructed based on the coding sequence of any autolysin binding domain and/or any lysin binding domain of interest and any coding sequence of a suitable Ig Fc domain. Thus, the DNA sequences comprise a sequence encoding a fusion protein that contains the autolysin and/or the lysin binding domain and the Fc as a contiguous polypeptide. The resulting DNAs can be placed into any suitable expression vector. The expression vector can include any additional features that may or may not be part of the encoded fusion proteins, such as any suitable promoter, restriction enzyme recognition sites, selectable markers, detectable markers, origins of replication, etc. The vectors can encode leader sequences, purification tags, and hinge segments that separate two or more other segments of the encoded protein. In embodiments, at least one hinge segment separates a binding domain from an Fc region. In an embodiment, a poly-Histidine tag can be used.

The expression vectors can be introduced into any suitable host cells, which can be eukaryotic cells, including but not limited to, simian COS cells, Chinese Hamster Ovary (CHO) cells, human embryonic kidney 293 cells, or any other suitable mammalian cell type such that proper glycosylation of the polypeptide Lysibody occurs. The lysibodies can be expressed and separated from cell cultures that produce them using any suitable reagents and approaches, including but not necessarily limited to protein purification methods that use purification tags, including but not limited to histidine tags, and separating the lysibodies using such tags. Thus, the disclosure includes isolated polynucleotides encoding the lysibodies of this disclosure, cloning intermediates used to make such polynucleotides, expression vectors comprising the polynucleotides that encode the lysibodies, cells and cell cultures that comprise the DNA polynucleotides, cells and cell cultures that express the lysibodies, their progeny, cell culture media and cell lysates that contain the lysibodies, lysibodies that are separated from the cells and are optionally purified to any desirable degree of purity, and compositions comprising one or more lysibodies. The polypeptide may also be purified through the binding of IgG binding proteins such as Protein A or Protein G which bind to the Fc region of the lysibody.

In certain embodiments a method of the disclosure is implemented using an expression vector, such as a plasmid encoding a suitable lysibody to form a type of DNA vaccine. For example, a composition comprising such an expression vector can be administered instead of, or in addition to, the lysibodies themselves. The expression vector would facilitate expression, correct folding, glycosylation and secretion when introduced into mammalian cells in an individual. In an embodiment, cells modified to express a lysibody are introduced into a mammal.

Molecular biology approaches can be adapted to produce lysibodies with multivalent specificities. In embodiments lysibodies of this disclosure can be produced in the form of bivalent lysibodies that comprise two distinct autolysin and/or lysin binding domains. Additional valences are contemplated, including tri-valent constructs. Knob-in-hole approaches can be adapted to produce correctly assembled multi-valent lysibodies that can bind with specificity to two or more distinct bacterial carbohydrate targets.

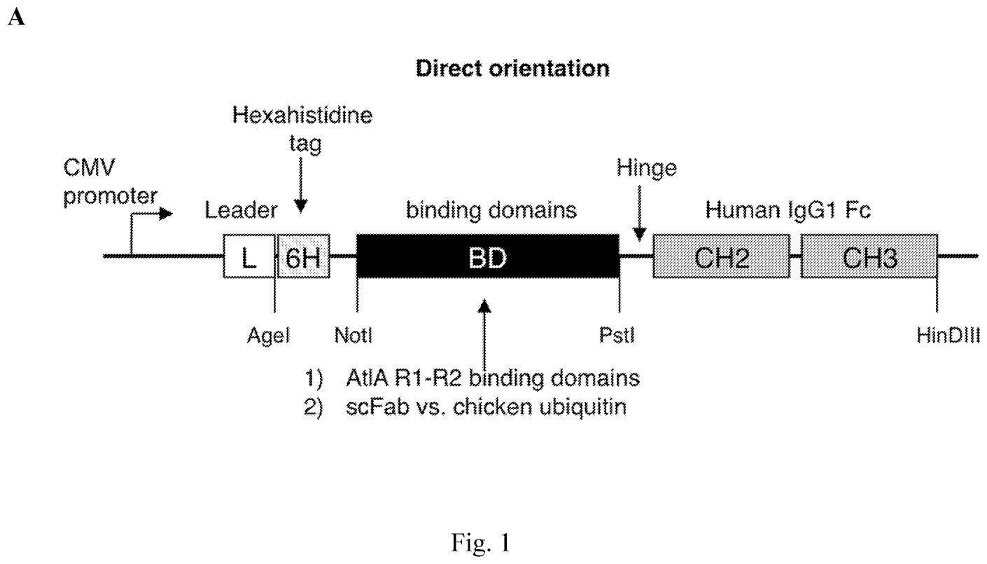

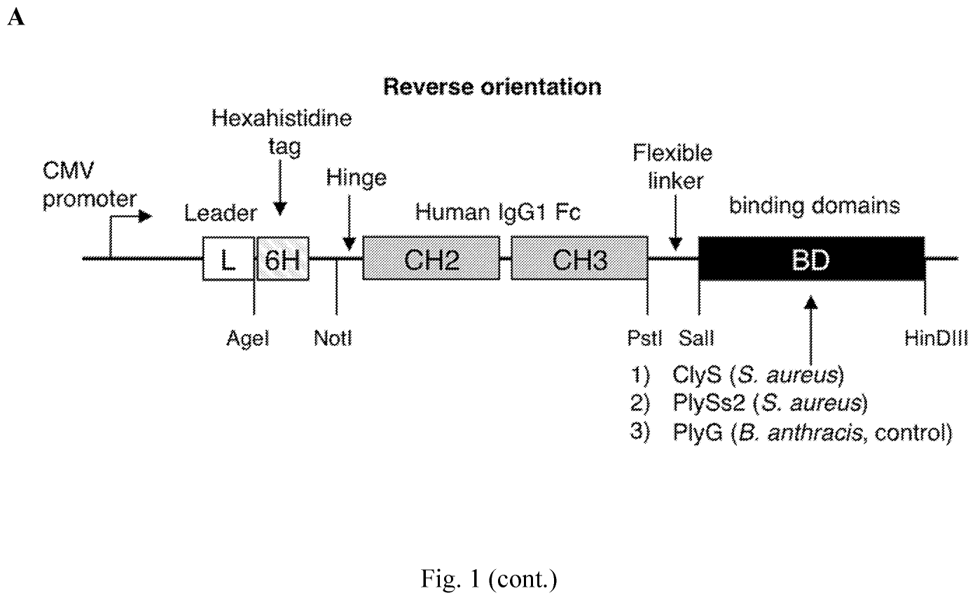

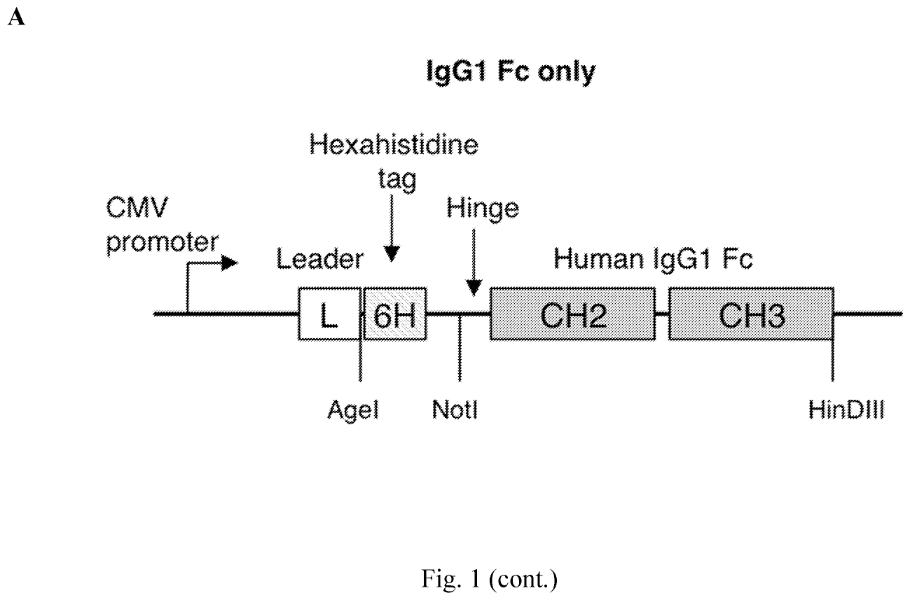

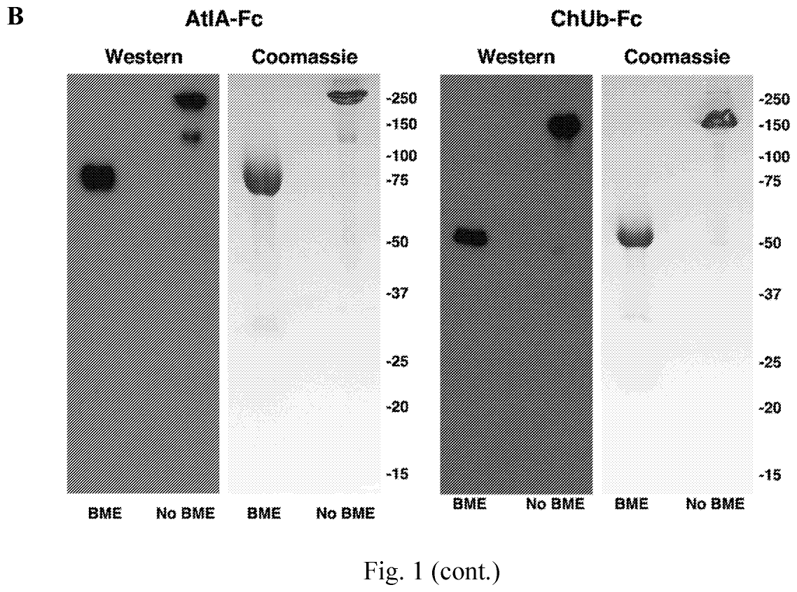

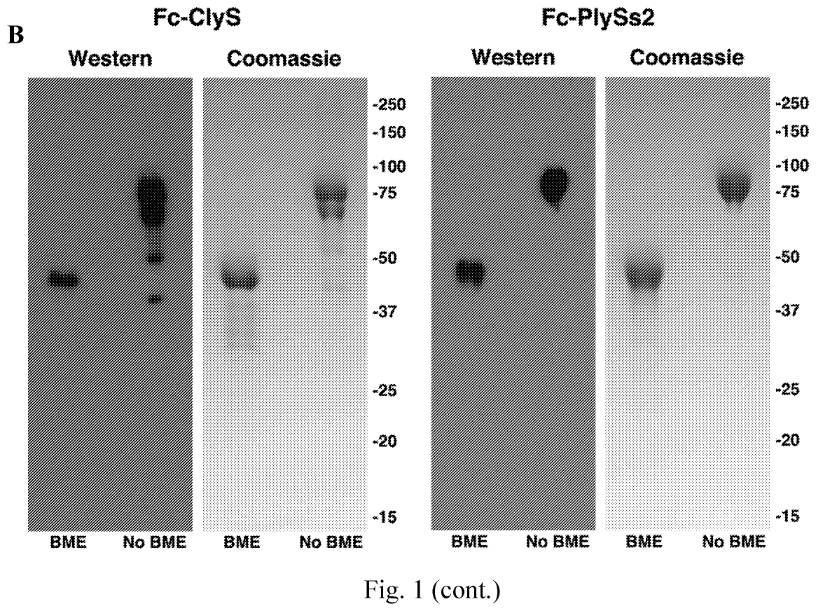

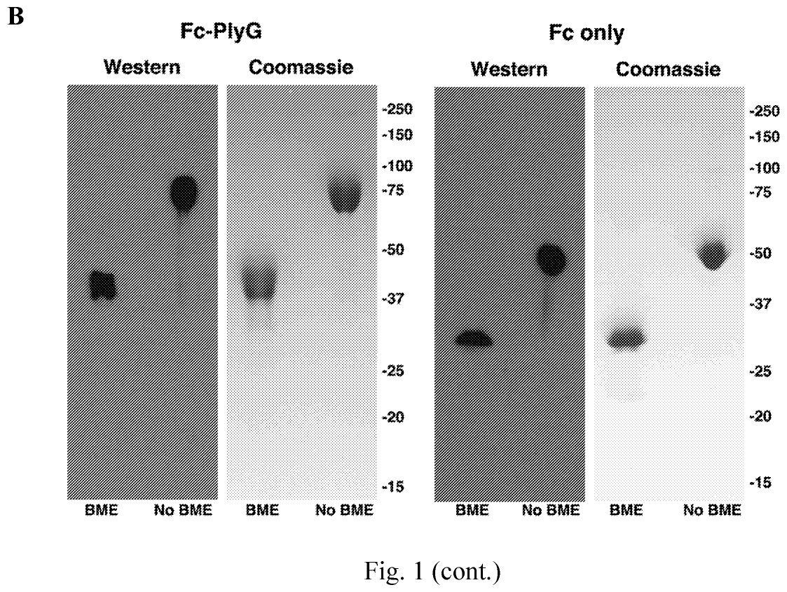

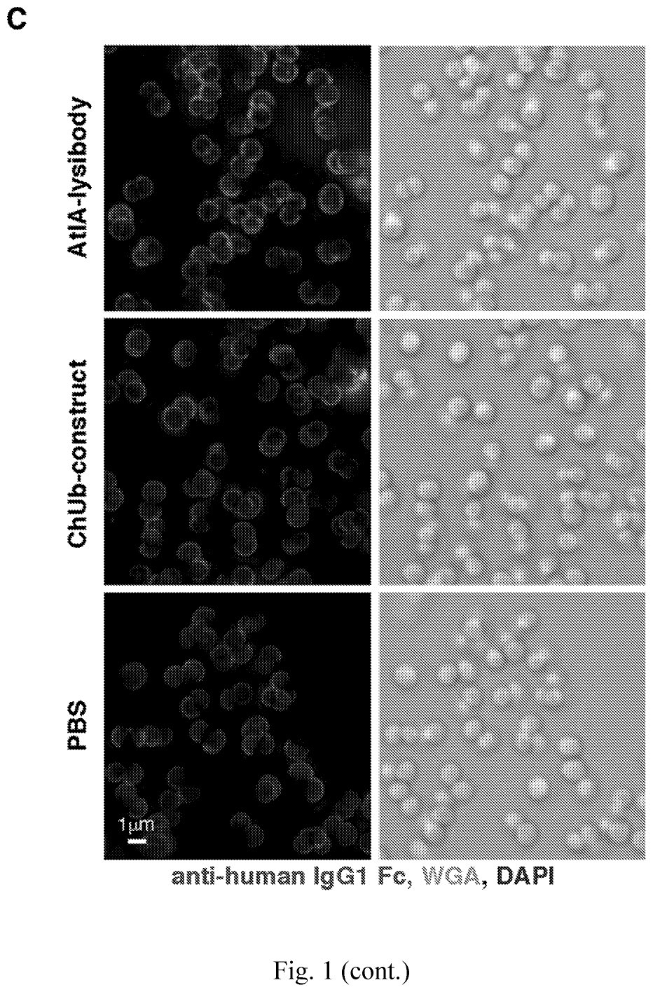

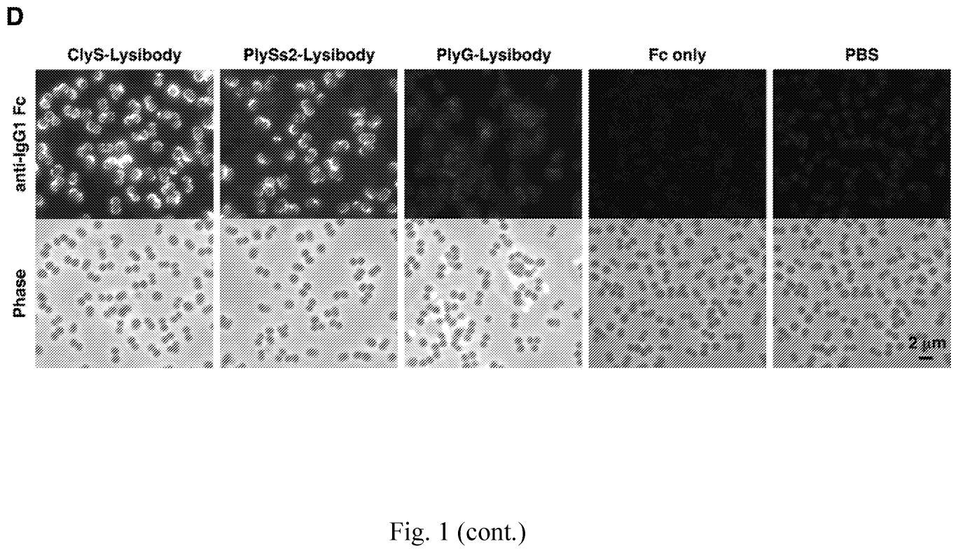

The orientation of the Ig Fc(s) and the binding domain(s) is not limited to a single configuration. The disclosure accordingly encompasses Fc segments that are either at or near the N-terminus or at or the C-terminus of the polypeptide, a vice versa with respect to the binding domains. In non-limiting embodiments a polynucleotide encoding a lysibody, and the lysibody itself, may have either of the general configurations as shown in FIGS. 1A and 1B, which are intended to provide non-limiting illustrations of the relationship between the Ig Fc and the binding domain, with other elements in the figure being optional, as further described herein.

Selection of autolysin binding domains, lysin binding domains binding domains from bacteriophage tails and bacteriophage tail fibers, and bacteriocins is not limited. Thus, lysibodies of this disclosure can be generated using any such binding domain that binds with specificity to targets comprised by pathogenic bacteria. Those skilled in the art will recognize how to test and/or otherwise identify a segment of any autolysin, a lysin, a bacteriocin, a phage tail and a phage tail fiber that is necessary and sufficient to confer adequate binding strength and specificity for use in the methods of this disclosure. In certain embodiments the autolysin binding domain is obtained and/or derived from a gram-positive bacteria. In embodiments, the lysin binding domain is obtained and or/derived from lysins from bacteriophage that infect a Streptococcus, Staphylococcus, Clostridium, Bacillus, Corynebacterium or Listeria. In embodiments the lysin binding domain is obtained and/or derived from any phage that infects gram positive bacteria, and the same can apply to binding domains from bacteriophage tails and bacteriophage tail fibers.

In embodiments, the binding domain is from a bacteriocin, one non-limiting demonstration of which is presented herein using Lysostaphin produced by Staphylococcus simulans biovar staphylolyticus, but other similar bacteriocins and/or binding domains from them can be substituted in lysibodies when given the benefit of the present description. In embodiments, a peptidoglycan or bacteria-specific surface carbohydrate targeting component of a bacteriocin, such as Lysostaphin, is used. Thus, in embodiments, the disclosure includes use of Lysostaphin-like polypeptides in the lysibodies described herein. In connection with this, lysostaphin is made as a proenzyme that comprises three-domains: an N-terminal domain of tandem repeats, a central catalytic domain, and a C-terminal targeting domain. The mature Lysostaphin has the tandem repeats removed, and thus comprises only the catalytic domain and the targeting domain. The C-terminal portion of lysostaphin has 92 amino acids, and is considered to be the targeting domain that directs the interaction of lysostaphin with S. aureus cell walls (see, for example, Baba, et al., Target cell specificity of a bacteriocin molecule: a C-terminal signal directs lysostaphin to the cell wall of Staphylococcus aureus, EMBO J. 1996 Sep. 16; 15(18):4789-97, the description from which is incorporated herein by reference). Thus, in embodiments, a C-terminal cell wall-targeting domain (CWT) of Lysostaphin or a similar bacteriocin is incorporated into a lysibody of this disclosure. In an embodiment, a lysibody of this disclosure comprises a segment from a protein produced by Staphylococcus simulans having the amino acid sequence in GenBank number AAB53783.1, the amino acid sequence of which is incorporated herein as the date it exists in GenBank on the filing date of this application or patent. In embodiments, a domain from a bacteriocin, such as Lysostaphin, is used without an enzymatic domain, such as a glycyl-glycine endopeptidase domain of Lysostaphin. In embodiments, the disclosure includes a segment of ALE-1, which is a close lysostaphin homologue produced by Staphylococcus capitis EPK1 and has a modular structure similar to lysostaphin. It is composed of an N-terminal 13 amino acid repeat domain followed by a central catalytic domain and a C-terminal targeting domain of 92 amino acids that is very similar to the homologous binding domain of lysostaphin.

It will be recognized that incorporating a binding domain from a particular type of bacteria or phage protein into a lysibody confers onto the lysibody specificity for the same type of bacteria, and may confer binding capability and specificity for related bacteria.

Binding domains of lysins typically have a size of about 30 kDa, but the size can vary. Those skilled in the art will recognize that for any particular bacterial autolysin or a bacteriophage lysin the catalytic domain can be readily recognized by sequence similarity with other autolysins and lysins, respectively. For example in a sequence alignment of autolysins, or a sequence alignment of lysins, the catalytic domain will be evident from homology between members of the alignment. Thus, the remaining portion of the sequence comprises a linker and the binding domain. In particular, sequences of peptidoglycan hydrolases in publically accessible databases can be aligned, and such alignments can take into account the class of hydrolase in question, including but not necessarily limited to muramidases, glucosaminidases, amidase, and endopeptidase. Without intending to be bound by theory, in nearly all cases the hydrolase is at the N-terminus of the molecule. Adjacent to this domain is a short (generally 10-20 amino acids) flexible linker, then followed by the binding domain. Accordingly, identification of the catalytic region of an autolysin or a lysin also identifies the binding domain and a linker that separates the binding and catalytic domain. The linker can also be determined using information known to the skilled artisan about linker length and composition, and thus a wide variety of binding domains of lysins and autolysins can be readily identified by those skilled in the art for use in embodiments of this disclosure. Likewise, phage receptor binding domains from bacteriophage tails and bacteriophage tail fibers can be identified by those skilled using various approaches. In certain aspects such binding domains from bacteriophage tails and bacteriophage tail fibers can be determined bioinformatically or otherwise by identifying a catalytic domain of a peptidoglycan hydrolase in the phage genome, which will typically contain the RBP. Additionally or alternatively, phage or components thereof can be labeled and the RBP component identified via binding to bacteria.

The binding domains used in embodiments of this disclosure bind with specificity to certain bacteria. In embodiments, the binding domains have specificity to a ligand on the bacterial cell wall. In embodiments, binding with specificity means the binding domain (and accordingly the lysibody that comprises it) binds exclusively or preferentially to a particular type of target bacteria with higher affinity than to a suitable control or reference, reference, such as bacteria that are not the same species or sub-species as the target bacteria. In embodiments, the lysibodies bind with a higher affinity to the target bacteria relative to affinity for a distinct bacteria species. In embodiments, the ligands to which the binding domains bind with specificity comprise or consist of a protein or a carbohydrate or component thereof that is exposed on the surface of the bacteria.

Representative and non-limiting examples of binding domains that can be incorporated into the chimeric polypeptides of this disclosure include: (“BD”=binding domain):

Lysostaphin BD
(SEQ ID NO: 1)
STAQDPMPFLKSAGYGKAGGTVTPTPNTGWKTNKYGTLYKSESASFTPNT
DIITRTTGPFRSMPQSGVLKAGQTIHYDEVMKQDGHVWVGYTGNSGQRIY
LPVRTWNKSTNTLGVLWGTIK
PlySS2 BD
(SEQ ID NO: 2)
TPPGTVAQSAPNLAGSRSYRETGTMTVTVDALNVRRAPNTSGEIVAVYKR
GESFDYDTVIIDVNGYVWVSYIGGSGKRNYVATGATKDGKRFGNAWGTFK
ClyS BD
(SEQ ID NO: 3)
MNKITNKVKPPNRDGINKDKIVYDRTNINYNMVLQGKSASKITVGSKAPY
NLKWSKGAYFNAKIDGLGATSATRYGDNRTNYRFDVGQAVYAPGTLIYVF
EIIDGWCRIYWNNHNEWIWHERLIVKEVF
AtlA BD
(SEQ ID NO: 4)
TTTPTTPSKPTTPSKPSTGKLTVAANNGVAQIKPTNSGLYTTVYDKTGKA
TNEVQKTFAVSKTATLGNQKFYLVQDYNSGNKFGWVKEGDVVYNTAKSPV
NVNQSYSIKPGTKLYTVPWGTSKQVAGSVSGSGNQTFKASKQQQIDKSIY
LYGSVNGKSGWVSKAYLVDTAKPTPTPTPKPSTPTTNNKLTVSSLNGVAQ
INAKNNGLFTTVYDKTGKPTKEVQKTFAVTKEASLGGNKFYLVKDYNSPT
LIGWVKQGDVIYNNAKSPVNVNIQTYTVKPGTKLYSVPWGTYKQEAGAVS
GTGNQTFKATKQQQIDKSIYLFGTVNGKSGWVSKAYLAVPAAPKKAVAQP
KTA
LysK BD
(SEQ ID NO: 5)
KQIKNYMDKGTSSSTVVKDGKTSSASTPATRPVTGSWKKNQYGTWYKPEN
ATFVNGNQPIVTRIGSPFLNAPVGGNLPAGATIVYDEVCIQAGHIWIGYN
AYNGNRVYCPVRTCQGVPPNQIPGVAWGVFK

The disclosure includes using binding domains that are identical to these amino acid sequences, and thus can comprise or consist of any of these sequences. The disclosure includes amino acid sequences having at least 60, 65, 70, 75, 80, 85, 90, 95, 97, 98, 99 or 99.5% amino acid sequence, inclusive, and including all numbers there between to the first decimal point, identity with these amino acid sequences. The disclosure further includes such polypeptides having the stated amino acid sequence identity, wherein one or more amino acid residues are added, or deleted, wherein such amino acid insertions or deletions may be within the polypeptide, or at the N or C terminus of the sequences, provided the polypeptides maintain specificity for their bacteria surface targets of at least the same specificity/affinity as the sequences given with the sequence identifiers described herein. In embodiments, the polypeptides include one or more conservative amino acid substitutions.

Any lysibody of this disclosure can be modified to reduce its immunogenicity. In embodiments, a binding domain is modified to reduce its immunogenicity, such as by reducing or eliminating T Cell epitopes. In embodiments, the Lysostaphin domain is modified using a known approach or adaptation thereof, such as is described in Zhao et al., 2015, Chemistry & Biology 22, 629-639, the disclosure of which is incorporated herein by reference.

In embodiments, a lysibody of this disclosure exhibits at least one improved property relative to a control. The control can be any suitable value, such as a property determined from a lysibody with a different binding domain than that in the lysibody under consideration. In embodiments, a lysibody of this disclosure has an improved property relative to a control that at least one of improved induction of phagocytosis, improved binding affinity for a bacteria surface ligand, improved inhibition of bacterial growth and/or killing of bacteria, improved protection from the effects of an infection, such as abscess formation, bacteremia, or sepsis, improved reduction in severity of an infection, improved complement fixation, improved agglutination such as in an agglutination assay, an improved pharmacokinetic parameter, an improved half-life or other measure of bioavailability, or any combination of the foregoing.

In embodiments the disclosure relates to reducing the amount of antibiotic resistant and/or virulent bacteria. In embodiments the disclosure relates to killing bacteria that are resistant to a narrow-spectrum beta-lactam antibiotics of the penicillin class of antibiotics. In embodiments, the bacteria are resistant to methicillin (e.g., meticillin or oxacillin), or flucloxacillin, or dicloxacillin, or some or all of these antibiotics. Thus, in one embodiment, disclosure is suitable for killing what has colloquially become known as methicillin-resistant S. aureus (MRSA) which in practice refers to strains of S. aureus that are insensitive or have reduced sensitivity to most or all penicillins. In another embodiment, disclosure is suitable for killing vancomycin resistant bacteria, including but not limited to vancomycin resistant S. aureus (VRSA). In embodiments, vancomycin resistant bacteria may also be resistant to at least one of linezolid (ZYVOX), daptomycin (CUBICIN), and quinupristin/dalfopristin (SYNERCID).

In another aspect the disclosure includes a method for personalized prophylaxis and/or therapy of bacterial infections or diseases. The method comprises obtaining a sample of a bacterial population from an individual in need of prophylaxis and/or therapy for a condition associated with a bacterial infection, and determining the type of bacteria using any suitable approach, such as determining DNA sequences for bacterial species in the sample population. By analyzing the DNA sequences, the presence and/or amount of virulent or otherwise undesirable bacteria can be determined and one or more lysibodies as described herein can be designed or selected from, for example, a pre-existing library of lysibodies that is produced using methods of this disclosure. Such libraries are included in this disclosure. The DNA sequences of the bacteria in the sample can be analyzed using any suitable technique. In this regard, DNA sequencing has been used to identify and catalog many bacteria that make up the human microbiota. Further, many sequencing approaches, such as so-called deep sequencing, massively parallel sequencing and next generation sequencing can be used and such services are offered commercially by a number of vendors. Once the presence/identity of pathogenic bacteria from the sample from the individual is determined a composition comprising one or more suitable lysibodies is administered to the individual such that at least some of pathogenic bacteria are killed.

In certain embodiments lysibodies of this disclosure are provided as components of compositions that comprise a pharmaceutically acceptable carrier. The term “pharmaceutically acceptable carrier” as used herein refers to a substantially non-toxic carrier for administration of pharmaceuticals in which the compound will remain stable and bioavailable. Combining a pharmaceutically acceptable carrier in a composition with a lysibody yields “pharmaceutical compositions.” Some suitable examples of pharmaceutically acceptable carriers, as well as excipients and stabilizers can be found in Remington: The Science and Practice of Pharmacy (2005) 21st Edition, Philadelphia, Pa. Lippincott Williams & Wilkins.

Methods for administering compositions comprise parenteral, intraperitoneal, intrapulmonary, oral, mucosally, and topical administrations. Parenteral infusions include intramuscular, intravenous, intraarterial, intraperitoneal, and subcutaneous administration. The amount of the lysibodies and any other active agent to be included in a composition and/or to be used in the method can be determined by those skilled in the art, given the benefit of the present disclosure. Thus, in one embodiment, an effective amount of a composition of the invention is administered. An effective amount can be an amount that that alleviates disease symptoms associated with a bacterial infection, or reduces bacteria, or eradicates bacteria. An effective amount can vary depending on pharmaceutical formulation methods, administration methods, the patient's age, body weight, sex, type and location of bacterial infection, diet, administration time, administration route, and other factors that will be apparent to those skilled in the art. Compositions can be administered once, or over a series of administrations. Those skilled in the art will be able to determine or predict the half-life of any particular lysibody, which can affect administration. In embodiments, the disclosure includes a single dose, or several doses over the course of a bacterial infection, and typically over a period of about 2-3 weeks. In embodiments, an amount of lysibody from 1 microgram/kg to 1000 milligrams/kg, or higher amounts, are administered as necessary. Dosing can also take into account the specific activity of the lysibody.

In certain embodiments a composition comprising lysibodies is administered to an individual in need thereof. The individual can be diagnosed with, suspected of having, or be at risk for contracting a bacterial infection. In embodiments, the individual is in need of treatment for or is at risk of contracting a nosocomial infection. In embodiments, the individual could be a military/first responder personnel entering a location that is known or is suspected to be contaminated with pathogenic bacteria. In embodiments, the individual is an immunocompromised individual. In embodiments, a composition of the disclosure is applied to a wound. As such, the compositions can be provided with or on bandages, wound dressings, sutures, and the like. In embodiments a composition of this invention is used for treatment and/or prophylaxis of a sexually transmitted bacterial disease, and as such can be formulated for intravaginal administration, and/or for use with prophylactic devices. In embodiments compositions comprising lysibodies could be used for coatings of, for example, medical implantable medical devices, and in such situations (which are not exclusive of other situations) may be detectably labelled. In embodiments, lysibodies are non-covalently or covalently attached to a substrate. In embodiments one or more lysibodies can be attached to a substrate and used in various diagnostic approaches to determine the presence, absence, type and/or amount of bacteria.

In certain aspects, the disclosure provides a bacterium or population of bacteria that are in physical association with lysibodies of this disclosure. Thus, bacteria that have been opsonized by lysibodies of this disclosure are encompassed. In certain embodiments the disclosure comprises a population of bacteria, wherein the bacterial cells comprise a lysibody of this disclosure in physical association with a carbohydrate present on the surface of the bacteria. In embodiments, the disclosure comprises a mixed population of bacterial cells that comprise a lysibody of this disclosure in physical association with a carbohydrate present on the surface of the bacteria, wherein the bacterial population further comprises eukaryotic cells, such as phagocytes, including but not limited to macrophages and neutrophils. In embodiments, the disclosure comprises macrophages and/or neutrophils that have phagocytized one or more bacterial cells that comprise a lysibody of this disclosure in physical association with a carbohydrate present on the surface of the bacteria.

In embodiments the disclosure comprises lysibodies that have been reversibly or irreversibly attached to a substrate. The lysibodies that have been reversibly or irreversibly attached to a substrate may be in physical association with bacteria. The substrate may be a component of a diagnostic device. In embodiments, the lysibodies are used in an immunodiagnostic method and/or device. Thus, in certain aspects the invention provides for detecting the presence or absence of bacteria using any of a variety of approaches for detecting proteins that include lysibodies as detection agents, such as immunodetection methods, including but not limited to Western blotting, multi-well assay plates adapted for detection of proteins, beads adapted for detection of proteins, a lateral flow device or strip that is adapted for detection of proteins, ELISA assays, or any other modification of an immunodetection or other assay type that is suitable for detecting proteins. Those skilled in the art will recognize that, given the benefit of the present disclosure, these and other detection methods can include use of one or more lysibodies as binding partners in diagnostic detection assays. In various embodiments, the one or more lysibody binding partners can be reversibly or irreversibly attached to a substrate, such as by being covalently, ionically, or physically bound to a solid-phase immunoabsorbent using methods such as covalent bonding via an amide or ester linkage, ionic attraction, or by adsorption. The substrate can be any suitable substrate onto which a lysibody binding partner can be attached. Examples include substrates typically used in immunodetection assays, lateral flow devices, bead-based assays, microfluidic devices, etc. Thus, the solid substrate can be a porous solid substrate that allows the flow of liquid through the substrate. The liquid can flow through the porous substrate via any suitable means, such as by capillary action, microfluidics, etc. The substrate can also be a non-porous solid substrate, such as beads formed from glass or other non-porous materials. The immune assay can include any form of direct detection, or any form of ELISA assay. Compositions comprising intact antibodies bound to lysibodies are also included within the scope of this disclosure. In embodiments, the disclosure comprises an article of manufacture comprising packaging and at least one container, the container comprising a pharmaceutical composition comprising one or more lysibodies, and pharmaceutically acceptable salts thereof, the packaging comprising printed information, the printed information providing an indication that the pharmaceutical composition is for use in prophylaxis and/or treatment of bacterial infections, and/or for killing bacteria.

Additional description and data are provided in the Examples of this disclosure.

To provide context for the Examples, which demonstrate particular and non-limiting implementations of the invention, it is notable that previous attempts to target bacteria wall carbohydrates have been largely unsuccessful. And while high affinity antibodies to wall carbohydrates are rare, these wall substrates are bound with high affinity by a variety of cell wall hydrolases, which are ubiquitous in nature. In this regard, the rise in antibiotic resistance is a major concern, which is not adequately addressed by the anti-infective development pipeline. In particular, MRSA is now prevalent in both the hospital and community settings, representing an enormous public health burden worldwide. Vaccines and therapeutic antibodies represent a prominent alternative to antibiotics, however to date none has successfully reached approval for clinical use. Wall carbohydrates may provide a superior approach, given that they are highly conserved among staphylococci, and are a dominant feature of the staphylococcal surface.

The following Examples are intended to illustrate but not limit the invention.

### Example 1

### Lysibody Construction and Production

IgG antibodies are composed of two heavy chains and two light chains, stabilized by disulfide bridges and non-covalent interactions. Each antibody can be functionally divided into two Fab fragments, which bind to target epitopes, a hinge domain, and an Fc fragment, which through its ability to bind to a diversity of Fc receptors, including FcRn and Type I and II FcγRs, determines half-life, and mediates effector functions that lead to the elimination of pathogens. Lysibodies were produced by fusing a human IgG1 Fc with a cell wall binding domain of a bacterial or bacteriophage origin. The general design of lysibodies is presented in FIG. 1A. Lysibodies contain a leader sequence to promote secretion, and a hexahistidine tag for purification. Cysteine 220 of human IgG1, which in the native molecule forms a disulfide bridge with the light chain, was mutated to glycine since a light chain is not present in lysibodies. Thus, the final structure is a two-chain, single domain antibody. Lysibodies were produced in mammalian cells to allow proper glycosylation of the Fc fragment, required for effector functions.

The AtlA-lysibody was created by replacing the V_{H} and C_{H1} domains of human IgG1 heavy chain with the R1-R2 binding repeats of the major staphylococcal autolysin AtlA. AtlA binds S. aureus lipoteichoic acids (LTA), which are essential for S. aureus survival. AtlA binding-repeats R1-R2 bind the S. aureus wall with high affinity, and have approximately 10⁸ binding sites per cell, which are located predominantly in the vicinity of the division rings. As control, we produced a construct, in which the AtlA binding repeats were replaced with a single chain Fv specific for chicken ubiquitin, termed “ChUb-construct” (FIG. 1A).

A slightly different approach was used to produce lysibodies containing binding domains from phage lysins. Unlike autolysins, the cell wall binding domain of most phage lysins is found at the C-terminus of the molecule. Placing such domains at the N-terminus of an antibody Fc region (directly replacing the Fab) results in an unnatural orientation for the lysin binding domain, interfering with its function. As there are numerous examples of functional antibodies with C-terminal Fc fusions, we created lysibodies using the phage lysin binding domains of ClyS and PlySs2 fused to the C-terminus of the human IgG1 antibody Fc portion (FIG. 1A). As controls we also produced a lysibody containing the binding domain of the phage lysin PlyG, which is specific for Bacillus anthracis, and a similar construct that lacks a binding domain altogether (FIG. 1A). We used the I-TASSER server to perform a structural prediction analysis for the monomeric form of all lysibodies. This analysis showed an extended structure with clear domain delineation for lysibodies, resembling that of the control ChUb-construct, which has a scFv as a binding domain (FIG. 6). These structures would likely be further stabilized through dimerization of the IgG Fc portion of the molecule.

Lysibodies were produced by 293T mammalian cells, with over 90% of the protein secreted, as determined by Western blot. Like typical single domain antibodies, lysibodies formed the expected dimers that were stabilized by disulfide bridges, as determined by SDS-PAGE and Western blot analysis (FIG. 1B). Elimination of disulfide bridges with β-mercaptoethanol (BME) resulted in bands at half the molecular weight of the untreated molecule. Dimerization likely increases the binding avidity of lysibodies compared to the original lysin or autolysin.

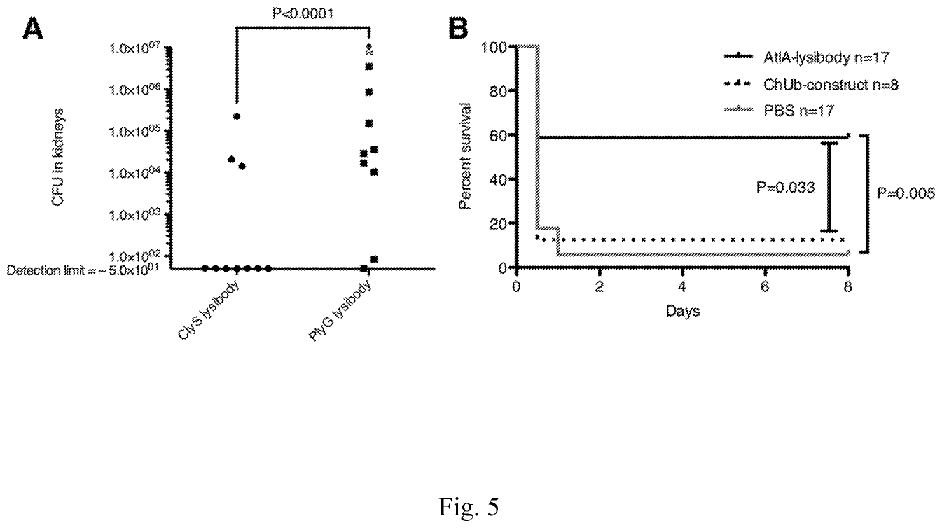

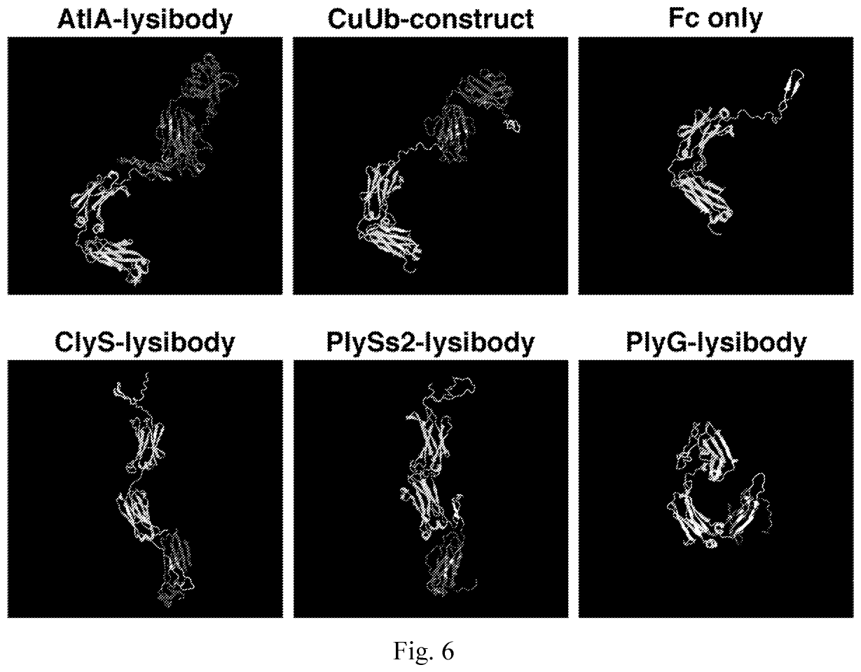

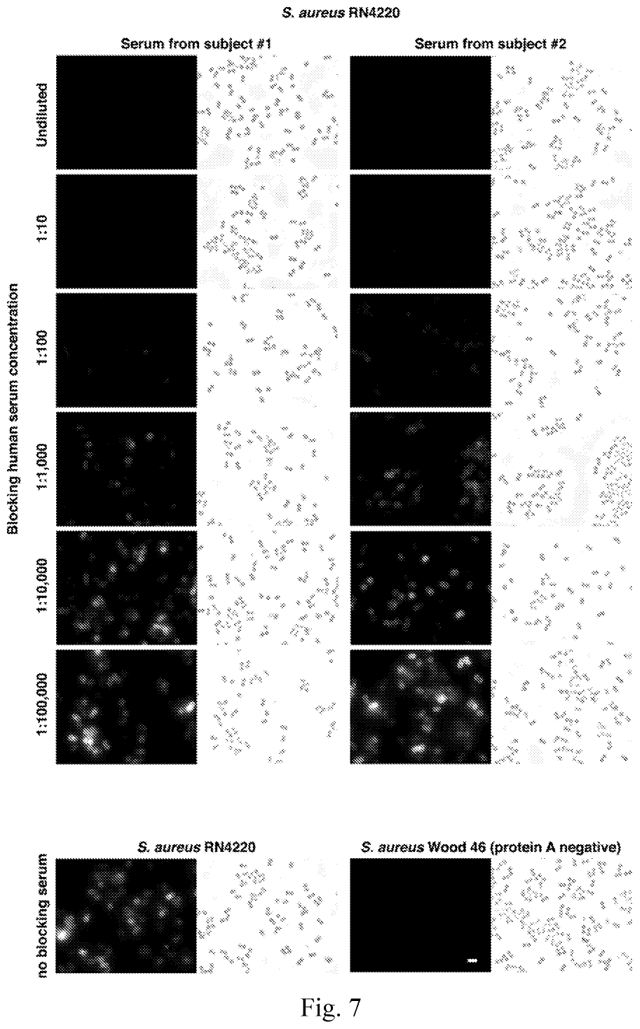

We used fluorescence microscopy to test the binding of lysibodies to the wall of S. aureus. Initially, we used the protein A negative S. aureus strain Wood 46, to avoid non-specific binding of protein A to the Fc region of lysibodies. While the use of a protein A negative strain is one way to addresses this technical issue in microscopy studies in vitro, the abundance of non-specific IgGs found in human serum would likely saturate protein A in vivo (FIG. 7), and thus make it irrelevant for the activity of lysibodies. AtlA-lysibody showed extensive labeling of the staphylococcal cell wall with some preference for the septa (FIG. 1C), similar to previous observations made with a GFP-tagged AtlA-binding domain. No signal was detected with the ChUb-construct or the PBS control (FIG. 1C). Similarly, C-terminal fusion ClyS and PlySs2 lysibodies bound S. aureus, while the anthrax-specific PlyG-lysibody and the Fc-only construct showed little to no binding (FIG. 1D).

### Example 2

### Target Binding Range of Lysibodies

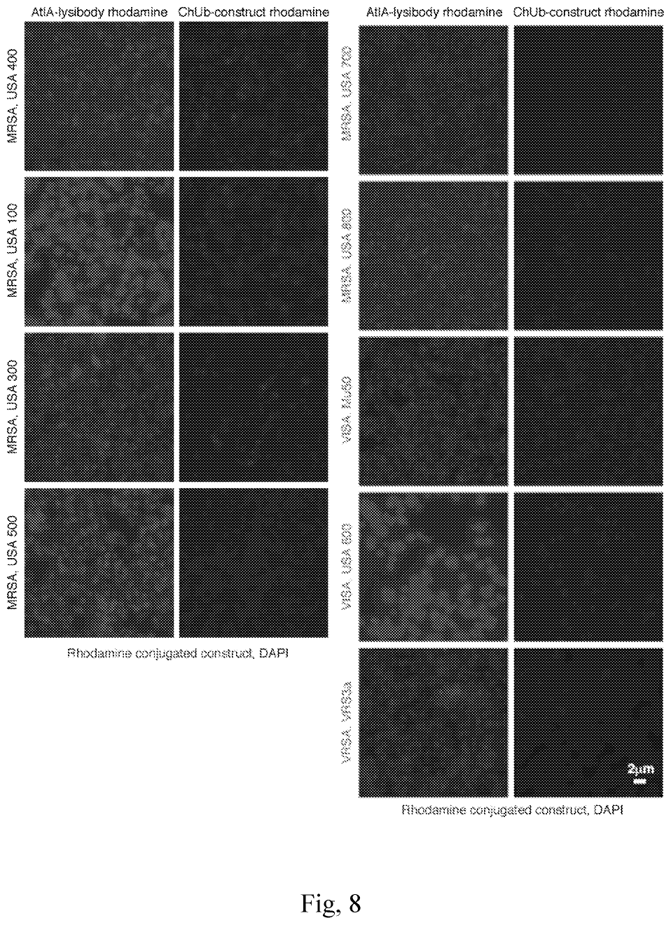

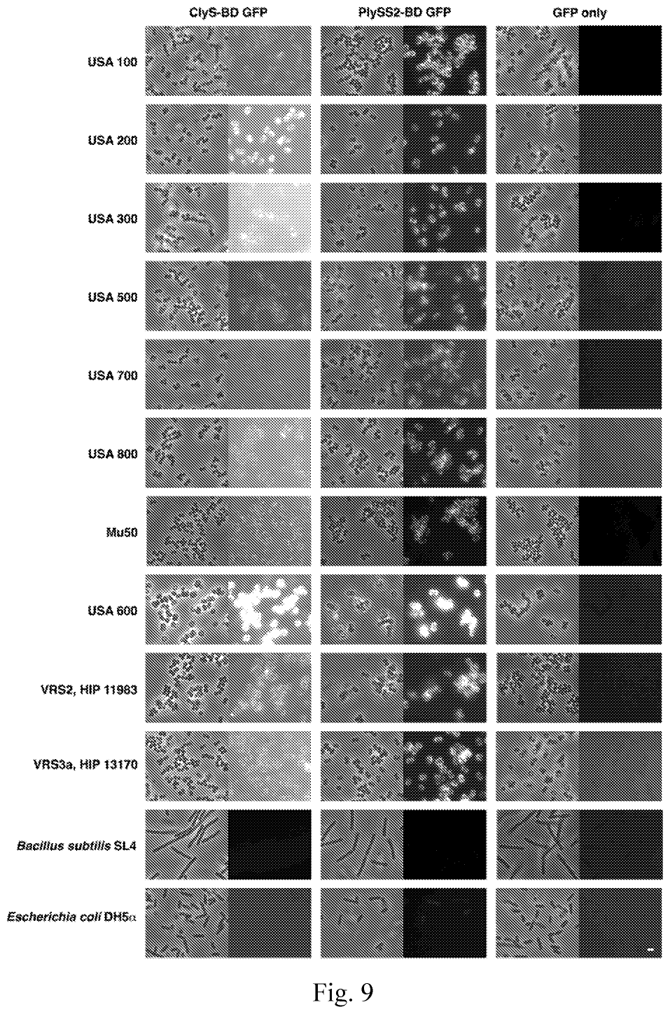

We evaluated the binding of lysibodies to various methicillin resistant, vancomycin intermediate, and vancomycin resistant S. aureus strains (MRSA, VISA, and VRSA respectively) using fluorescence microscopy. To avoid possible non-specific interaction of the Fc portion of lysibodies with staphylococcal protein A, we first blocked protein A with goat and human serum, and used AtlA-lysibody or ChUb-construct that were directly labeled with Rhodamine red. To determine the binding range of ClyS and PlySs2, we used fusion proteins of green fluorescent protein (GFP) and the lysin binding domain alone. The binding domains of AtlA, ClyS, and PlySs2 bound all S. aureus clinical isolates tested, although some variability in fluorescent signal was observed. Controls did not bind any of the strains tested (FIGS. 8, 9).

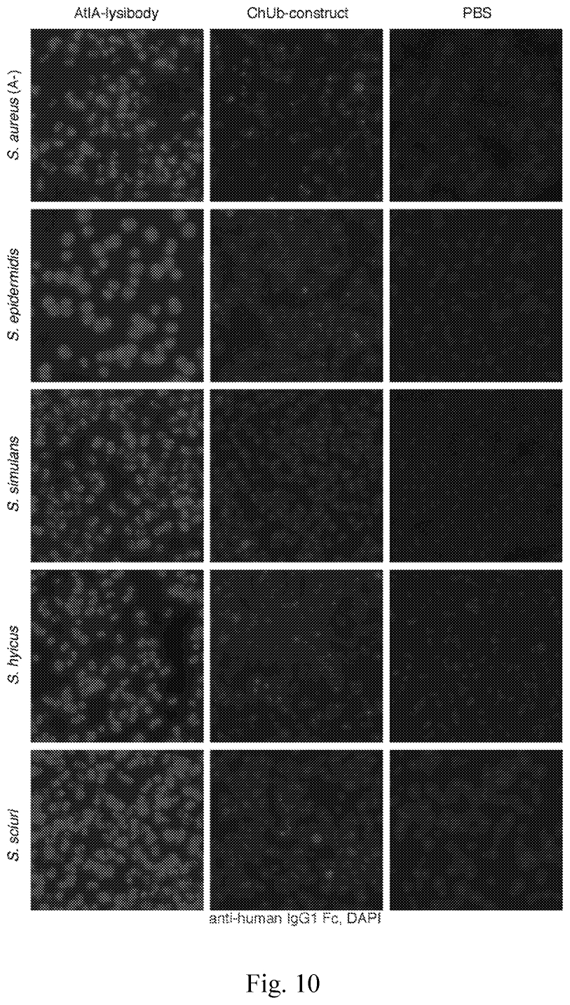

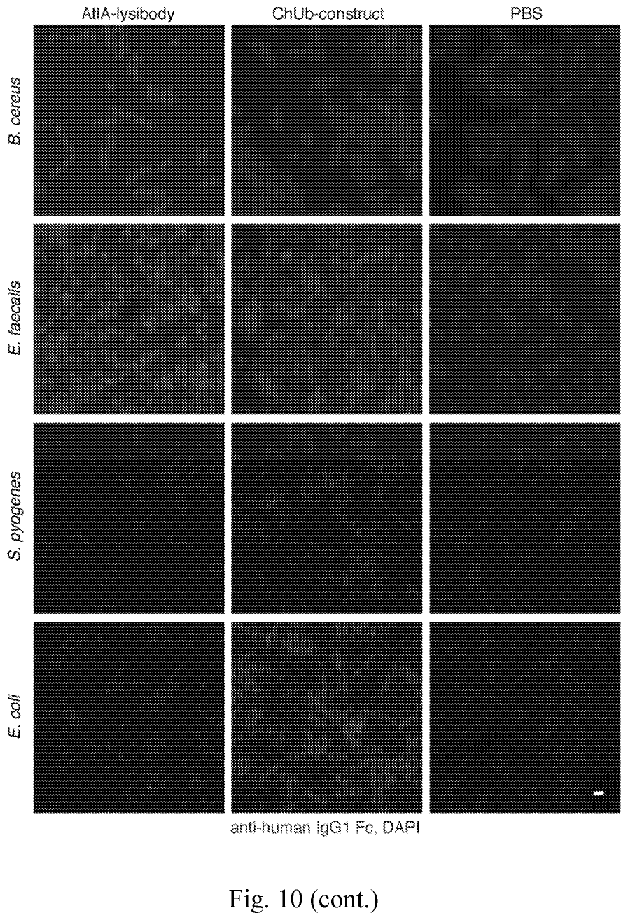

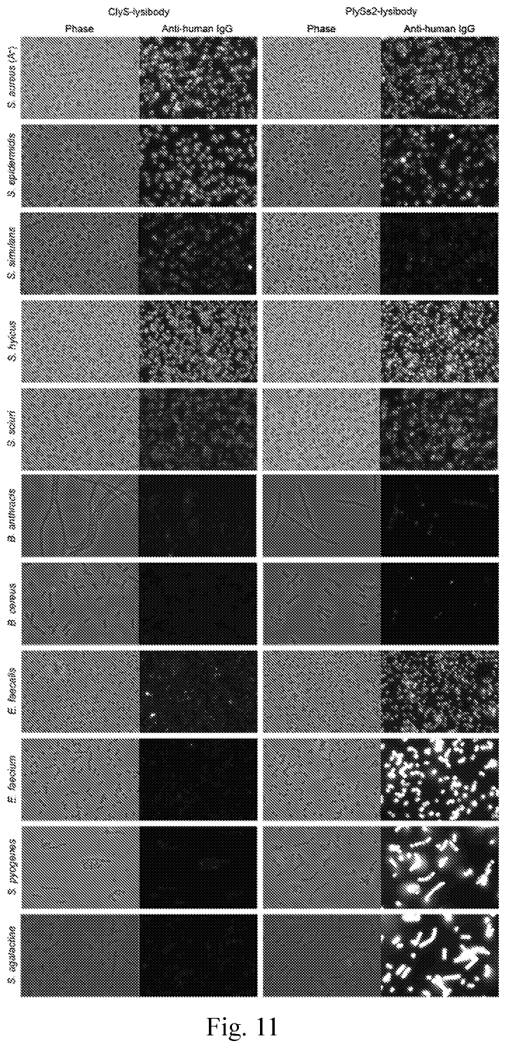

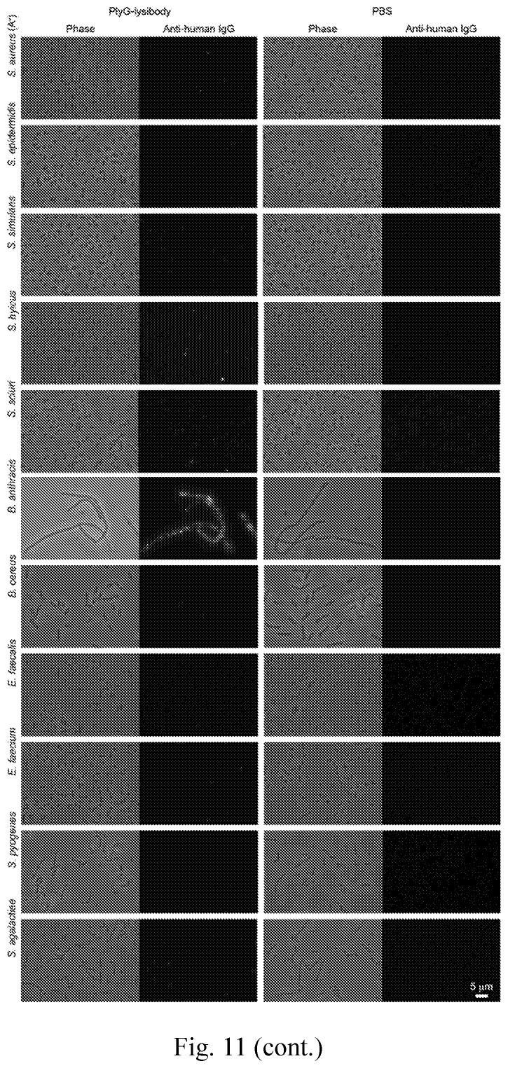

We further characterized the binding range of lysibodies to various staphylococcal species and other bacteria. AtlA-lysibody, ClyS-lysibody, and PlySs2-lysibody bound Staphylococcus epidermidis, Staphylococcus simulans, Staphylococcus hyicus, and Staphylococcus sciuri, in addition to S. aureus (FIGS. 10 and 11). For AtlA-lysibody and ClyS-lysibody, slight to no signal was observed for non-staphylococcal species (AtlA-lysibody bound weakly to Bacillus cereus and Enterococcus faecalis, exclusively at the septum). PlySs2-lysibody on the other hand, displayed a much broader host range, and bound in addition to staphylococci also E. faecalis, Enterococcus faecium, Streptococcus pyogenes, and Streptococcus agalactiae, consistent with the wider lytic activity range of the PlySs2 lysin. PlyG-lysibody specifically bound B. anthracis (FIG. 11).

### Example 3

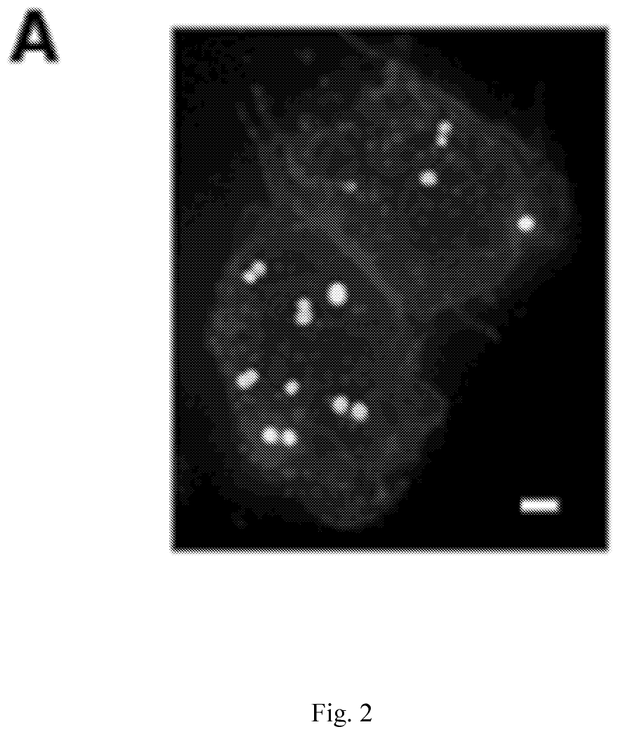

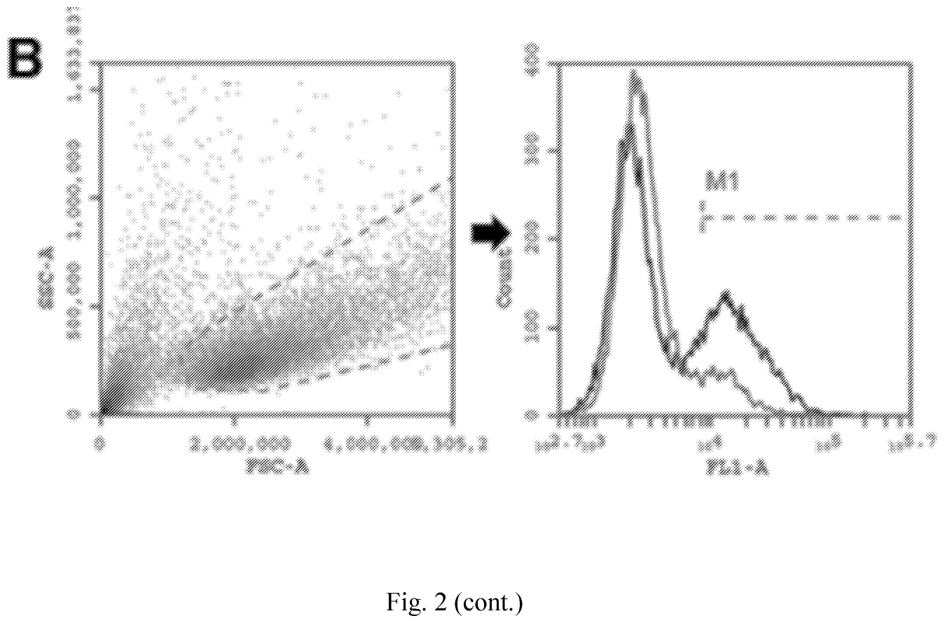

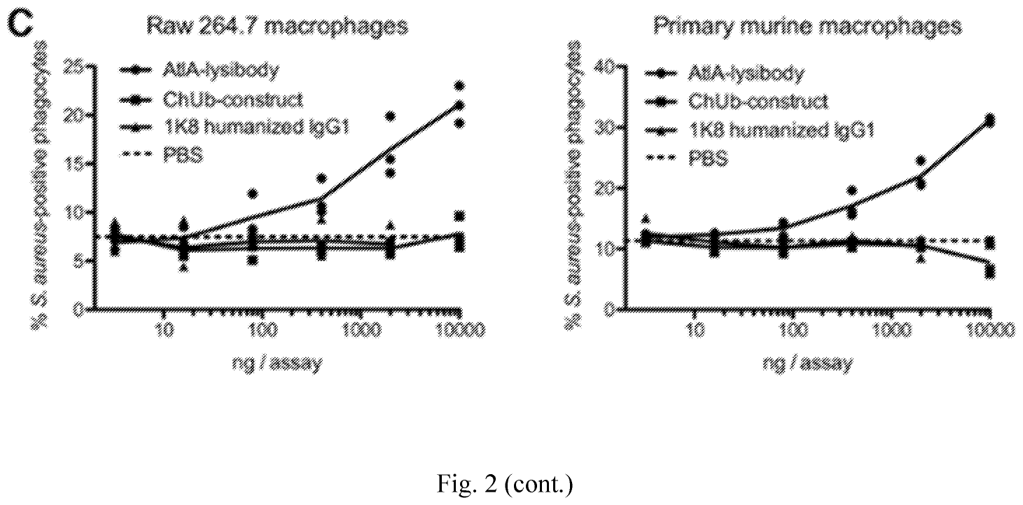

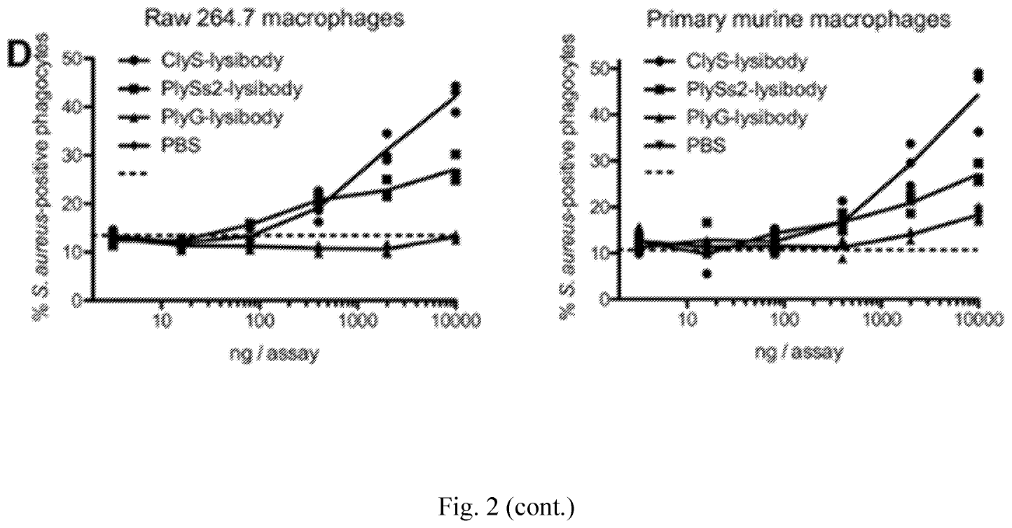

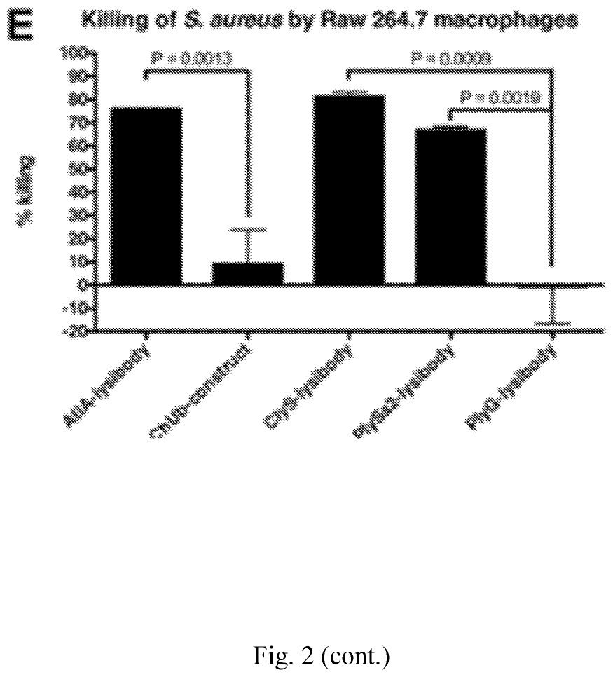

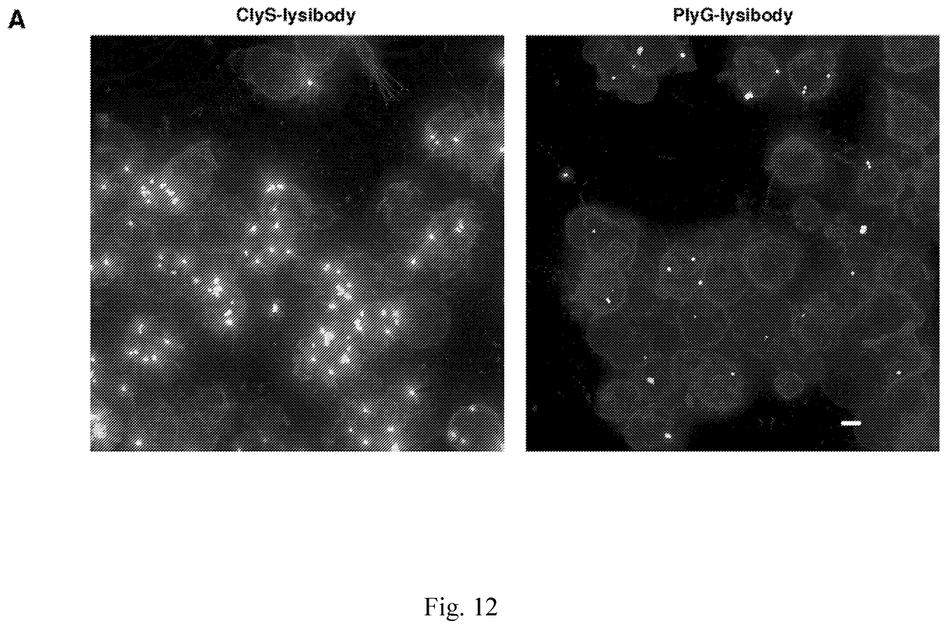

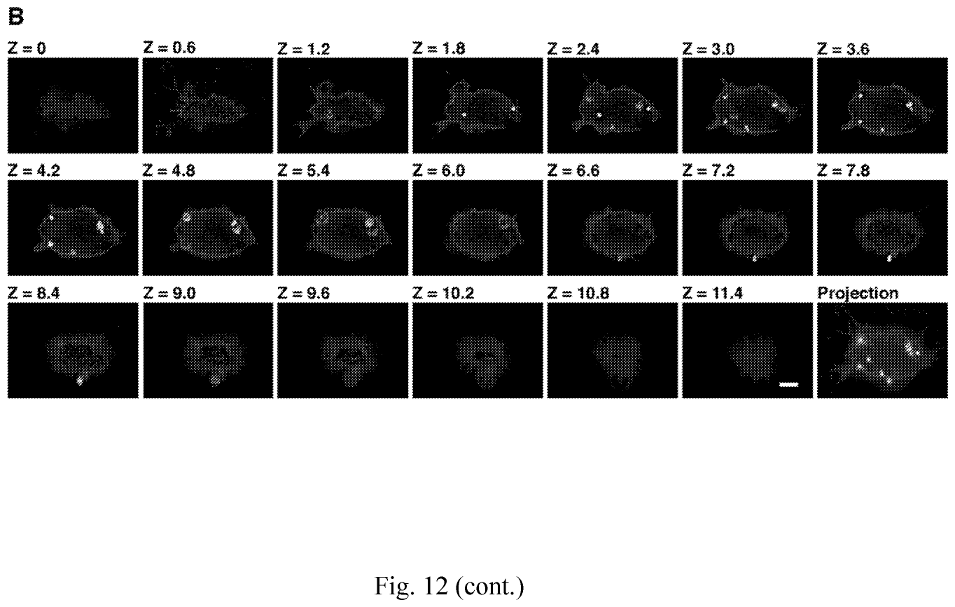

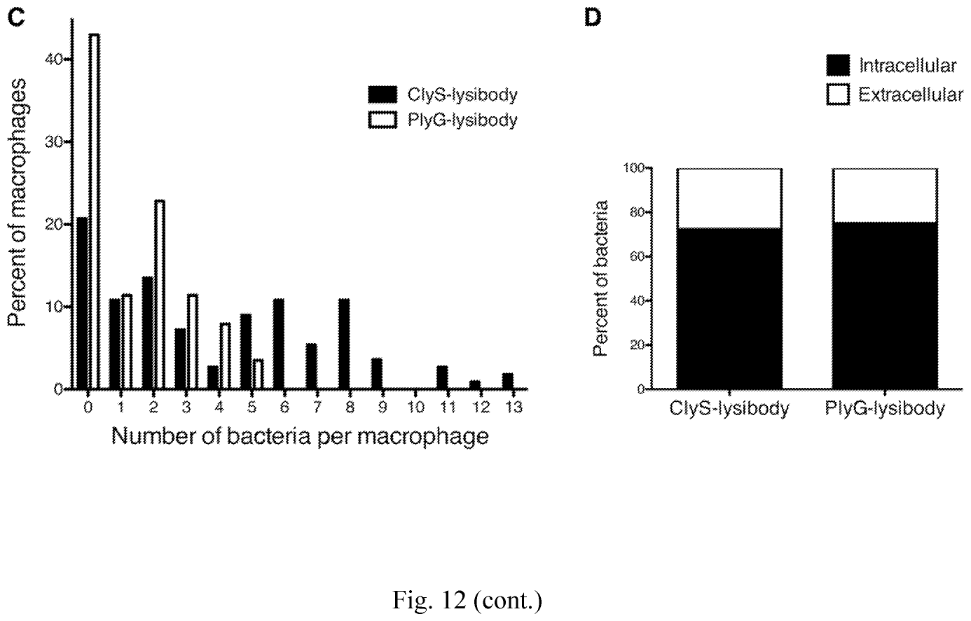

Lysibodies induce phagocytosis of S. aureus by macrophages Killing of S. aureus by phagocytes is an important immunological mechanism controlling this organism. To determine induction of phagocytosis, GFP-expressing staphylococci of strain Newman/pCN57 were incubated with a monolayer of macrophages for an hour in the presence of various lysibodies. Macrophages were washed and analyzed by microscopy (FIGS. 2A, and 12) and flow cytometry (FIG. 2B-D) to determine the extent of phagocytosis. We first gated on the macrophage population, and then determined the percentage of highly fluorescent macrophages, indicating a substantial staphylococcal load (FIG. 2B). AtlA-lysibody induced phagocytosis of staphylococci in a dose-dependent manner by both Raw 264.7 cell line macrophages and primary peritoneal murine macrophages, while the ChUb-construct and the non-specific monoclonal antibody 1K8 had no effect (FIG. 2C). Similarly, C-terminal fusion ClyS and PlySs2 lysibodies induced phagocytosis of S. aureus in a dose-dependent manner while the B. anthracis-specific PlyG-lysibody had no effect (FIG. 2D). To determine whether staphylococci are internalized or are merely attached to the surface of macrophages, we analyzed samples treated with ClyS-lysibody or PlyG-lysibody using deconvolution fluorescence microscopy, which allows 3D evaluation of the cells (FIG. 12). This analysis showed a much higher bacterial load in macrophages in the presence of ClyS-lysibody (FIG. 12A). Examination of sequential Z-sections allowed a clear distinction between intracellular and surface attached staphylococci (FIG. 12B). Analysis of a large population of cells showed that in the presence of ClyS-lysibody the staphylococcal load per macrophage increased dramatically compared to PlyG-lysibody (FIG. 12C). Roughly 75% of the staphylococci observed were inside macrophages, while 25% were associated with the surface or partially internalized (FIG. 12D).

### Example 4

Lysibodies Induce Fixation of Complement on the Surface of S. aureus

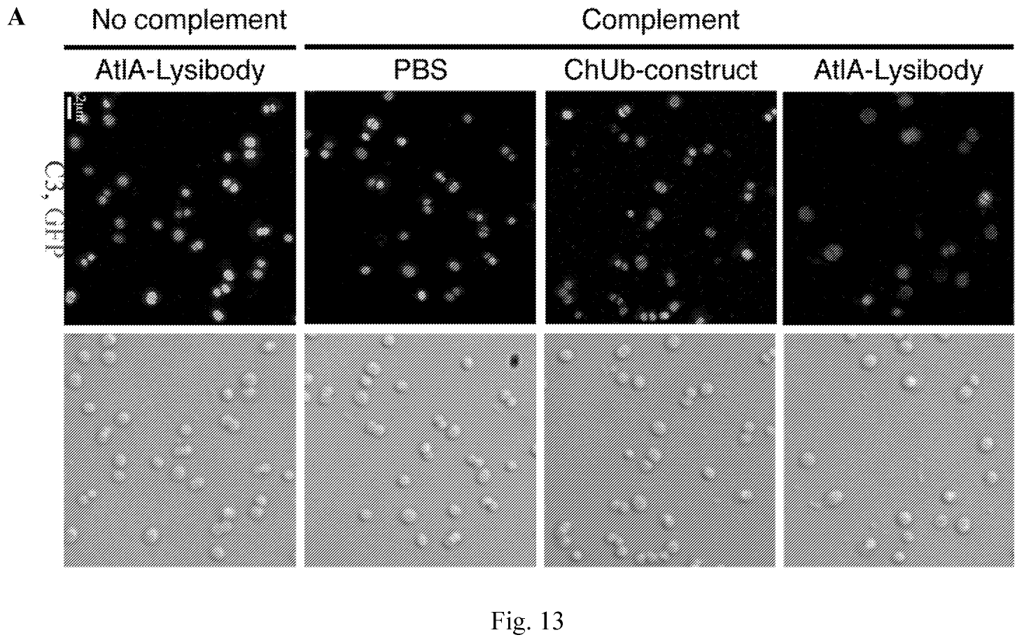

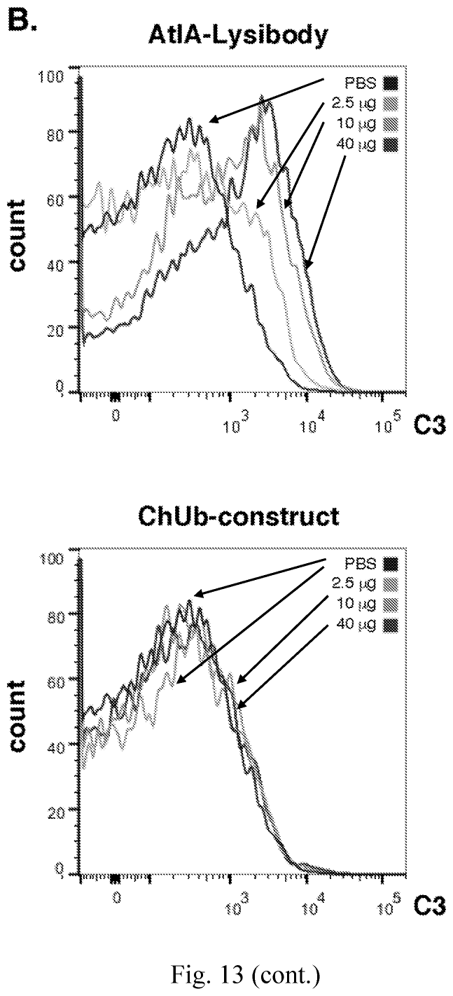

Complement deposition on the surface of pathogens is an important mechanism labeling them for removal by phagocytes. We used fluorescence microscopy to determine whether lysibodies can induce fixation of complement fragment C3b on the surface of S. aureus. Protein A negative strain Wood 46 was used to prevent non-specific fluorescent signal. Human serum that was pre-adsorbed on S. aureus to remove possible antibodies specific to this organism was used as a source of complement. AtlA, ClyS, and PlySs2 lysibodies induced complement deposition on the surface of the cells, although PlySs2-lysibody was less potent than the other two lysibodies (FIG. 3). Non-specific controls (ChUb-construct, 1K8 monoclonal antibody, PlyG-lysibody, and Fc alone) did not induce complement deposition. For AtlA-lysibody we also determined the extent of complement deposition on strain Newman/pCN57 using microscopy (FIG. 13A) and flow cytometry (FIG. 13B); protein A was blocked subsequent to complement deposition to prevent non-specific fluorescent signal. This analysis demonstrated that induction of complement deposition is dose-dependent.

### Example 5

Lysibodies Induce the Phagocytosis of S. aureus by Neutrophils

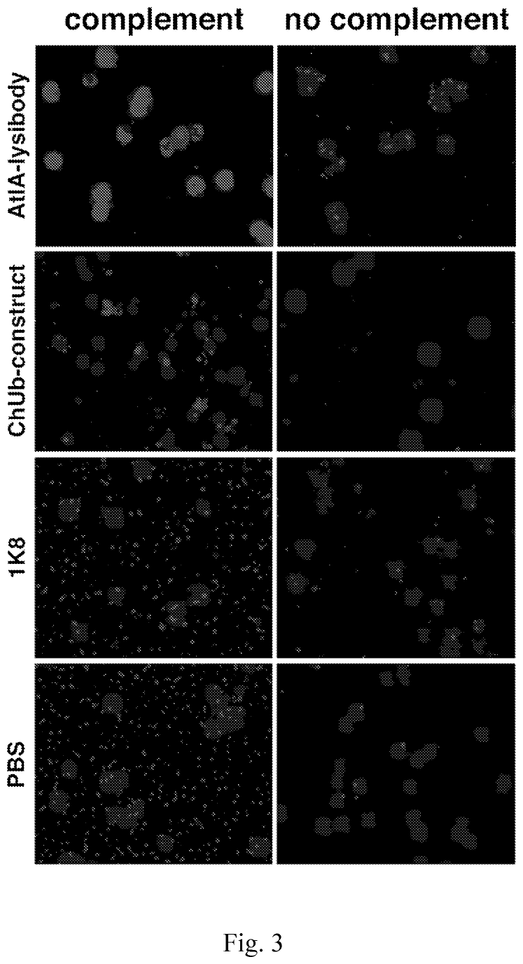

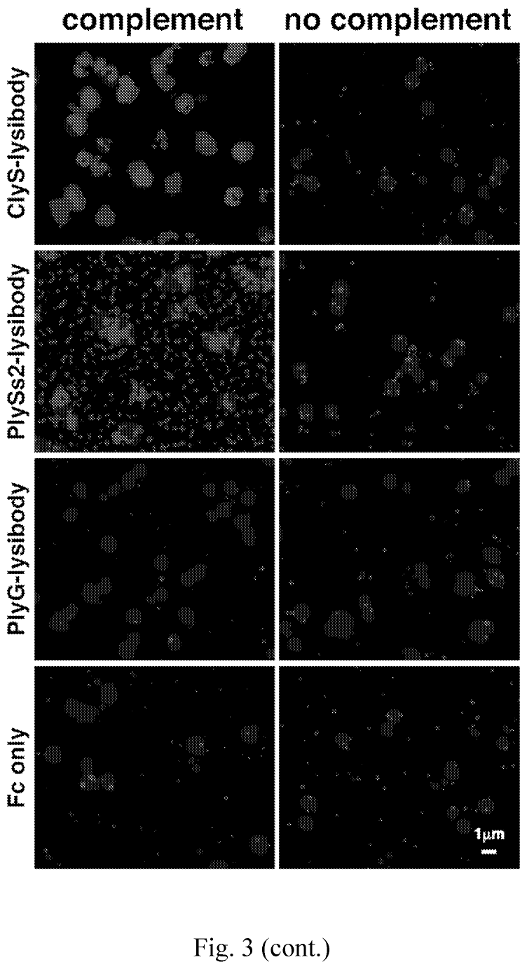

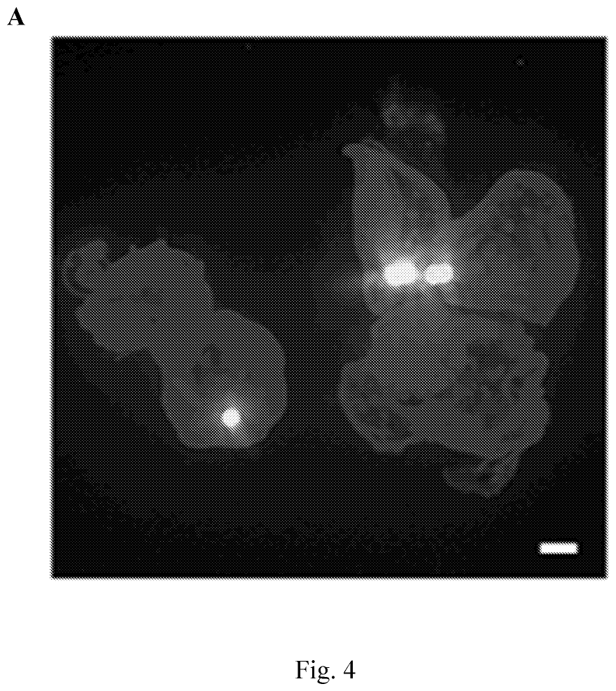

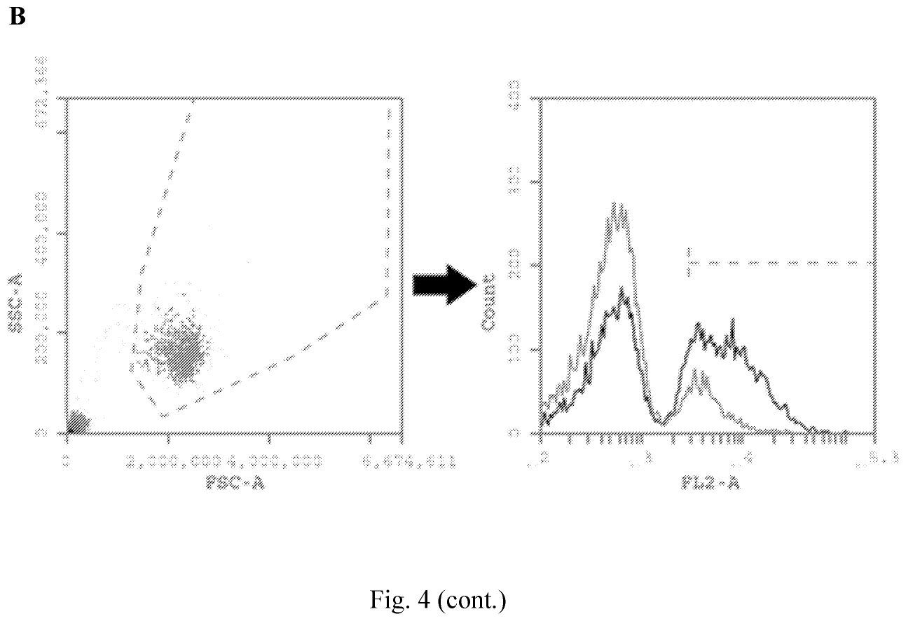

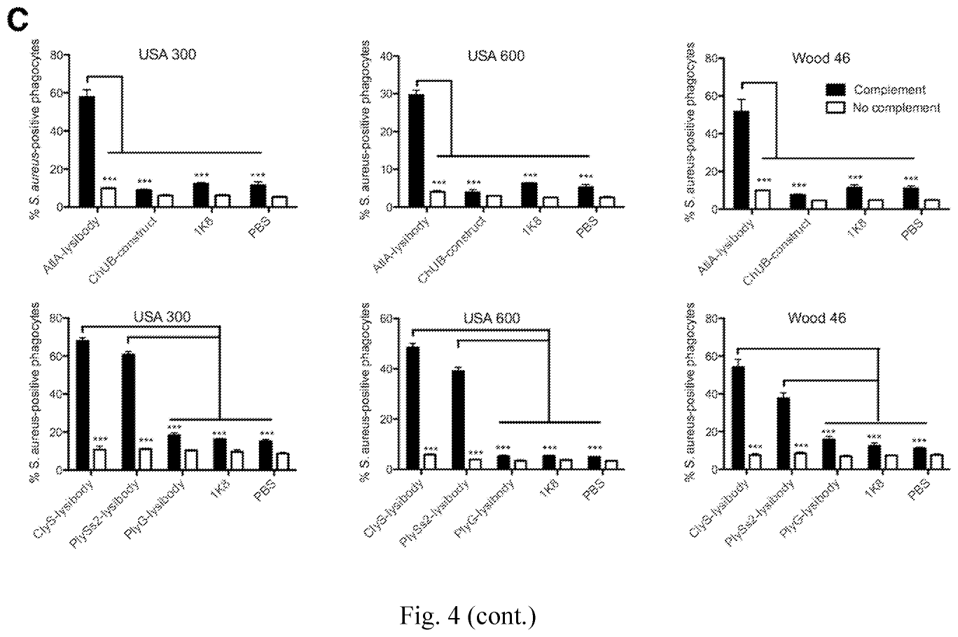

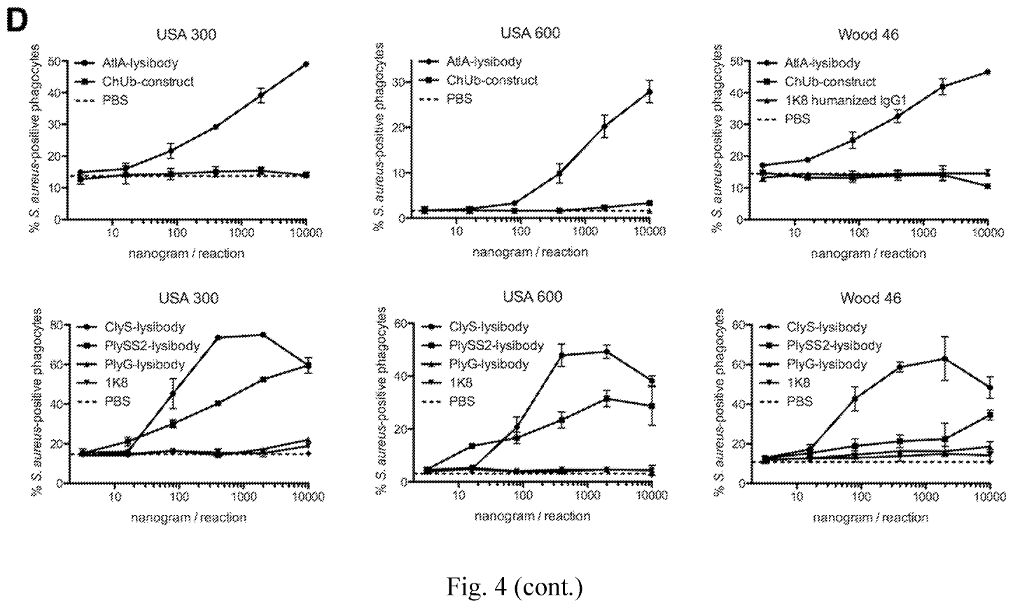

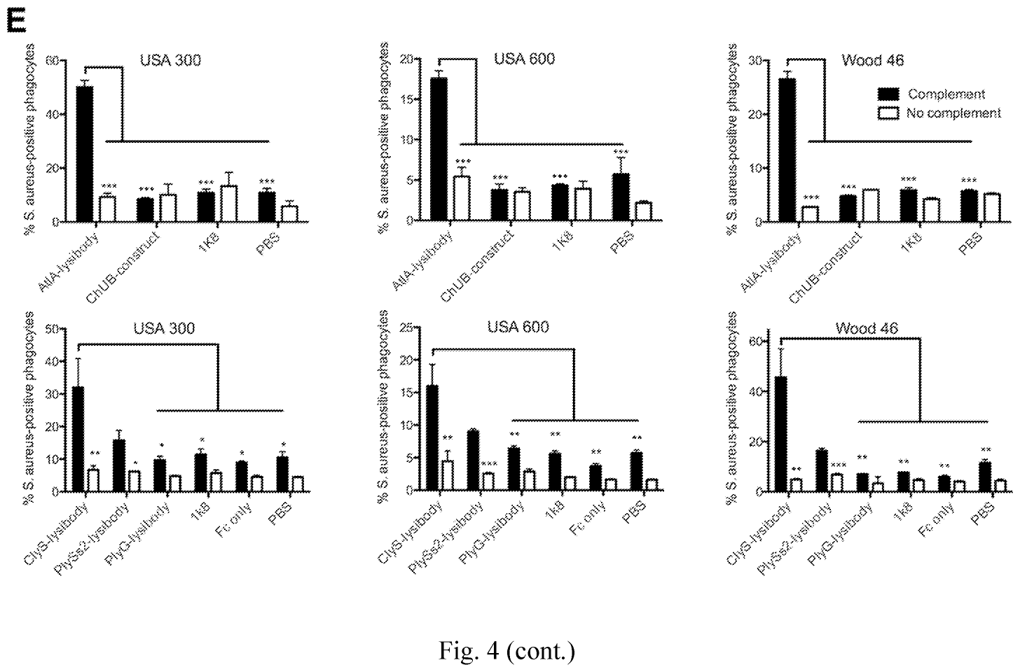

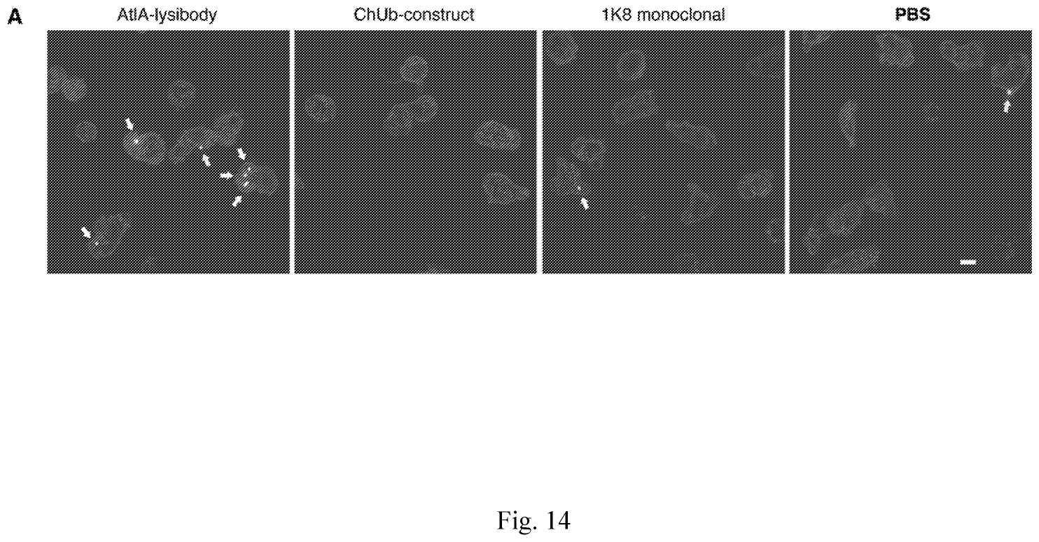

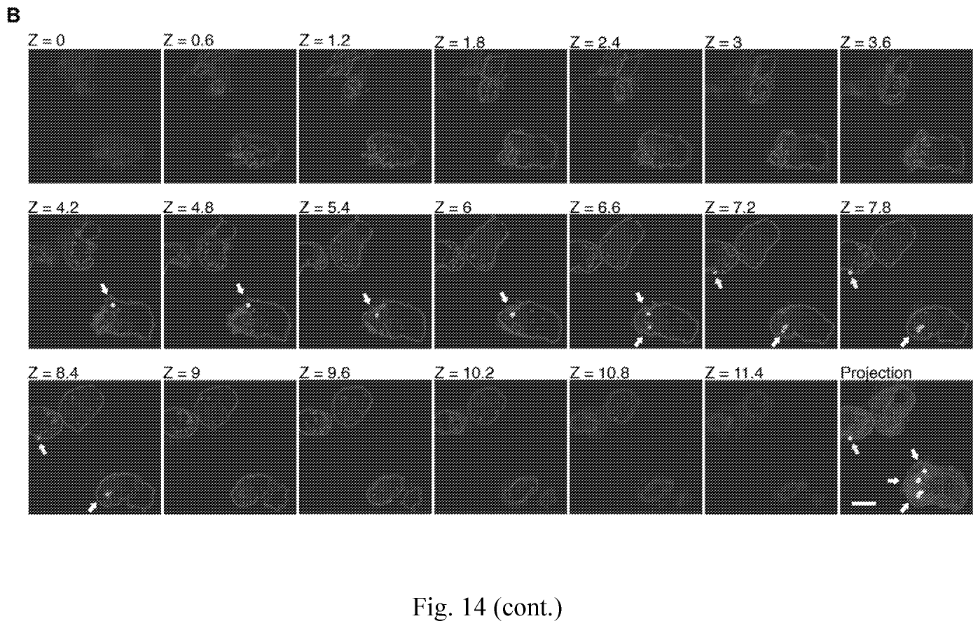

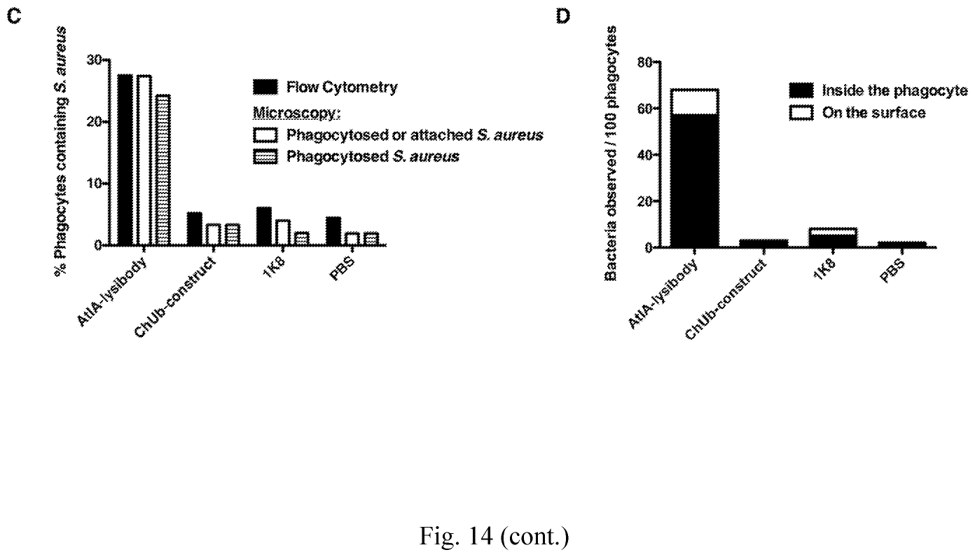

Neutrophils are the first line of defense against S. aureus infection. Direct recognition of Fc by FcγRs and recognition of surface-attached C3b by the C3-receptors, both play a role in promoting phagocytosis by neutrophils. We evaluated the phagocytosis of fluorescent S. aureus by neutrophils using fluorescence microscopy (FIGS. 4A and 14) and flow cytometry (FIG. 4B-E). We used FITC-labeled staphylococci for increased sensitivity. S. aureus-specific lysibodies (AtlA, ClyS, and PlySs2) induced phagocytosis of S. aureus by differentiated HL-60 neutrophils in a complement-dependent manner, while control constructs had no effect (FIG. 4C). Induction of phagocytosis was dose-dependent in all cases (FIG. 4D). AtlA-lysibody and ClyS-lysibody also induced phagocytosis of S. aureus in a complement-dependent manner using human polymorphonuclear cells, however PlySs2-lysibody was less effective with these cells (FIG. 4E).

To rule out the possibility that the flow cytometry results represent bacteria attached to the surface of neutrophils rather than phagocytosed bacteria, we analyzed samples treated with AtlA-lysibody or controls by both flow cytometry and deconvolution fluorescence microscopy, which allows 3D evaluation of the cells (FIG. 14). The percentage of neutrophils containing intracellular bacteria using this method closely resembled the percentage obtained using flow cytometry. Furthermore, only a minority of staphylococci where observed attached to the surface of neutrophils, and these were often associated with phagocytic cups.

### Example 6

Lysibodies Protect Mice from S. aureus Infection

Two infection models were used to test protection of mice from S. aureus infection. In a kidney abscess model, 1 mg ClyS-lysibody or control PlyG-lysibody were each injected into ten BALB/c female mice. Twenty-four hours later, the mice were challenged intraperitoneally with a sub lethal dose of 2.5×10⁶ CFU of the methicillin-resistant, vancomycin-intermediate S. aureus strain USA600. Five days later, surviving mice were sacrificed, and the bacterial load in the kidneys was determined through homogenization, serial dilution, and plating. Mice treated with ClyS-lysibody had a markedly reduced bacterial load compared to mice treated with control PlyG-lysibody (FIG. 5A).

To test protection from bacteremia, 0.3 mg AtlA-lysibody, control ChUb-construct, or PBS were each injected into female BALB/c mice. Twenty-four hours later, the mice were challenged intraperitoneally 2×10⁶ CFU of the methicillin-resistant S. aureus strain MW2 (USA400). Mouse viability was monitored for 8 days, at which time surviving mice were sacrificed. AtlA-lysibody had substantially improved survival rates compared to controls (FIG. 5B).

The following materials and methods were used to produce results in the foregoing Examples in this disclosure.

### Cell Lines, Bacteria, and Media

293T cells were obtained from Dr. Michel Nussenzweig at the Rockefeller University, and were grown in Dulbecco's Modified Eagle Medium (DMEM) containing 10% heat-inactivated fetal bovine serum (FBS, Sigma), and 2 mM sodium pyruvate (Sigma). Raw 264.7 murine macrophage cell line was obtained from ATCC (ATCC number: TIB-71), and grown in minimum essential media (MEM, Gibco, Life technologies), containing 1 mM sodium pyruvate, and 10% heat inactivated FBS. HL-60 cells were obtained from ATCC (ATCC number: CCL241), and propagated in RPMI 1640 (Gibco, Life Technologies), containing GlutaMax (Gibco, Life Technologies), 10% heat inactivated FB S, penicillin, and streptomycin. Differentiation of HL-60 was performed using a similar medium, lacking antibiotics, and supplemented with 100 mM N,N-dimethylformamide (DMF, ≥99.8% purity, Sigma), in accordance with established procedures. All mammalian tissue cultures were incubated at 37° C., 5% CO₂.

Bacterial strains used in this disclosure are denoted in Table 2. Staphylococcus aureus strain Newman/pCN57 was created by transforming strain Newman with plasmid pCN57, which expresses green fluorescent protein (GFP) from the strong PblaZ promoter. E. coli strains were grown in LB medium. Staphylococci, enterococci, and bacilli were grown in brain heart infusion (BHI) broth (BD). Streptococci were grown in Todd-Hewitt medium (Difco) supplemented with 1% yeast extract (Fisher Scientific). Bacterial strains were grown at 37° C. with shaking except for streptococci and enterococci, which were grown stationary at 37° C.

### Reagents

Dulbecco's phosphate buffered saline (DPBS) without calcium chloride and magnesium chloride was from Gibco, DPBS/Modified with calcium chloride and magnesium chloride was from HyClone. Goat serum was from Sigma. Goat anti-human IgG (gamma chain specific) alkaline phosphatase antibody (Sigma) was used at 1:5000 dilution for Western blots. Goat anti-Human IgG, Fcγ fragment specific, DyLight 594 conjugate (Jackson ImmunoResearch) was used at 1:1000 dilution. Polyclonal rabbit anti-human C3c Complement (Dako, A 0062, 9.6 g/L) was used at 1:500 dilution for microscopy and 1:2000 dilution for flow cytometry. Goat anti-rabbit IgG Alexa Fluor 594 conjugate highly cross-adsorbed (Life Technologies) was used at 1:1000 dilution for microscopy and 1:2000 for flow cytometry. Wheat germ agglutinin (WGA) Alexa Fluor 488, and Alexa Fluor 594 conjugates (Molecular Probes) was used at 5 μg/ml. DAPI (Sigma) was used at 1 μg/ml. Fluorescein isothiocyanate (FITC) isomer 1 (Sigma), and NHS-Rhodamine red (Thermo Scientific), were used according to manufacturer instructions. Other reagents were from Sigma unless otherwise noted.

### Construction of AtlA-Lysibody and ChUb-Construct Expression Vectors

The multi-cloning site of the mammalian expression vector AbVec-hIgG1, GenBank ID FJ475055, was replaced with a new multi-cloning site (AgeI-NotI-PstI-BamHI-XbaI-EcoRV-PvuII-SalI) by aligning primers abVec_MCS_5_AgeI_SalI and abVec_MCS_3-AgeI_SalI, and inserting the resulting double stranded DNA fragment, between the AgeI and SalI sites of AbVec-hlgG1, yielding pAR323.

pAR401_AtlA-lysibody was derived from pAR323 through the insertion of the following DNA fragments: A DNA fragment encoding amino acids TGHHHHHHGGGGSGGGSGR (SEQ ID NO:6), created by aligning primers H6GS_5_AgeI_NotI and H6GS_3_AgeI_NotI, was inserted between sites AgeI and NotI. A DNA fragment encoding the R1-R2 binding domains of S. aureus Newman AtlA was amplified using primers AtlA_R1R2_5_NotI and AtlA_R1R2_3_PstI, and the resulting PCR product was inserted into the NotI and PstI sites. The Fc region of human IgG1, encompassing the hinge region and constant domains 2 and 3, was amplified using primers hIgG1_Fc_5_PstI and hIgG1_Fc_3_HinDIII, and inserted between the PstI and HinDIII sites, thereby replacing the original human IgG1 Fc fragment found on this plasmid. Nucleotide changes encoded on primer hIgG1_Fc_5_PstI resulted in a change of the original N-terminal-most cysteine encoded on this fragment, which normally forms a disulfide bridge with the light chain, to a glycine.

To construct the control plasmid pAR444_ChUb-construct, a single chain Fv fragment specific for chicken ubiquitin that was obtained from the domain antibody phage library was amplified using primers ChickUbi_5 NotI and ChickUbi_3_PstI, and inserted into the NotI and PstI sites of pAR401_AtlA-lysibody, thereby replacing the AtlA binding repeats R1-R2. The Fc-alone construct was created by amplifying the human IgG1 Fc region using primers hIgG1_Fc-only_5_NotI and hIgG1_Fc_3_HinDIII, and inserting the resulting PCR product between the NotI and HinDIII sites of pAR401_AtlA-lysibody, yielding pAR450_Fc-only.

For the creation of C-terminal fusion lysibodies, the multi-cloning site of pAR323 was further modified by replacing the region between the PstI and SalI restriction sites with a DNA fragment obtained by the alignment of primers New_MCS_5_PstI_SalI and New_MCS_3_PstI_SalI, which contains the following restriction sites: PstI-BamHI-XbaI-BglII-EcoRV-SalI. A DNA fragment encoding a hexahistidine tag and a short glycine/serine linker was created by aligning primers H6GS_5_AgeI_NotI and H6GS_3_AgeI_NotI, was inserted between the AgeI and NotI restriction sites. The hinge region and constant domains CH2 and CH3 of human IgG1 were amplified using primers hIgG1_Fc_5_NotI and hIgG1_Fc_3_PstI, and inserted into the NotI and PstI sites; a cysteine in the hinge region, which normally forms the disulfide bond with the light chain, was changed to a glycine.

Various phage lysin binding domains were cloned into the SalI and HinDIII sites of the resulting plasmid as follows: The ClyS binding domain was amplified with primers ClyS-BD_5_SalI and ClyS-BD_3_HinDIII, yielding pAR422_ClyS-lysibody. The PlySs2 binding domain was amplified with primers PlySs2-BD_5_SalI and PlySs2-BD_3 HinDIII, yielding pAR423PlySs2-lysibody. The binding domain of PlyG was amplified with primers PlyG-BD_5_SalI and PlyG-BD_3 HinDIII, yielding pAR465PlyG-lysibody.

### Construction of Lysin Binding Domains GFP Fusion Proteins

A fusion of the binding domain from the ClyS lysin to GFP was produced by inserting the following PCR products into pBAD24: The ClyS binding domain was amplified using primers ClyS-BD_5_XbaI and ClyS-BD_3 PstI and inserted between the XbaI and PstI sites. GFP_mut2 was amplified using primers H6_GFP_5_EcoRI GFP_3_KpnI (a hexahistidine tag was added on the 5′ primer) and inserted between the EcoRI and KpnI sites, yielding pAR160_GFP_ClyS-BD. The binding domain of PlySs2 was amplified using primers PlySs2-BD_5_XbaI and PlySs2-BD_3 PstI, and the resulting PCR product was inserted into the XbaI and PstI sites of pAR159 (a pBAD24-based plasmid, containing a hexahistidine-tagged GFP), yielding pAR517_GFP_PlySs2-BD.

A control construct containing only a hexahistidine-tagged GFP was produced by amplifying the GFP_mut2 gene using primers H6_GFP_5_EcoRI and GFP_3_PstI, and inserting the resulting PCR product into the EcoRI and PstI sites of pBAD24, yielding pAR518_GFP.

### Expression and Purification of Lysibodies

Lysibodies and similar constructs were produced in 293T cells using polyethylenimine (PEI) transient transfection method. For a 1 L reaction 2 mg of the expression vector DNA, and 500 μg helper vector were mixed in 10 ml optimem medium (Gibco, Life Technologies). 10 ml optimem medium containing 2.5 mg PEI was then mixed in, and the reaction was left at the room temperature for 15 min. The mix was then added to 1 L DMEM medium containing 2 mM pyruvate, and 10 ml Nutridoma-SP (Roche). Alternatively, 1 L FreeStyle 293 Expression Medium (Life Technologies) was used. 293T cells were grown in 15 cm tissue culture plates to 70-80% confluence, washed with DPBS/modified containing calcium and magnesium (HyClone), and incubated with 20 ml transfection mix per plate for 6 days.

For N-terminal fusion lysibodies and monoclonal antibodies that do not contain a hexahistidine tag, medium from transfection plates was spun down, filtered, and proteins were precipitated with 60% ammonium sulfate at 4° C. overnight. Samples were centrifuged at 6000 RPM using a Sorvall RC-5B centrifuge equipped with a GS-3 rotor. The protein pellet was suspended in 25 ml PBS containing two tablets of Complete protease inhibitor cocktail tablet (Roche), and dialyzed against PBS using a membrane with 12-14 kDa cutoff (Spectrum Laboratories) for 24 h at 4° C. with 3 buffer changes. The dialyzed mix was centrifuged to remove precipitates and mixed end-over-end with protein G Sepharose beads (GE Healthcare) at 4° C. overnight. The protein G beads were loaded on a column and washed with 20 column volumes of PBS. The construct was eluted with 3 ml 0.1 M glycine pH 2.7 three times and each eluted fraction was immediately neutralized with 450 μl 1M tris pH 9.0. Positive fractions were concentrated using an Amicon ultrafiltration device with a 10 kDa molecular weight cutoff membrane. The buffer was changed to DPBS/modified trough three cycles of volume reduction and dilution in DPBS/modified. Protein concentration was determined according to absorbance at 280 nm, using a ND-1000 spectrophotometer (Nanodrop). The final product was stored in aliquots at −80° C.

Supernatants of C-terminal fusion lysibodies was filtered through a 0.22 m filter (Millipore) and loaded on a NiNTA column, calibrated with MCAC buffer (30 mM Tris pH 7.4, 0.5 M NaCl, 10% glycerol). The column was washed thoroughly with MCAC buffer and MCAC containing 20 mM imidazole. Lysibodies were eluted with MCAC containing 150 mM imidazole, and positive fractions were processed as described above. Purification of GFP binding domain fusion protein was done using metal affinity chromatography as previously described.

### Fluorescence Microscopy—Binding of Constructs to the Bacterial Surface

Bacteria were fixed for 15 min at room temperature, and 30 min on ice, using 2.6% paraformaldehyde, 0.012% glutaraldehyde, and 30 mM phosphate buffer pH 7.4. Fixed cells were washed with PBS, and attached to poly-L-lysine coated cover glass. The cells were washed and blocked for 15 min with 10% normal goat serum. For bacteria not expressing protein A, lysibody was diluted to 2 μg/ml in PBS containing 2% BSA and 1% gelatin and fluorescent conjugates were diluted 1:1000. Bacteria were incubated with each for 1 h at room temperature. Microscopy using binding domain—GFP fusions was done in a similar manner, using PBS containing 2% BSA and 1% gelatin as blocking agent and dilution buffer. Microscopy studies using lysibodies and clinical S. aureus strains that express protein A were performed by blocking fixed cells with PBS 2% BSA 1% gelatin, 10% goat serum, and 20% human serum sequentially. Lysibodies were conjugated to Rhodamine red according to manufacturer instruction (Thermo scientific), diluted to 5 μg/ml in PBS containing 2% BSA and 1% gelatin, and incubated with the cells for 1 h at room temperature. Slides were mounted in 50% glycerol and 0.1% p-phenylenediamine in PBS pH 8. Phase microscopy was performed using a Nikon Eclipse E400 microscope, equipped with a Nikon 100×/1.25 oil immersion lens, and a Retiga EXi fast 1394 camera (QImaging). QCapture Pro version 5.1.1.14 software (QImaging) was used for image capture and processing. Deconvolution microscopy was performed using a DeltaVision image restoration microscope (Applied Precision/Olympus) equipped with CoolSnap QE cooled CCD camera (Photometrics). An Olympus 100×/1.40 NA, UPLS Apo oil immersion objective was used in conjunction with a 1.5× optovar. Z-stacks were taken at 0.15 m intervals. Images were deconvolved using the SoftWoRx software (Applied Precision/DeltaVision), and corrected for chromatic aberrations.

### Raw 264.7 Phagocytosis Assay

S. aureus Newman/pCN57 were streaked on a BHI plate containing 10 μg/ml erythromycin, and were grown at 37° C. for one day, and then at 25° C. for an additional day. S. aureus cells from several separate colonies were scraped off the plate, washed once in PBS, and resuspended to a final OD₆₀₀ 0.3. Raw 264.7 macrophages were seeded at 5×10⁵ cells per well of a 24-well plate two days prior to the experiment. The wells were washed with 1 ml PBS, and supplemented with 300 μl MEM medium without serum, constructs at different concentrations, and 30 μl bacteria (roughly 1×10⁷ cells); the plates were incubated at 37° C. 5% CO₂ for 1 h. The wells were washed three times with PBS to remove extracellular bacteria, and the cells were fixed using 1 ml/well 1% paraformaldehyde in PBS for 1 h at 4° C. Each well was then washed with 1 ml PBS and the cells were scraped off the plate in 200 μl PBS, using a 10 μl disposable inoculation loop. The cell suspension was transferred to a U-bottomed 96-well plate and analyzed using a C6 flow cytometer (BD-accuri), with the CFlow software. Forward and side scatter were used to gate the macrophage population. Macrophages displaying elevated fluorescence in the green channel were denoted as positive for phagocytosis of S. aureus. Similar samples were processed in glass-bottomed wells, stained with WGA Alexa Fluor 594, and analyzed by deconvolution microscopy to verify the presence of fluorescent S. aureus within macrophages.

### Phagocytosis Assay—Murine Peritoneal Macrophage

5-6 weeks old female BALB/c mice were injected intraperitoneally with 1 ml of Brewer thioglycollate medium modified (BD). Mice were sacrificed after 4-5 days and the peritoneal cavity was washed with DPBS without calcium and magnesium to obtain macrophages. Macrophages were washed, and 5×10⁵ cells were added to each well of a 24-well plate, and incubated for 1 h at 37° C. 5% CO₂. The wells were washed 3 times with DPBS to remove non-adherent cells, and supplemented with 1 ml MEM medium without serum. Phagocytosis assays were performed as described for the Raw 264.7 cells, however, detachment of adherent cells following fixation was done using 250 μl 0.25% trypsin in PBS pH 7.2, 0.1% EDTA, for 30 min at 37° C., followed by gentle pipetting with a 1 ml pipette tip.

Preparation of Human Complement, Adsorbed on S. aureus

Blood was obtained from a healthy human donor by venipuncture, and was immediately placed on ice for 2 hours until clotting occurred. Clots were removed by centrifugation and the serum passed through a 0.22 μm filter. EDTA was added to a final concentration of 25 mM to prevent complement activation, and the serum was adsorbed on S. aureus Newman/pCN57. For each ml of serum adsorbed, a washed pellet from 10 ml overnight culture and 10 ml late logarithmic stage culture were used, to account for possible variability of surface epitopes between growth stages. The serum was rotated end-over-end with S. aureus cells for 30 min at 4° C., centrifuged to remove cells, and filtered through a 0.22 m filter. The serum was then dialyzed against 1.5 L 5 mM HEPES 0.9% NaCl pH 7.4, using a membrane with cutoff limit of 12-14 kDa, for 16 h with two buffer changes. The serum was then frozen in liquid nitrogen as single-use aliquots, and stored at −80° C. until use.

### Complement Fixation—Sample Preparation for Microscopy

S. aureus Wood 46 (protein A negative) was grown on a BHI plate for a day at 37° C., and then a day at 25° C. Several separate colonies were scraped off the plates and suspended in PBS to a final OD₆₀₀ 1.0. The cells were attached to an acid-washed poly-L-lysine coated cover slides. Lysibodies and other constructs were diluted to a final concentration of 1 mg/ml in DPBS containing calcium and magnesium (Hyclone), and 10 μl were added to each slide and incubated at room temperature for 1 h. The cells were washed 3 times with PBS and once with DGHB (5 mM HEPES, 71 mM NaCl, 0.15 mM CaCl₂, 0.5 mM MgCl₂, 2.5% glucose, 0.1% gelatin, pH 7.4), and incubated for 20 minutes at 37° C. with 30 μl DGHB containing 0.5% human serum that was adsorbed on S. aureus (see above). The cells were washed thoroughly with PBS and fixed with 50 μl 2.6% paraformaldehyde in PBS for 1 h at 4° C. The cells were then washed again with PBS and blocked with PBS 2% BSA 1% gelatin overnight at 4° C. The slides were washed with PBS and incubated with 10 μl rabbit anti-C3 diluted 1:500, followed by 3 PBS washes, and then incubated with 10 μl goat anti-rabbit Alexa Fluor 594 conjugate diluted 1:1000, and 1 μg/ml DAPI; incubation steps were 1 h at room temperature each. The slides were mounted and imaged as described above. For microscopy on Newman/pCN57 (Protein A positive, expressing cytoplasmic GFP), the cells were blocked following fixation using PBS 2% BSA 1% gelatin, followed by heat inactivated goat and human sera.

### Complement Fixation—Sample Preparation for Flow Cytometry

S. aureus Newman/pCN57 were grown on a BHI plate for one day at 37° C. and for another day at 25° C. Several separate colonies were scraped off the plates and suspended in PBS to a final OD₆₀₀ 1.0. 30 μl bacteria were mixed with lysibodies or controls at various concentrations, and the final volume was adjusted to 200 μl with PBS. The cells were rotated at 4° C. for two hours, and washed with 0.5 ml saline and then 100 μl GVB (gelatin veronal buffer, Sigma). The cells were suspended in 300 μl 3% human complement (adsorbed on S. aureus, see above) in GVB, and the tubes were rotated at 37° C. for 15 min. The samples were then immediately placed on ice, and EDTA was added to a final concentration of 20 mM to stop complement fixation. The samples were washed twice with PBS, and fixed with 250 μl 2.6% paraformaldehyde in PBS (phosphate adjusted to 40 mM, pH 7.4) for 1 h at 4° C. The samples were then washed twice with PBS, and the pellet was blocked with 100 μl PBS 2% BSA 1% gelatin for 20 min. 100 μl 10% heat-inactivated goat serum were added for an additional 20 minutes, and then 10 μl heat-inactivated human serum were added to each sample for an additional 20 min, in order to block protein A. The cells were then washed with PBS, and each tube was suspended in 100 μl rabbit anti C3 antibody diluted 1:2000 in PBS 2% BSA 1% gelatin, and rotated for 1 h at room temperature. The cells were washed with PBS, suspended in 100 μl goat anti rabbit Alexa Fluor 594 conjugate diluted 1:2000 in PBS 2% BSA 1% gelatin, and the tubes were rotated for 1 h at room temperature. The cells were then washed with PBS and resuspended in 200 μl PBS. Samples were analyzed using a BD-Accuri C6 flow cytometer, and the CFlow and FlowJo softwares. Unlabeled and mono-labeled samples were used to calibrate compensation values. Gating was done on GFP-positive cells to exclude non-S. auereus particles, and C3b signal distribution was determined.

Phagocytosis of S. aureus by HL-60 Neutrophils, and Human Peripheral PMNs

HL-60 neutrophils were propagated and differentiated according to known approaches. Primary human neutrophils were isolated from blood of healthy volunteers collected in tubes containing acid citrate dextrose (ACD). For each 9 ml of blood, 4.5 ml of 6% dextran (Mw ˜100,000, Sigma) 0.9% NaCl was added. The tubes were left stationary at room temperature for 30 minutes to allow red blood cells to settle, and the top layer was collected. The cells were centrifuged, and residual red blood cells were lysed for 30 sec in 0.2% NaCl, and the solution was supplemented by an equal volume of 1.6% NaCl. The cells were centrifuged and the process was repeated two more times. The cells were then suspended in 10 ml DPBS (without calcium and magnesium), and 3 ml Ficoll-Hypaque solution (density 1.077 g/L) was layered at the bottom of the tube. The cells were centrifuged at 900 g for 20 min at 20° C., and the cell pellet was washed once with DPBS without calcium and magnesium, and suspended in HBSS 0.1% gelatin at a final concentration of 1×10⁸ cells/ml.

For phagocytosis assays, S. aureus strains were grown on BHI plates for one day at 37° C. and for another day at 25° C. Several separate colonies were scraped off the plates and resuspended in PBS to a final OD₆₀₀ 1.0, and fixed for 1 h at 4° C. with 2.6% paraformaldehyde in PBS (phosphate concentration adjusted to 40 mM, pH 7.4). Bacterial cells were labeled with fluorescein isothiocyanate (FITC, Sigma) according to manufacturer instructions, and washed thoroughly. Cells were suspended in PBS 14% glycerol, and frozen in single use aliquots at −80° C. Upon thawing, S. aureus cells were adjusted to 5×10⁷ cells/ml, in Hanks' balanced salt solution (HBSS, Gibco 14025) containing 0.1% gelatin. Phagocytosis assay was a modification of a known approach. To each well of a round-bottomed 96-well plate were added 60 μl HBSS 0.1% gelatin, 10 μl lysibody or control diluted in HBSS 0.1% gelatin, and 10 μl diluted bacteria. The plate was shaken at 200 RPM, 4° C. for 1 h. Then, 10 μl of 5% S. aureus-adsorbed complement (see above), and 10 μl HL-60 cells or primary human neutrophils adjusted to 1×10⁸ cells/ml in HBSS 0.1% gelatin were added to each well, and the plate was shaken at 200 RPM at 37° C. for 1 h. Cells were then fixed in 2.6% paraformaldehyde, 30 mM sodium phosphate pH 7.4, for 1 h at 4° C. The cells were then washed, suspended in 200 μl PBS and analyzed using a C6 flow cytometer (BD-accuri). Forward and side scatter were used to gate the neutrophil population, and cells displaying an increase in fluorescence were denoted as positive for phagocytosis of S. aureus. A sample of the cells was stained with WGA Alexa Fluor 594 and analyzed by deconvolution microscopy to verify the presence of fluorescent S. aureus within neutrophils.

### Mouse Kidney Abscess Model

The mouse model was modified from an existing model. Five weeks old BALB/c female mice (Jackson Laboratories, Bar Harbor, Me.) were each injected intraperitoneally with 1 mg lysibody or control, in a total volume of 500 μl. S. aureus strain USA600 (MRSA, VISA) was diluted 1:100 in BHI from an overnight culture and grown at 37° C. with shaking at 200 RPM to OD₆₀₀ 0.5. Cells were harvested, washed with saline, and suspended in saline to OD₆₀₀ 1.0. Bacteria were diluted in saline, and adjusted to 2.5×10⁶ CFU per mouse (injection volume 500 μl) in saline 5% hog gastric mucin (Sigma). Injection of mice was performed 24 h following injection of lysibodies. Actual injected CFU was determined through plating. Mouse viability was monitored every 24 h, and after 4 days surviving mice were sacrificed. Both kidneys were dissected and ground in 1 ml 0.5% saponin. Samples were serially diluted and streaked on BHI plates for quantification of bacterial load. Data analysis was done using Prism version 5.0c (GraphPad Software, La Jolla, Calif.). Primers used in this disclosure are presented in Table 1. The sequences in the accompanying sequence listing are all given in the 5′->3′ direction.

TABLE 1
Primers

| Primer name | Sequence | SEQ ID No. |
|---|---|---|
| abVec_MCS_5_AgeI_SalI | CCGGTAGCGGCCGCCTGCAGGGATCCTCTAGAGATATCC | 7 |
| | AGCTGA AG | |
| abVec_MCS_3_AgeI_SalI | TCGACTTCAGCTGGATATCTCTAGAGGATCCCTGCAGGC | 8 |
| | GGCCGCT A | |
| H6GS_5_AgeI_NotI | CCGGTCATCATCATCATCATCATGGAGGAGGAGGAAGCG | 9 |
| | GAGGAG GAAGC | |
| H6GS_3_AgeI_NotI | GGCCGCTTCCTCCTCCGCTTCCTCCTCCTCCATGATGAT | 10 |
| | GATGATG ATGA | |
| At1A_R1R2_5_NotI | CCCGCGGCCGCATGACAACTACCCCTACTACACCATCAA | 11 |
| | AACC | |
| At1A_R1R2_3_PstI | GGGCTGCAGAGCTGTTTTTGGTTGTGCTACTGC | 12 |
| hIgG1_Fc_5_PstI | CCCCTGCAGCCCAAATCTGGTGACAAAACTC | 13 |
| hIgG1_Fc_3_HinDIII | CTTAAGCTTTCATTTACCCGGAGACAGGG | 14 |
| hIgG1_Fc_5_NotI | CAAGCGGCCGCCCCAAATCTGGTGACAAAACTC | 15 |
| hIgG1_Fc_3_PstI | CACCTGCAGTTTACCCGGAGACAGGGAG | 16 |
| New_MCS_5_PstI_SalI | GGGGGGATCCGGGTCTAGAGGAAGATCTGGAGGAGGAGG | 17 |
| | GGATA TCAAG | |
| New_MCS_3_PstI_SalI | TCGACTTGATATCCCCTCCTCCTCCAGATCTTCCTCTAG | 18 |
| | ACCCGGA TCCCCCCTGCA | |
| ClyS-BD_5_SalI | CCGGTCGACCATGAATAAGATCACAAATAAAGTTAAACC | 19 |
| | ACC | |
| ClyS-BD_3_HinDIII | CCCAAGCTTTTAAAACACTTCTTTCACAATCAATCTCTC | 20 |
| P1ySs2-BD_5_SalI | GAGGTCGACCACACCGCCTGGCACGGTCGCACAG | 21 |
| P1ySs2-BD_3_HinDIII | GAGAAGCTTTTATTTAAATGTACCCCAAGCATTG | 22 |
| ChickUbi_5_NotI | GGGGCGGCCGCATGGCCGAGGTGCAGCTGTTGGAG | 23 |
| ChickUbi_3_PstI | CCCCTGCAGTCGTTTGATTTCCACCTTGGTCCCTTG | 24 |
| PlyG-BD_5_Sa1I | GGGGTCGACTCATGTGGCGACTACTTCACC | 25 |
| PlyG-BD_3_HinDIII | CCCAAGCTTTTATTTAACTTCATACCACCAACC | 26 |
| hIgG1_Fc-only_5_NotI | CCCGCGGCCGCCCCAAATCTGGTGACAAAACTC | 27 |
| ClyS-BD_5_XbaI | CCGTCTAGAATGAATAAGATCACAAATAAAGTTAAACCAC | 28 |
| | C | |
| ClyS-BD_3_PstI | GCGCTGCAGTTAAAACACTTCTTTCACAATCAATCTCTC | 29 |
| H6_GFP_5_EcoRI | CGCGAATTCATGAGTAAAGGAGAACTTCATCATCATCAT | 30 |
| | CATCATTCCTCCGCCATGAGTAAAGGAGAAGAACTTTTC | |
| GFP_3_KpnI | GAGGGTACCTTTGTATAGTTCATCCATGCC | 31 |
| PlySs2-BD_5_XbaI | GAGTCTAGAACACCGCCTGGCACGGTCGCACAG | 32 |
| PlySs2-BD_3_PstI | GGGCTGCAGTTATTTAAATGTACCCCAAGCATTG | 33 |
| GFP_3_PstI | CGCCTGCAGTTATTTGTATAGTTCATCCATGCCATGTG | 34 |

Restriction sites are underlined.

TABLE 2
Bacterial strains used in this disclosure

| Organism | Source |
|---|---|
| Bacillus anthracis, ΔSterne | Daniel, A. et al . . . Antimicrob |
| | Agents Chemother 54, 1603- |
| | 1612 (2010). |
| Bacillus cereus, T | Daniel, A. et al . . . Antimicrob |
| | Agents Chemother 54, 1603- |
| | 1612 (2010). |
| Bacillus Subtilis, SL4 | The Rockefeller University |
| | Bacteria Collection |
| Enterococcus faecalis, V12 | The Rockefeller University |
| | Bacteria Collection |
| Enterococcus faecium, EFSK-2 | The Rockefeller University, |
| | New York, NY. |
| Escherichia coli, DH5α | Invitrogen |
| Staphylococcus aureus, NRS105, | NARSA |
| Wood 46, (MSSA, protein A negative) | |
| Staphylococcus aureus, Newman | Kontermann, R. E. mAbs 4, |
| (MSSA) | 182-197 (2012) |
| Staphylococcus aureus, NRS623, | NARSA |
| RN4220/pCN57 (MSSA, constitutive | |
| GFP expression) | |
| Staphylococcus aureus, Newman/ | This disclosure |
| pCN57 (MSSA, constitutive | |
| GFP expression) | |
| Staphylococcus aureus, NRS382, | NARSA |
| USA100 (MRSA) | |
| Staphylococcus aureus, NRS383, | NARSA |
| USA200 (MRSA) | |
| Staphylococcus aureus, NRS384, | NARSA |
| USA300 (MRSA) | |
| Staphylococcus aureus, MW2, | Kontermann, R. E. mAbs 4, |
| USA400 (MRSA) | 182-197 (2012) |
| Staphylococcus aureus, NRS385, | NARSA |
| USA500 (MRSA) | |
| Staphylococcus aureus, NRS22, | NARSA |
| USA600, HIP07930 (VISA) | |
| Staphylococcus aureus, NRS386, | NARSA |
| USA700 (MRSA) | |
| Staphylococcus aureus, NRS387, | NARSA |
| USA800 (MRSA) | |
| Staphylococcus aureus, NRS1, | NARSA |
| Mu50, (VISA) | |
| Staphylococcus aureus, VRS2, | NARSA |
| HIP11983 (VRSA) | |
| Staphylococcus aureus, VRS3a, | NARSA |
| HIP13170 (VRSA) | |
| Staphylococcus epidermidis, | ATCC |
| ATCC 12228 | |
| Staphylococcus hyicus, HER1048 | |
| Staphylococcus sciuri subsp. | Kontermann, R. E. mAbs 4, |
| sciuri, K1 | 182-197 (2012) |
| Staphylococcus simulans, TNK3 | Kontermann, R. E. mAbs 4, |
| | 182-197 (2012) |
| Streptococcus agalactiae 090R | The Rockefeller University |
| | Bacteria Collection |
| Streptococcus pyogenes, SF370 | The Rockefeller University |
| | Bacteria Collection |

Abbreviations: ATCC—American Type Culture Collection; NARSA—Network on Antimicrobial Resistance in Staphylococcus aureus; MSSA—Methicillin Sensitive Staphylococcus aureus; MRSA—Methicillin Resistant Staphylococcus aureus; VISA—Vancomycin Intermediate Staphylococcus aureus, VRSA—Vancomycin Resistant Staphylococcus aureus.

### Example 7

This Example provides non-limiting examples of lysibodies comprising a binding domain from a bacteriocin—lysostaphin, as well as the creation of additional lysibodies with a phage lysin binding domain, directed against S. aureus. These lysibodies bound a range of clinically important staphylococcal strains, fixed complement on staphylococci, and induced phagocytosis of S. aureus by macrophages and neutrophils. Lysostaphin-lysibody effectively protected mice in a kidney abscess model. These results further demonstrate that the lysibody approach is a reproducible approach to creating anti-bacterial antibodies.

Construction of S. aureus Specific Lysibodies

The general design of lysibodies in this Example is similar to that of the lysibodies described in the Examples above. The lysibodies in this example are referred to as Lysostaphin-lysibody, LysK-lysibody, PlySa4-lysibody, PlySa6-lysibody, PlySa7-lysibody, and PlySa32-lysibody.

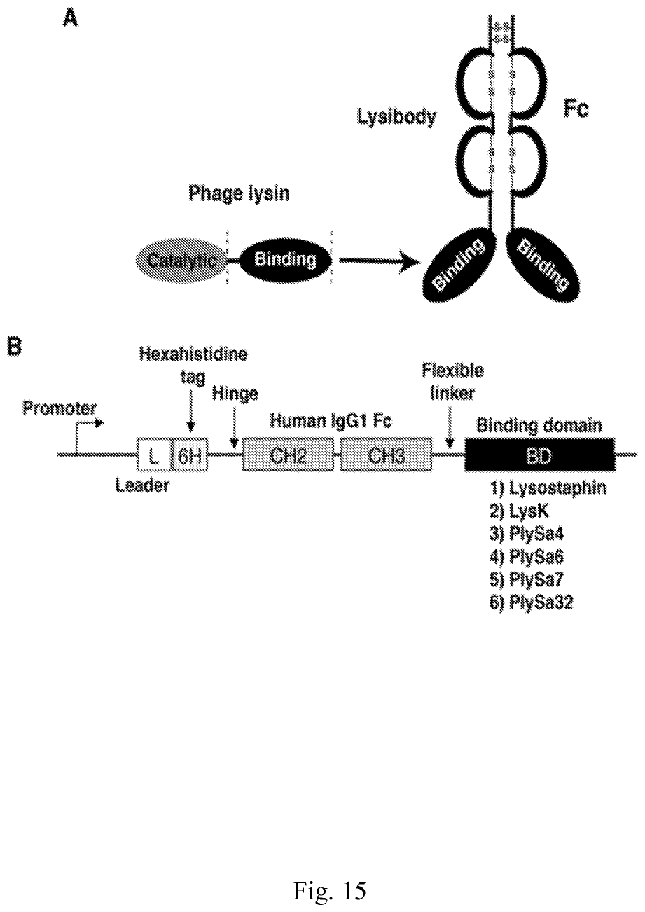

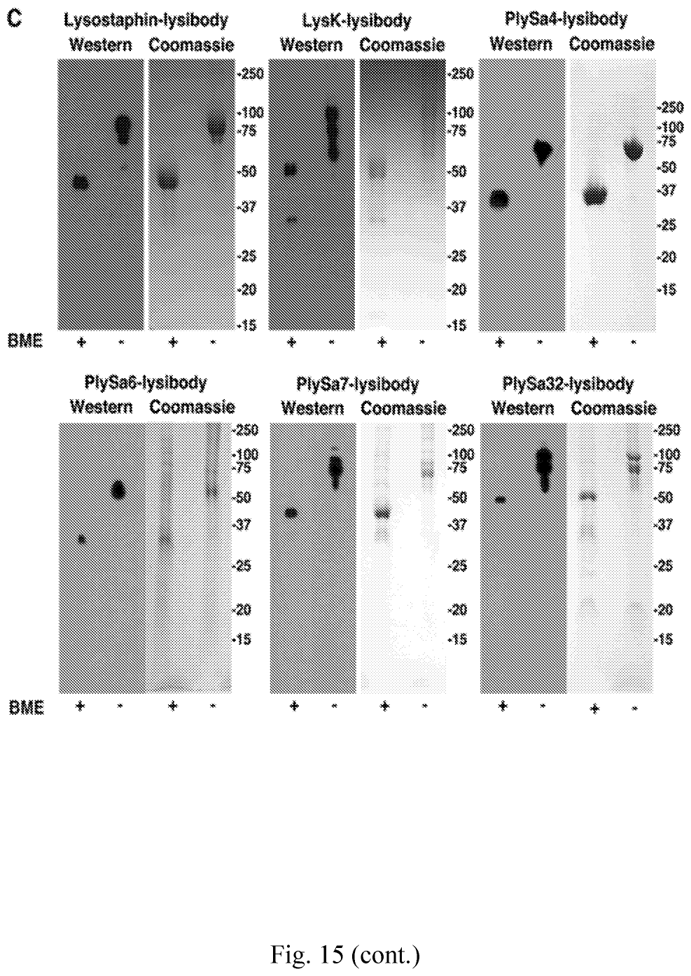

Lysibodies were expressed in 293T cells to allow correct glycosylation, and purified by metal affinity chromatography. Purified lysibodies were run on an SDS-PAGE in the presence or absence of β-mercaptoethanol (BME), which breaks the disulfide bonds between the subunits of lysibodies, similarly to native IgG antibodies. In the presence of BME all lysibodies displayed bands of a molecular weight compatible with the monomer, while in the absence of BME the bands were compatible with a dimer, demonstrating the proper formation of homodimers stabilized by disulfide bonds (FIG. 15).

### Characterization of Lysibody Activity

To analyze the functionality of the lysibodies described in this Example, we tested their ability to bind the cell wall of a protein A negative S. aureus strain Wood 46 using fluorescence microcopy (FIG. 16). All the S. aureus-specific lysibodies produced were able to bind the cell wall of this strain, while controls showed no binding.

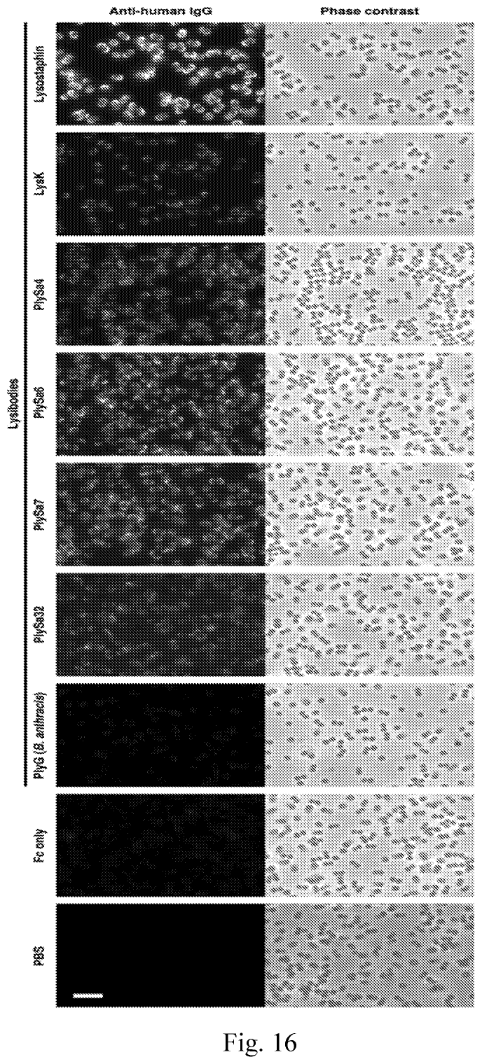

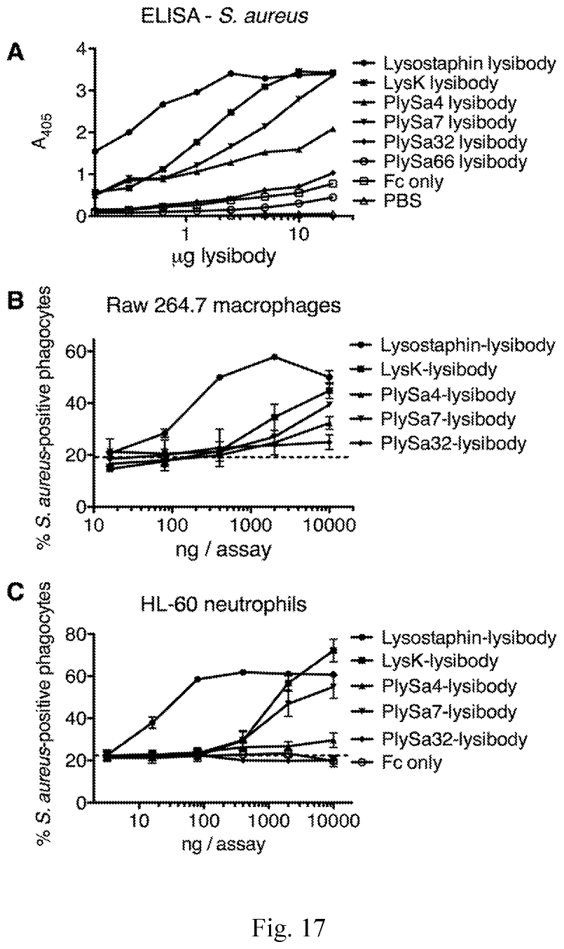

We then used a modified ELISA assay to compare the binding of different lysibodies to S. aureus. The protein A negative strain Wood 46 was immobilized on the bottom of a 96-well plate, fixed, and blocked. Lysibodies were serially diluted and incubated with the cells, and the plate was developed using an alkaline phosphatase conjugate. Lysostaphin lysibody showed the best binding, followed by LysK, PlySa7, and PlySa4 lysibodies (FIG. 17A). PlySa32 and PlySa66 showed only minor binding in this assay. Given the poor binding of PlySa66 and its very low yield following purification, this lysibody was not characterized further.

For the remaining five lysibodies, we performed in vitro characterization of their ability to induce phagocytosis of staphylococci by Raw 264.7 macrophage (FIG. 17B), and HL-60 neutrophils (FIG. 17C) using flow cytometry. In these assays as well, lysostaphin lysibody performed the best, followed by LysK, PlySa7, and PlySa4 lysibodies, whereas PlySa32 had little to no activity. Thus, the ELISA assay had a high predictive value for the ability of different lysibodies to perform in cellular phagocytosis assays.

### Microscopy

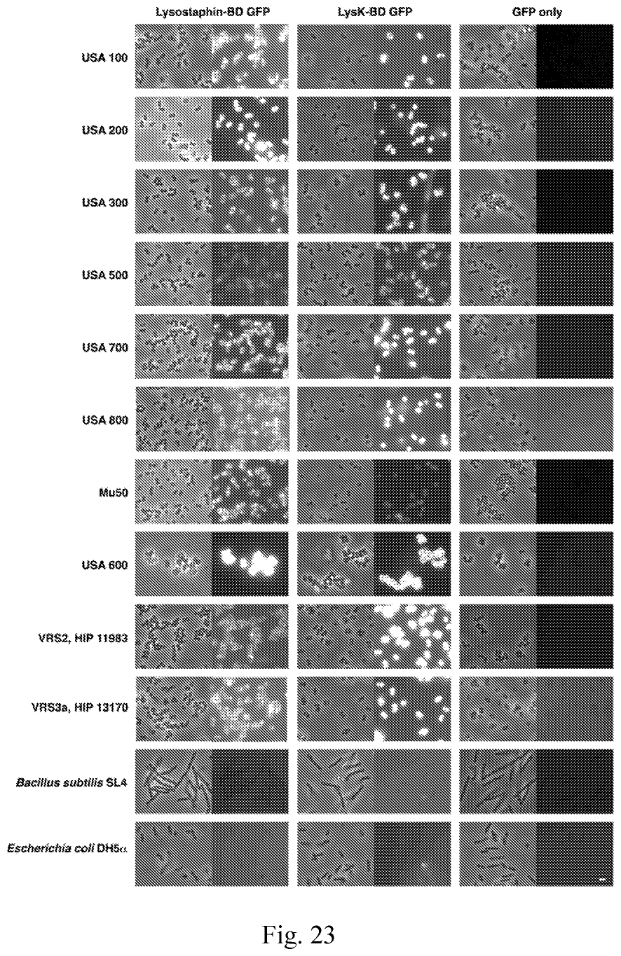

We used fluorescence microscopy to determine the range of clinically relevant staphylococcal strains that lysostaphin and LysK bind. GFP fusions to the binding domains of Lysostaphin and LysK were used to avoid potential non-specific fluorescent signal due to interaction of lysibodies and protein A on the surface of wild type S. aureus. The two GFP fusion proteins showed cell-wall specific labeling of all S. aureus strains tested including several clinically important methicillin and vancomycin resistant strains (FIG. 23). Neither construct bound to control organisms Bacillus subtilis, and Escherichia coli. GFP alone did not bind any of these strains.

For lysostaphin-lysibody, we also used a modified competitive ELISA to quantify the binding of lysostaphin lysibody to a range of bacterial strains. Different bacterial strains were grown overnight and brought to DO₆₀₀ values of 15, 10, 5, or 1 in PBS. Bacteria were mixed 1:1 with lysostaphin lysibody at 10 μg/ml for an hour. Following removal of bacterial cells by centrifugation, the amount of remaining lysibodies in the supernatant was determined by ELISA, using S. aureus protein A negative strain (FIG. 24).

This assay demonstrated that lysostaphin lysibody bound with high affinity to S. aureus as well as S. epidermidis, S. hyicus, and S. lugdunensis, and with somewhat lower affinity to S. sciuri. Little to no binding was observed for M. luteus, B. subtilis, B. anthracis, L. lactis, S. pyogenes, S. agalactiae, S. bovis/gallolyticus, E. faecalis, E. faecium, and E. coli, demonstrating high specificity for the genus Staphylococcus.

### Complement

We next determined the ability of the lysibodies to fix complement on the surface of S. aureus using immunofluorescence microscopy. S. aureus Wood 46 cells were incubated with lysibodies or controls, and then treated with complement. The extent of complement deposition on staphylococci was evaluated by fluorescence microscopy, using C3-specific antibodies and fluorescent conjugates. Lysostaphin and LysK lysibodies induced robust complement fixation on the surface of S. aureus while controls had no activity (FIG. 18).

Lysostaphin and lysK Lysibodies Induce Phagocytosis of S. aureus by Raw 264.7 Macrophage and Peritoneal Murine Macrophages

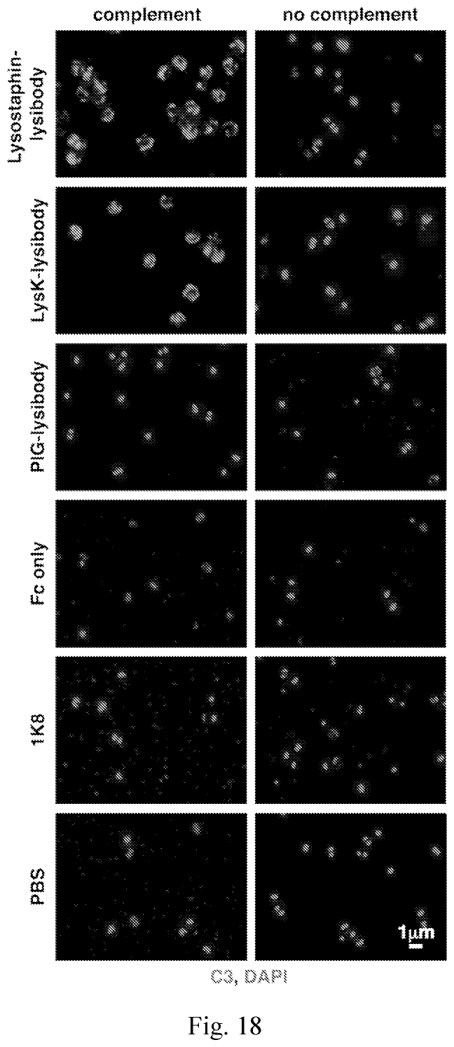

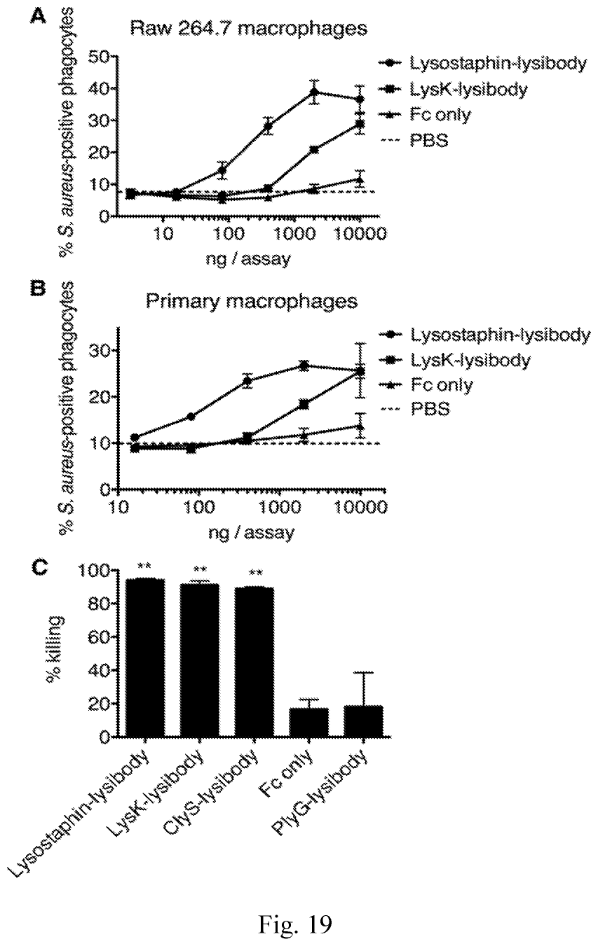

We expanded the macrophage phagocytosis data and compared the activity of lysostaphin and LysK lysibodies to that of the ClyS-lysibody. Lysostaphin-lysibody induced robust phagocytosis of S. aureus by Raw 264.7 macrophage, and was effective at lower doses compared to the previously characterized ClyS-lysibody (FIG. 19A). A higher concentration of LysK-lysibody was required to achieve maximum phagocytosis activity compared to the other two lysibodies.

Activity of lysostaphin-lysibody and LysK-lysibody was also determined using peritoneal murine macrophage, showing similar results to those obtained with Raw 264.7 macrophage (FIG. 19B). We also tested the ability of lysibodies to induce the killing of staphylococci by macrophages (FIG. 19C). Following 3 hours incubation with macrophages in suspension, lysostaphin-lysibodies, LysK-lysibody, induced the killing of over 90% of staphylococci, in line with the previously characterized ClyS-lysibody (FIG. 19C). PlyG-lysibody and Fc only controls did not lead to statistically significant killing of staphylococci.

### Neutrophils

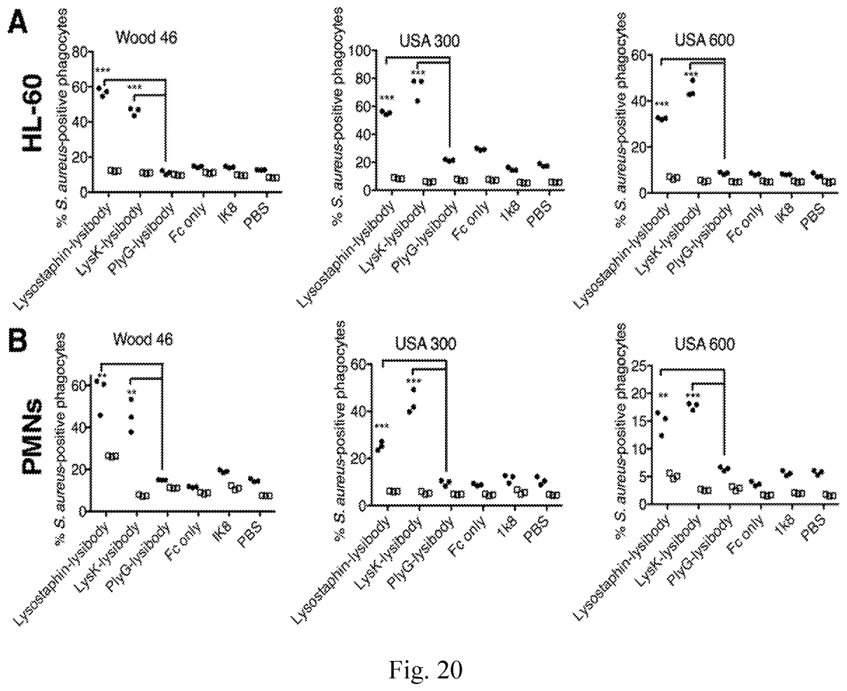

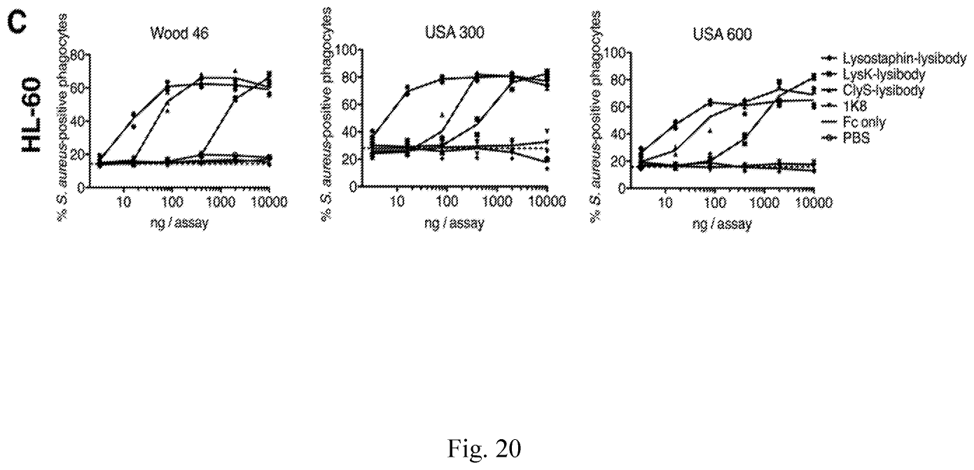

We next examined the ability of lysostaphin and LysK lysibodies to promote the phagocytosis of clinically important S. aureus strains (FIG. 20). When incubated with HL-60 neutrophils, lysostaphin and LysK lysibodies induced phagocytosis of S. aureus strains Wood 46 (MSSA, protein A negative), USA 300 (MRSA), and USA 600 (MRSA/VISA) in a complement-dependent manner, while controls had no activity (FIG. 20A). Similar results were obtained with human peripheral blood polymorphonuclear cells (PMNs). Both Lysostaphin-lysibody and LysK-Lysibody induced phagocytosis of all staphylococcal strains tested cells in a complement dependent manner (FIG. 20B). We next serially diluted the different lysibodies to determine the effective concentration for each lysibody, compared to the previously described ClyS-lysibody (FIG. 20C). With all tested strains, lysostaphin-lysibody was able to induce phagocytosis of staphylococci at the lowest concentration, followed by ClyS-lysibody, and LysK-lysibody. At a high concentration however LysK-lysibody was in some cases able to induce more efficient phagocytosis than Lysostaphin-lysibody, as is also apparent in FIG. 20 panels A and B.

### Mouse Protection

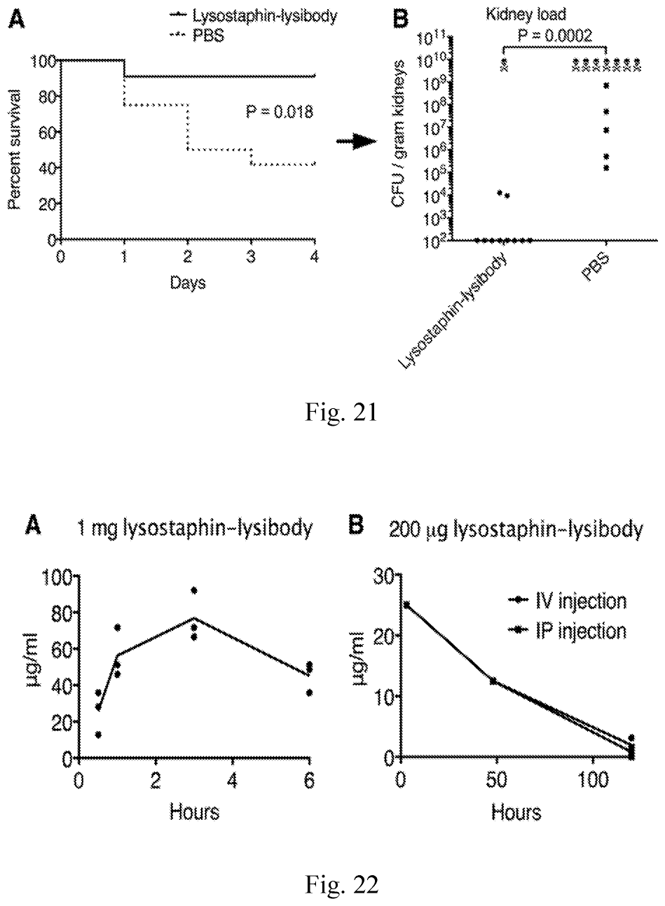

We further tested the ability of Lysostaphin-lysibody to protect mice from a challenge with MRSA/VISA strain USA600. Mice were injected intraperitoneally with 1 mg Lysostaphin-lysibody, and 4 hours later challenged intraperitoneally with 5×10⁶ S. aureus USA600. Mice viability was monitored daily (FIG. 21A). After 4 days surviving mice were sacrificed, and the bacterial load in the kidneys was evaluated (FIG. 21B). Mice treated with lysostaphin-lysibody had improved survival (91%) compared to control mice (42%). Furthermore, 8 of the 10 surviving lysostaphin-lysibody treated mice had no detectable bacteria in their kidneys whereas all surviving control mice had bacteria in their kidneys ranging between 10⁵-10⁹ CFU/g.

### Pharmacokinetics

Next, we determined the rate of lysibody clearance from mouse blood. In one experiment we examined lysibody concentration in the blood during the first few hours following injection of 1 mg lysostaphin-lysibody IP. Lysibody concentration increased first few hours following injection, reaching a peak around 3 hours following injection, and then begun to decline (FIG. 22A). We then tested the decline rate over a 5-day period. Four mice were each injected with 200 μg lysostaphin-lysibody. Of these two were injected IP and two were injected IV thorough the tail vein. Both methods of injection resulted in a similar initial concentration in the blood. Lysibody concentration in the blood dropped from an average of around 25 μg/ml to around 12 μg/ml in the first 48 h, and then declined to around 1.4 μg/ml after 120 hours.

It will be apparent from the foregoing that this Example describes creation of six lysibodies utilizing binding domains from lysostaphin, LysK, PlySa4, PlySa6, PlySa7, and PlySa32, all of which bound to their target organism. Lysostaphin and lysK lysibodies were analyzed further based on their strong binding to S. aureus wall, high potency in inducing phagocytosis of S. aureus, and good expression level. Additional analysis of the lysostaphin and LysK lysibodies showed that both were capable of fixing complement on the surface of S. aureus, and induce phagocytosis of S. aureus by macrophages and neutrophils. Lysostaphin lysibody, which demonstrated superior activity in in vitro phagocytosis assays, was tested in a mouse model, and protected mice from a challenge with MRSA/VISA strain of S. aureus.

Of the lysibodies described in this Example, lysostaphin lysibody was the most active. Lysostaphin is a bacteriocin, produced by Staphylococcus simulans biovar staphylolyticus that cleaves the pentaglycine cross bridge found in the cell wall of S. aureus and related organisms, as a means to control its environmental niche. It can kill both dividing and non-dividing staphylococci. It targets S. aureus and coagulase negative staphylococci, however coagulase-negative staphylococci are generally less sensitive to the lytic activity of staphylococci, and displayed variation in their ability to be lysed by lysostaphin. It is composed of an N-terminal catalytic domain and a C-terminal binding domain, and the mature form is 246 amino acids, and a molecular weight of 25 kDa.

### The Binding and Activity Range of Lysostaphin

Lysostaphin is generally active against staphylococcal clinical isolates. In one study 429 isolates were tested for lysostaphin sensitivity using disc diffusion, and all proved sensitive. In a different study lysostaphin was able to lyse all of the 257 isolates tested, including 168 MSSA and 89 MRSA strains. Lysostaphin was shown active against MRSA methicillin resistant coagulase negative staphylococci, as well and vancomycin resistant S. aureus. These results are in line with our observed ability of lysostaphin lysibody to bind a range of clinical isolates including methicillin and vancomycin resistant strains, as well as coagulase-negative staphylococci.

### Pharmacokinetics

Our finding was that the pharmacokinetics of the lysostaphin lysibody was about a two days half-life. This is at the lower range of what is typically observed for monoclonal antibodies, however it is significantly longer that the half-life observed for many lysin molecules, which could be as low as 20 minutes.

The following materials and methods were used to obtain the results described in this Example.

### Methods

### Cell Lines, Bacteria, and Media

Cell line 293T was from the lab of Michel Nussenzweig at Rockefeller University. Cells were grown in Dulbecco's Modified Eagle Medium (DMEM), 10% heat-inactivated FBS (Sigma), 2 mM sodium pyruvate (Sigma). HL-60 (ATCC number: CCL241), were propagated in RPMI 1640 (Gibco, Life Technologies), 10% heat inactivated FBS, GlutaMax (Gibco, Life Technologies), with penicillin and streptomycin. HL-60 differentiation was performed using known approaches. Raw 264.7 murine macrophage (ATCC: TIB-71), were grown in minimum essential media (MEM, Gibco, Life technologies), 10% heat inactivated FBS, 1 mM sodium pyruvate. Tissue culture cells were incubated at 37° C., 5% CO₂. Cells lines were demonstrated clear of mycoplasma using MycoAlert PLUS Assay (Lonza), at Memorial Sloan Kettering Cancer Center Antibody and Bioresource Core Facility.

The source of bacterial strains used in this study is presented in Table 2. Streptococcal strains were grown stationary in Todd-Hewitt medium (Difco) containing 1% yeast extract (Fisher Scientific), stationary at 37° C. Enterococcal strains were grown in brain heart infusion (BHI), stationary at 37° C. Staphylococcal strains were grown in BHI at 37° C. with shaking. Escherichia coli strains were grown in LB medium at 37° C. with shaking.

### Reagents

Goat anti-human IgG (gamma chain specific) alkaline phosphatase conjugate (Sigma, A3187) was diluted 1:5000. Polyclonal anti-human C3c complement, produced in rabbit (Dako) was diluted 1:500 for microscopy and 1:2000 for flow cytometry.

Goat anti-Human IgG Fcγ fragment, DyLight 594 conjugate (Jackson ImmunoResearch,) was diluted 1:1000. Goat anti-rabbit IgG Alexa Fluor 594 conjugate (Life Technologies) was diluted 1:1000 for microscopy and 1:2000 for flow cytometry. Goat serum was from Sigma. Wheat germ agglutinin (WGA) conjugates to Alexa Fluor 594, and Alexa Fluor 488 (Molecular Probes) were used at 5 μg/ml. DAPI (Sigma) was used at 1 μg/ml. Fluorescein isothiocyanate (FITC, Sigma), and NHS-Rhodamine red (Thermo Scientific), were used according to manufacturer instructions. Dulbecco's phosphate buffered saline (DPBS/Modified) with CaCl₂) and MgCl₂ was from HyClone. DPBS without CaCl₂) and MgCl₂ was from Gibco. Other reagents were from Sigma unless otherwise noted.

### Construction of the Plasmids

For the creation of expression vectors for the various lysibodies, PCR products encompassing the binding domains of the various cell wall hydrolases were produced, and inserted into the SalI and HinDIII sites of pAR422_ClyS-lysibody (ref) (replacing the binding domain found in this plasmid) as follows:

The binding domains of lysostaphin were amplified using primers Lysostaphin-BD_5_SalI_564 and Lysostaphin-BD3_HinDIII_565, and used to create plasmid pAR500_lysostaohin-lysibody.

The binding domain of LysK was amplified using primers LysK-BD_5_SalI_584 and LysK-BD_3_HinDIII_585, and used to create plasmid pAR501_LysK-lysibody.

A binding domain with LysM homology (Reference Sequence: WP_037588827.1) found in the genome of S. aureus C0673 was amplified using primers PlySa4-BD_5_SalI_566 and PlySa4-BD_3_HinDIII_567, and used to create plasmid pAR502PlySa4-lysibody.

The binding domain of a phage lysin encoded in ORF009 of staphylococcal phage 66 (Reference Sequence: YP_239469.1) was amplified using primers PlySa6-BD_S_SalI_572, and PlySa6-BD 3 HinDIII_573, and used to create plasmid pAR503 PlySa6-lysibody.

The binding domain of an amidase (Reference Sequence: WP_031863627.1) from S. aureus genomic scaffold 2011-60-1490-31 adZCS-supercont1.41 was amplified using primers PlySa7-BD_5_SalI_574 and PlySa7-BD 3 HinDIII_575, and used to create plasmid pAR504_PlySa7-lysibody.

The binding domain of an amidase (Reference Sequence: WP_001140246.1) from S. aureus genomic DNA was amplified using primers PlySa32-BD_5_SalI_576 PlySa32-BD_3_HindIII_577, and used to create plasmid pAR505_PlySa32-lysibody.

GFP—lysin binding domain constructs were produced as follows: The binding domain of Lysostaphin was amplified using primers Lysostaphin-BD_5 XbaI_588 and Lysostaphin-BD_3 PstI 589, and inserted into the XbaI and PstI sites of pAR159, yielding pAR519_lysostaphin-BD-GFP

The binding domain of LysK was amplified using primers LysK-BD_5_XbaI_590 and LysK-BD_3_PstI_591, and inserted into the XbaI and PstI sites of pAR159, yielding pAR520_LysK-BD-GFP.

### Expression and Purification of Lysibodies

Lysibody expression vectors were transfected into 293T cells using the PEI method and FreeStyle 293 Expression Medium (Life Technologies). Lysibodies were purified from the culture supernatant using metal affinity chromatography as described above.

### Fluorescence Microscopy

Fluorescence microscopy was performed essentially as previously described above. In brief, bacteria were fixed using 2.6% paraformaldehyde, 0.012% glutaraldehyde, in growth medium containing 30 mM phosphate buffer pH 7.4 for 15 min at room temperature, and 30 min on ice. Bacteria were washed, attached to poly-L-lysine coated cover glass, and blocked with 10% normal goat serum. Bacteria were incubated with lysibodies at 2 μg/ml in PBS containing 2% BSA and 1% gelatin, and then fluorescent conjugates diluted 1:1000, each for 1 h at room temperature. Slides were mounted in 50% glycerol and 0.1% p-phenylenediamine in PBS pH 8.

Phase-contrast microscopy and fluorescent microscopy were performed using a Nikon Eclipse E400 microscope, equipped with a Nikon 100×/1.25 oil immersion lens, and a Retiga EXi fast 1394 camera (QImaging). Image capture was done using QCapture Pro version 5.1.1.14 software (QImaging). Deconvolution microscopy was done on a DeltaVision image restoration microscope (Applied Precision/Olympus) equipped with CoolSnap QE cooled CCD camera (Photometrics). An Olympus 100×/1.40 NA, UPLS Apo oil immersion objective was used with a 1.5× optovar. Z-stacks intervals were 0.15 m. SoftWoRx software (Applied Precision/DeltaVision) was used for image deconvolution, and resulting images were corrected for chromatic aberrations.

### Phagocytosis Assays

Phagocytosis assays were performed essentially as described above. In brief, 24-well plates containing confluent Raw 264.7 macrophages or peritoneal macrophages, derived from BALB/c female mice, were supplemented with 1×10⁷ S. aureus Newman/pCN57 (expressing GFP), and incubated for 1 h at 37° C. 5% CO₂ at various lysibody concentrations. The wells were washed several times to remove extracellular bacteria, and fixed with 1 ml 1% paraformaldehyde in PBS for 1 h at 4° C., and washed. Raw 264.7 macrophages were scraped off the plate using a disposable loop in 200 μl PBS, while peritoneal macrophages were incubated with 250 μl 0.25% trypsin in PBS pH 7.2 0.1% EDTA for 30 min at 37° C., and then gently suspended by pipetting with a 1 ml pipette tip, and washed.

For killing experiments, each assay contained 10⁵ log-phase S. aureus strain Newman cells, and 10 μg lysibody in a total volume of 100 μl HBSS 0.1% gelatin, in a well of a 96-well U-bottom plate. The plate was placed of a shaker for 1 h at 200 RPM 4 C° . 10⁵ Raw 264.7 macrophages were then added in a total volume of 100 μl HBSS (200 μl final volume), and the plate was placed on a shaker for 3 h at 200 RPM 37 C°. Samples were lysed in 0.2% saponin, serially diluted in distilled water, and plated for CFU quantification. Experiments were performed in triplicates, and included three technical duplicates for each biological repeat.

For phagocytosis assays with neutrophils, HL-60 neutrophils were prepared using known approaches, and human peripheral blood PMNs were isolated from health volunteers are previously described (ref my paper). Neutrophils were suspended in Hanks' balanced salt solution (HBSS, Gibco 14025) containing 0.1% gelatin at 1×10⁸ cells/ml. FITC-labeled bacteria were suspended to a final of 5×107 cells/ml in HBSS 0.1% gelatin. 10 μl bacteria were added to each well of a U-bottomed 96-well plate containing 60 μl HBSS 0.1% gelatin and lysibodies. Following 1 h incubation with shaking at 4° C., 10 pal of 5% S. aureus-adsorbed complement, and 10 μl neutrophils at 1×10⁸ cells/ml were added, and the plate was shaken at 200 RPM at 37° C. for 1 h. The cells were then fixes with 2.6% paraformaldehyde for 1 h, blocked, and analyzed by flow cytometry, first gating on neutrophils using forward and side scatter, and then determining the percentage of neutrophils containing fluorescent S. aureus as previously described (ref).

### Complement Fixation

Complement fixation procedures were performed essentially as previously described. Briefly, for microscopy experiments staphylococci were attached to poly-L-lysine coated cover slides and incubate with lysibodies at a final concentration of 1 mg/ml in DPBS for 1 h at room temperature. The cells were washed with PBS and

DGHB (5 mM HEPES, 71 mM NaCl, 0.15 mM CaCl₂, 0.5 mM MgCl₂, 2.5% glucose, 0.1% gelatin, pH 7.4), and then incubated for 20 minutes at 37° C. with 30 μl DGHB containing 0.5% S. aureus-adsorbed human serum. The cells were washed with PBS and fixed with 2.6% paraformaldehyde in PBS for 1 h at 4° C. The cells were PBS, and blocked with PBS 2% BSA 1% gelatin. C3b deposition was detected with rabbit anti-C3 diluted 1:500, followed by goat anti-rabbit Alexa Fluor 594 conjugate diluted 1:1000 and 1 μg/ml DAPI.

### Complement Fixation on Newman and Flow Cytometry

For microscopy on Newman/pCN57 (Protein A positive, expressing cytoplasmic GFP), the cells were blocked following fixation using PBS 2% BSA 1% gelatin, followed by heat inactivated goat and human sera.

S. aureus Newman/pCN57 were grown on a BHI plate for one day at 37° C. and for another day at 25° C. Several separate colonies were scraped off the plates and suspended in PBS to a final OD₆₀₀ 1.0. 30 μl bacteria were mixed with lysibodies or controls at various concentrations, and the final volume was adjusted to 200 μl with PBS. The cells were rotated at 4° C. for two hours, and washed with 0.5 ml saline and then 100 μl GVB (gelatin veronal buffer, Sigma). The cells were suspended in 300 μl 3% human complement (adsorbed on S. aureus, see above) in GVB, and the tubes were rotated at 37° C. for 15 min. The samples were then immediately placed on ice, and EDTA was added to a final concentration of 20 mM to stop complement fixation. The samples were washed twice with PBS, and fixed with 250 μl 2.6% paraformaldehyde in PBS (phosphate adjusted to 40 mM, pH 7.4) for 1 h at 4° C. The samples were then washed twice with PBS, and the pellet was blocked with 100 μl PBS 2% BSA 1% gelatin for 20 min. 100 μl 10% heat-inactivated goat serum were added for an additional 20 minutes, and then 10 μl heat-inactivated human serum were added to each sample for an additional 20 min, in order to block protein A. The cells were then washed with PBS, and each tube was suspended in 100 μl rabbit anti C3 antibody diluted 1:2000 in PBS 2% BSA 1% gelatin, and rotated for 1 h at room temperature. The cells were washed with PBS, suspended in 100 μl goat anti rabbit Alexa Fluor 594 conjugate diluted 1:2000 in PBS 2% BSA 1% gelatin, and the tubes were rotated for 1 h at room temperature. The cells were then washed with PBS and resuspended in 200 μl PBS. Samples were analyzed using a BD-Accuri C6 flow cytometer, and the CFlow and FlowJo softwares. Unlabeled and mono-labeled samples were used to calibrate compensation values. Gating was done on GFP-positive cells to exclude non-S. aureus particles, and C3b signal distribution was determined.

### ELISA Assays

Plate preparation pas performed as follows: High binding polystyrene 96-well plate were coated with 100 μl 0.01% poly-L-lysine at room temperature for 1 h and washed with water. S. aureus strain Wood 46 (protein A negative) was grown overnights, washed, and resuspended in PBS to a final OD₆₀₀ of 1.0. To each well of the microtiter plate were added 50 μl cell suspension and 50 μl freshly prepared 3.2% paraformaldehyde solution in PBS. The plates were incubated at room temperature for 20 min, and centrifuged for 20 min at 1500 g, 4° C. The plates were washed with water three times, and incubated with 150 l/well 0.1M lysine in PBS for 1 h at room temperature. The plates were then washed three times with water and twice with ELISA wash buffer (10 mM sodium phosphate, 150 mM NaCl, 0.05% Brij-35, 0.02% sodium azide). The plates were then blocked with PBS 1% BSA overnight at 4° C.

For ELISA to compare the binding of different lysibodies to S. aureus various lysibodies were serially diluted two-fold from an initial stock of 10 μg/ml, and 50 μl of each dilution was transferred to a well of the prepared ELISA plate, and incubated overnight at 4° C. The wells were then washed three times with water and twice with wash buffer, and incubated with 100 μl goat anti-human IgG Fc alkaline phosphatase conjugate at 1:10,000 dilution for 3 h at 37° C. The plate was washed as above, developed with p-nitrophenyl phosphate (pNPP), and absorption at 405 nm was measured using a SpectraMax Plus plate reader (Molecular Devices).

For studies involving lysibody adsorption of bacterial strains, each of the strains were grown overnight, washed, and suspended to a final calculated OD₆₀₀ 15, from which it was sub-diluted to OD₆₀₀ of 10, 5, and 1. In a U-bottomed 96-well plate that was pre-blocked with PBS 1% BSA, 50 μl of lysibody at 10 μg/ml were mixed with 50 μl of bacterial suspensions and incubated for 1 h at 37° C. with 200 RPM shaking. The plate was then centrifuged for 10 min at 4000 RPM, and 75 μl of the supernatant was transferred to an ELISA plate. Preparation and processing of the ELISA plate was performed as described above.

### Mouse Kidney Abscess Model

Mouse kidney abscess model was modified from the Examples above. Five weeks old BALB/c female mice (Charles River laboratories) were injected IP 1 mg lysibody or control. Four hours later 5×10⁶ CFU of S. aureus strain USA600 (MRSA, VISA) in saline 5% hog gastric mucin (Sigma) were injected to each mouse. Bacteria were prepared as follows: an overnight culture of USA600 was diluted 1:100 in BHI, and grown to OD₆₀₀ 0.5. at 37° C. with shaking at 200 RPM. Bacteria were washed in saline, suspended in saline to OD₆₀₀ 1.0, and diluted to the desired concentration; actual CFU/ml were evaluated by plating. Mouse were monitored daily, and after 4 days surviving mice were sacrificed, and the kidney bacterial load was determined by ascetically removing the kidneys, grinding in 1 ml 0.5% saponin, serial dilutions, and plating.

### Statistical Analysis

Two-tailed student's t-test was used to evaluate statistical significance in phagocytosis assays. For the mouse kidney abscess model, statistical significance for bacterial load in the kidneys was evaluated using the two-tailed Mann-Whitney test; the maximal bacterial load value observed in these experiments was assigned to mice that succumbed to infection. Prism version 5.0c (GraphPad Software, La Jolla, Calif.) was used for data analysis.

### Example 8

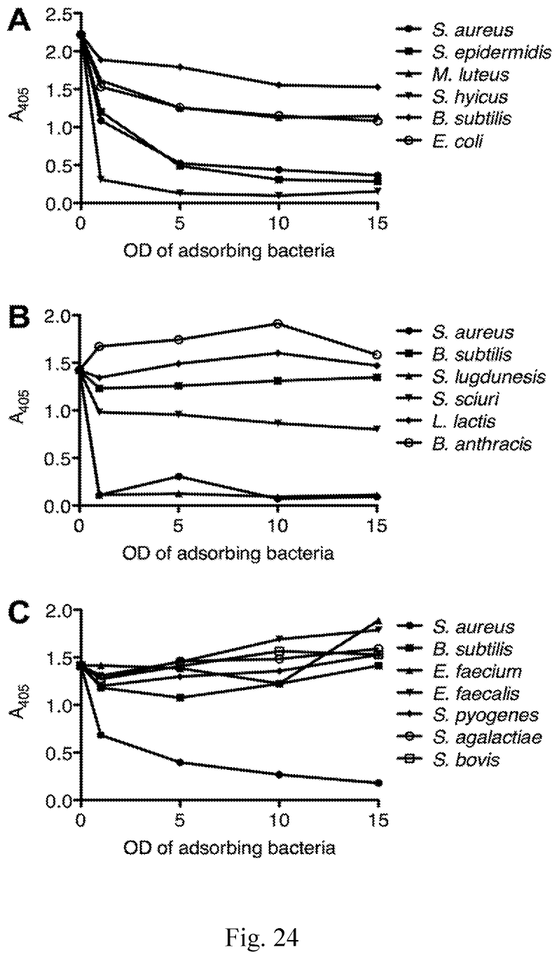

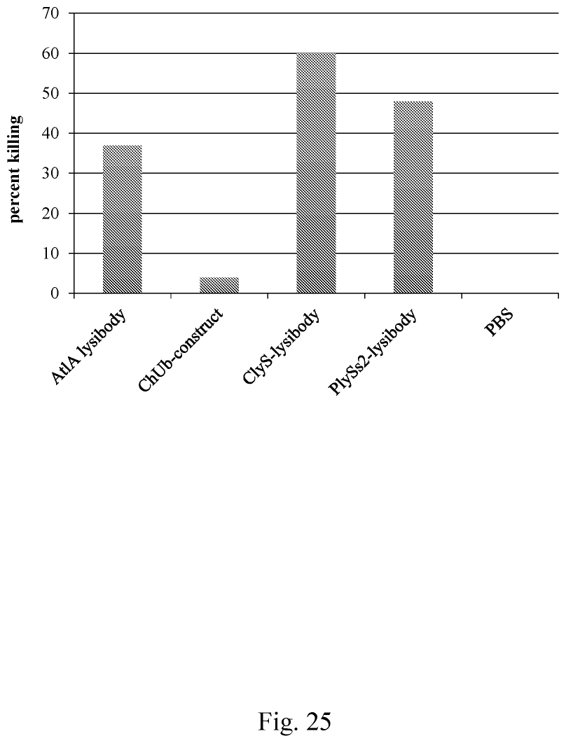

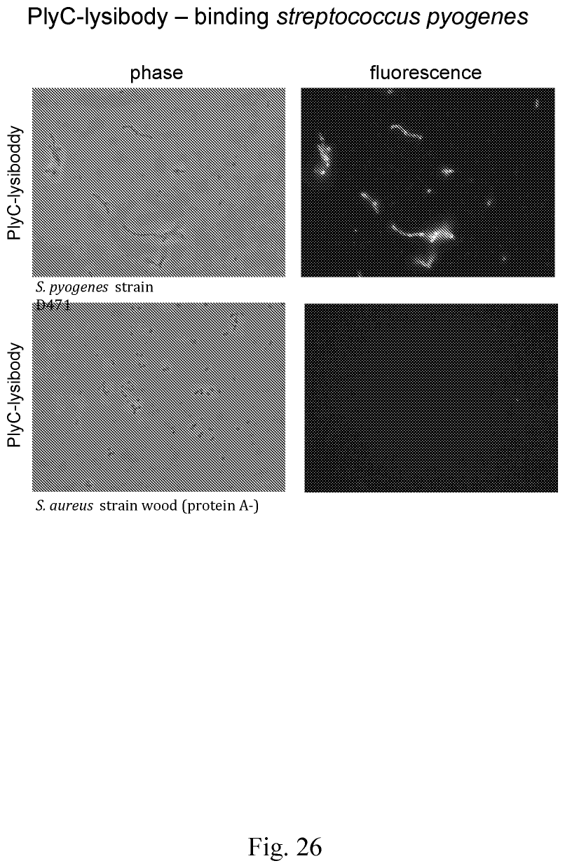

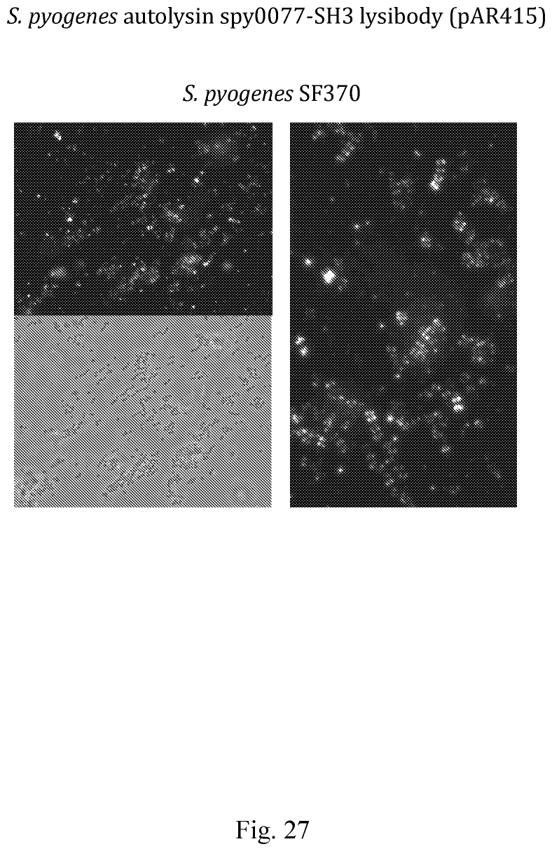

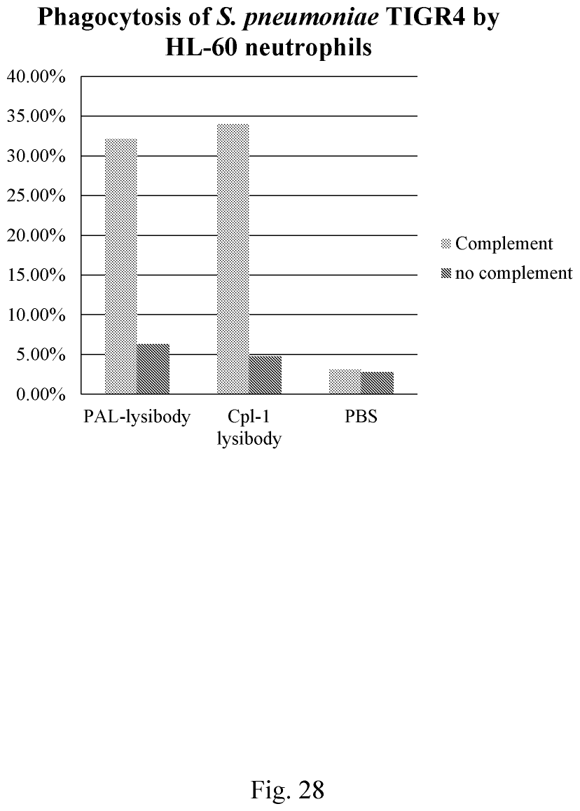

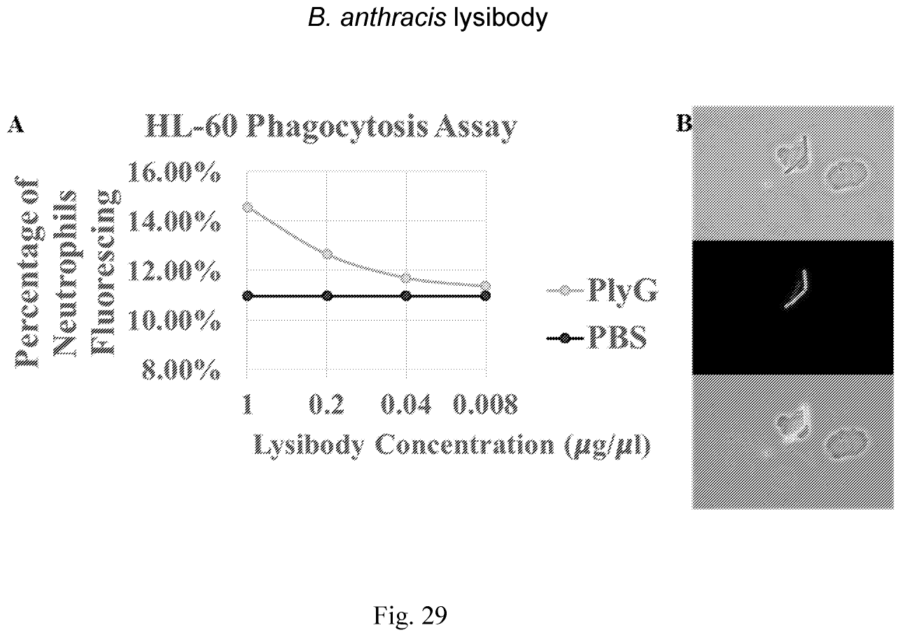

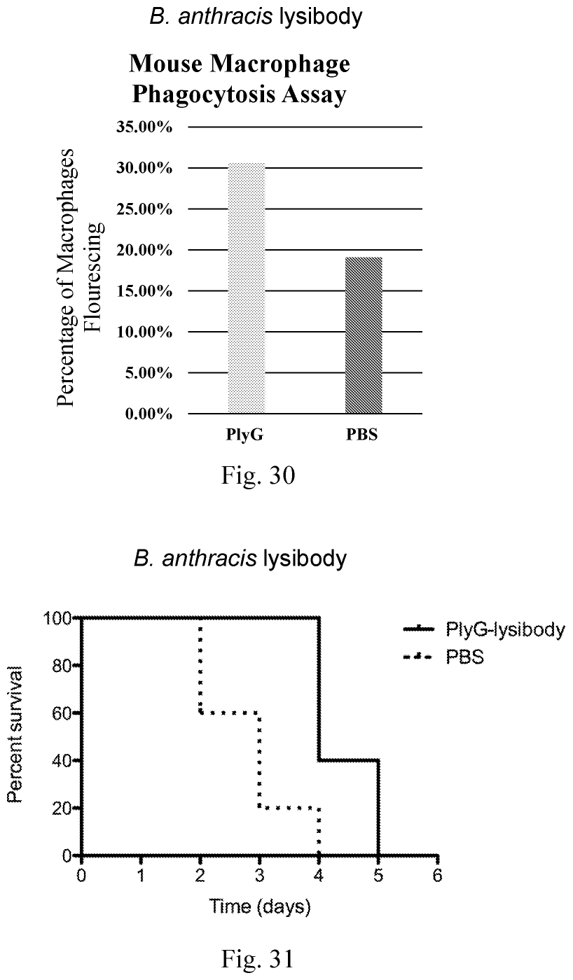

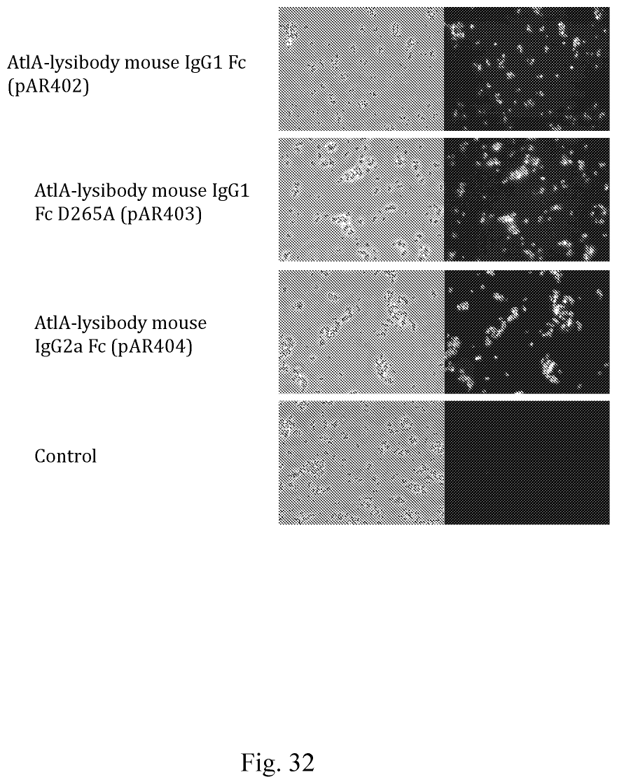

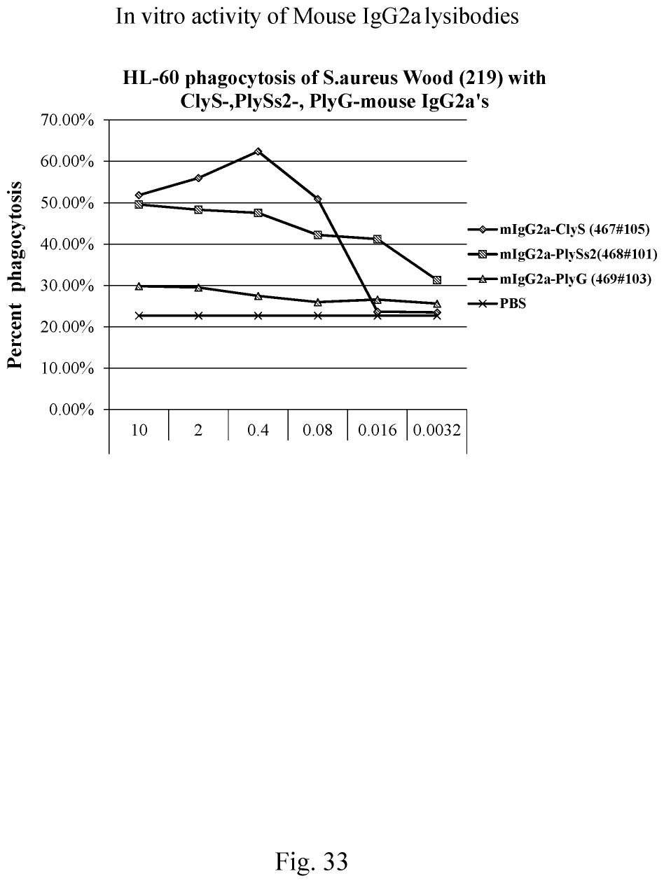

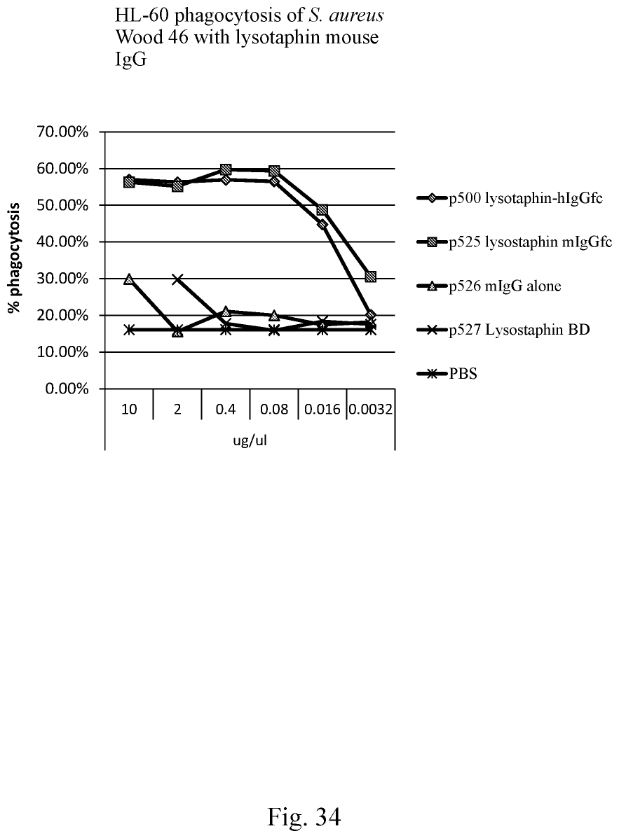

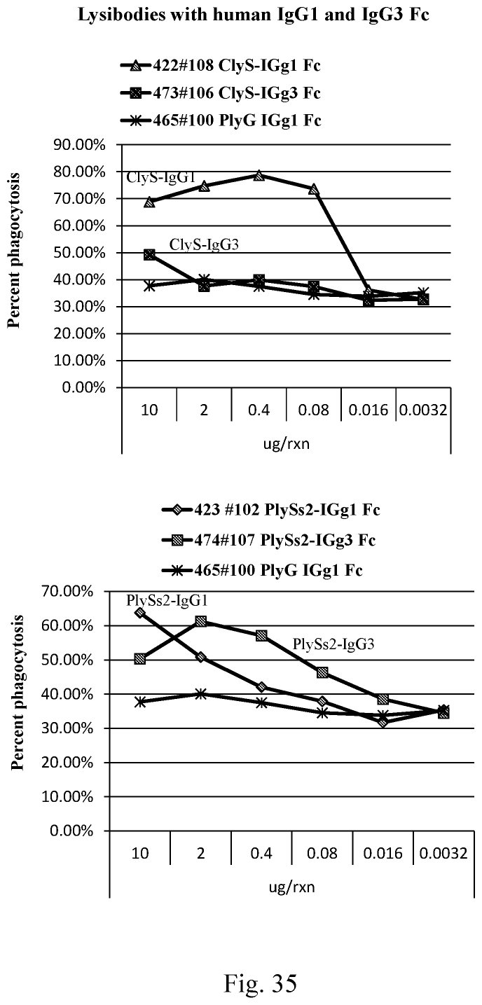

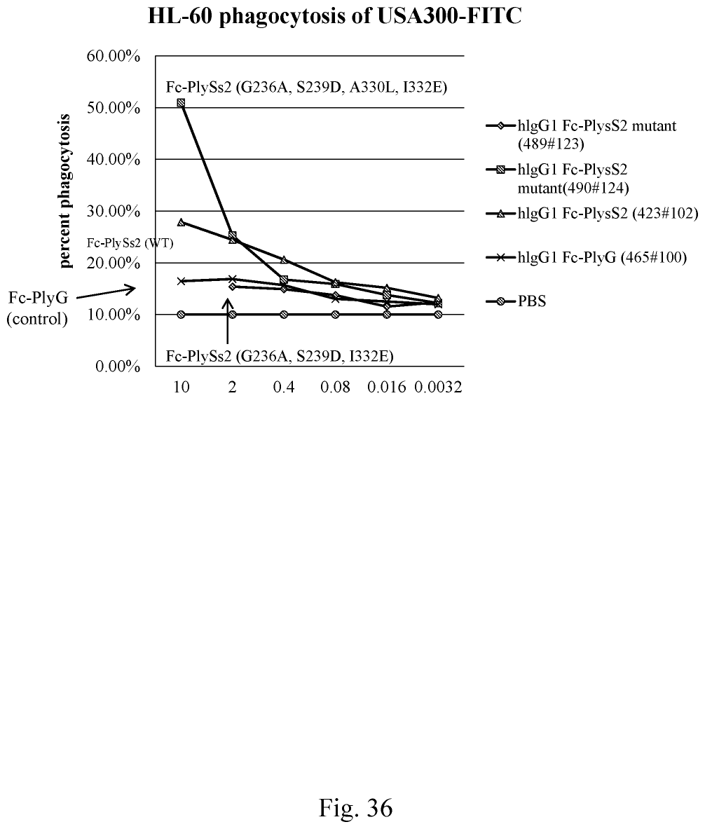

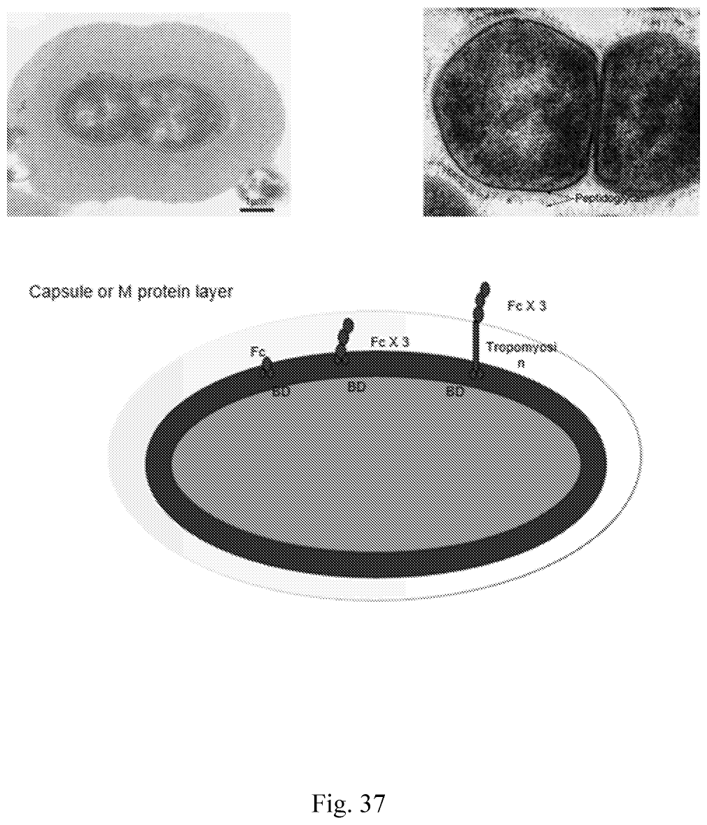

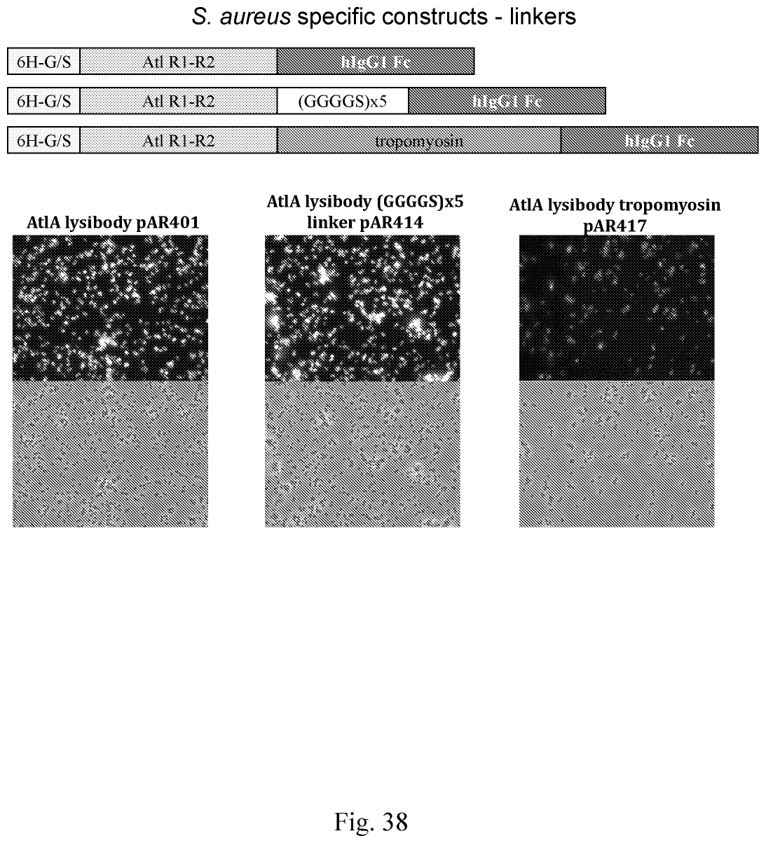

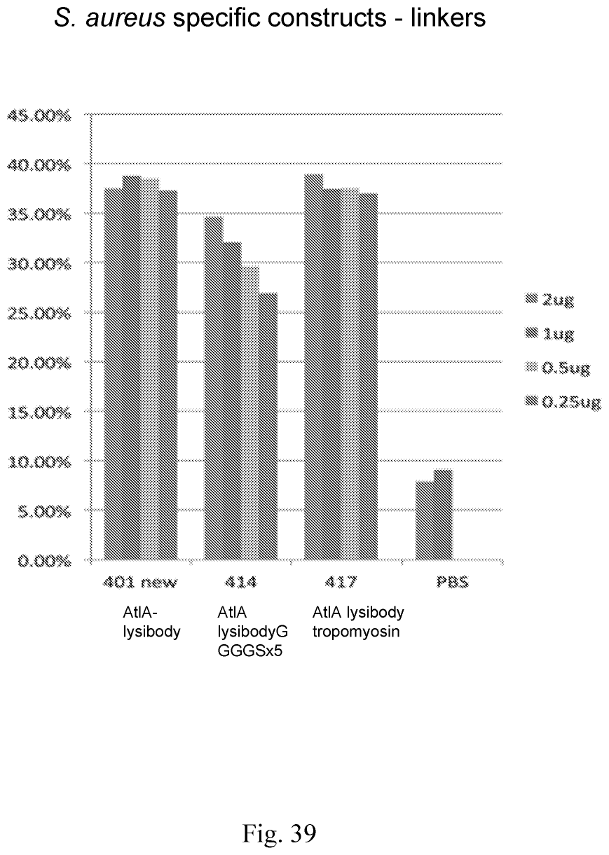

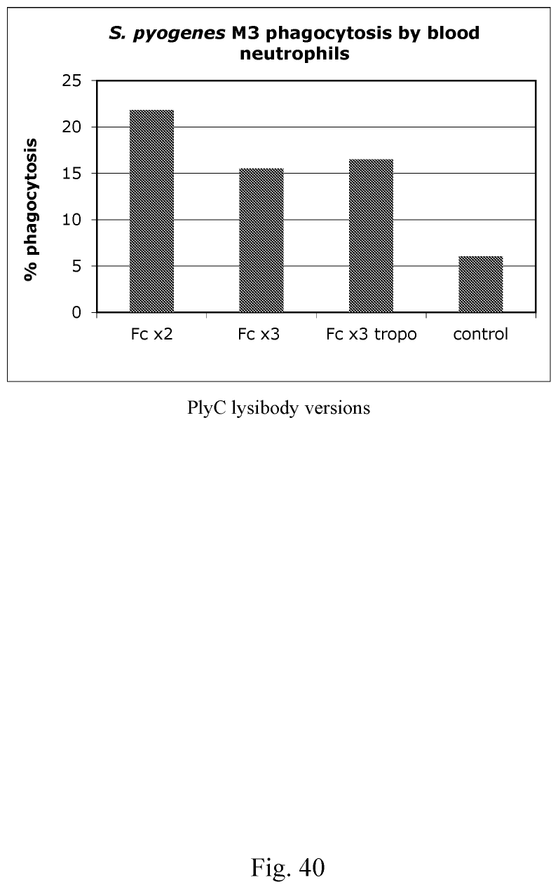

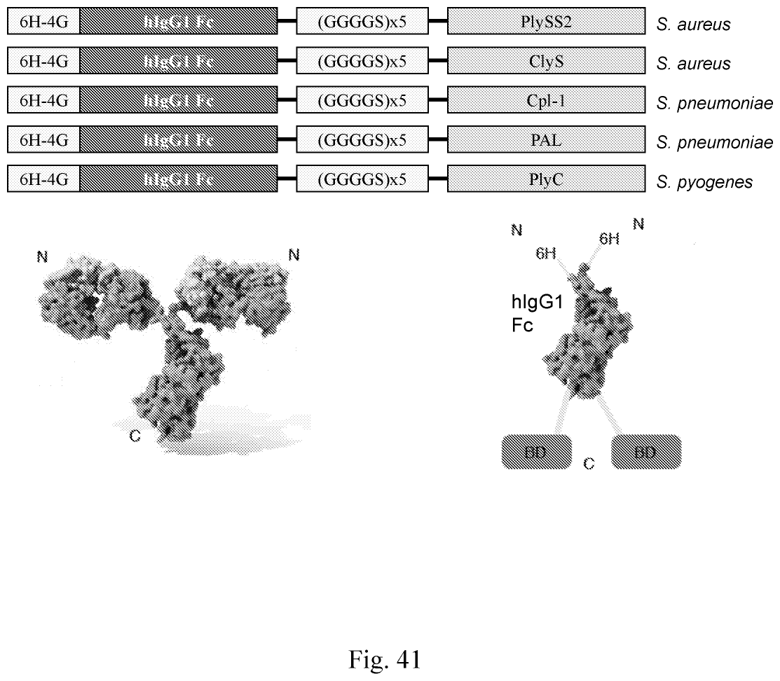

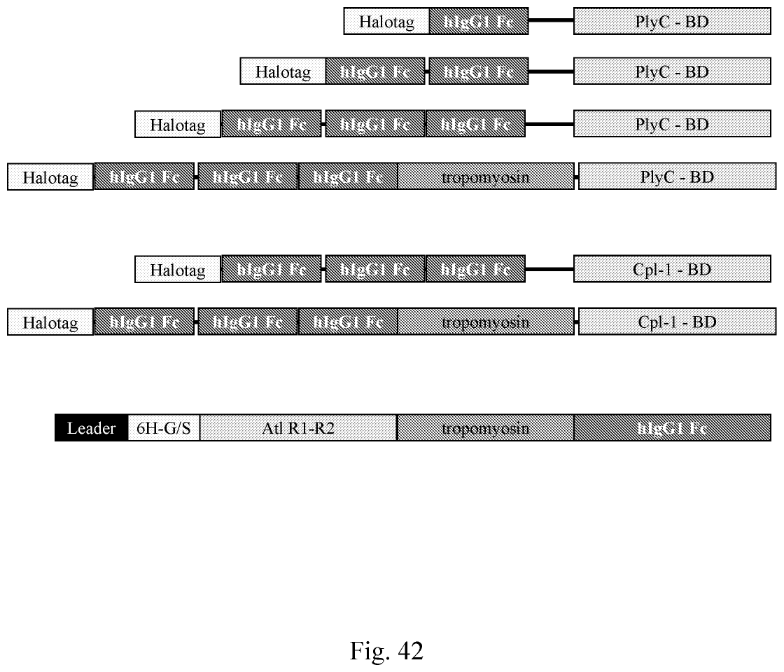

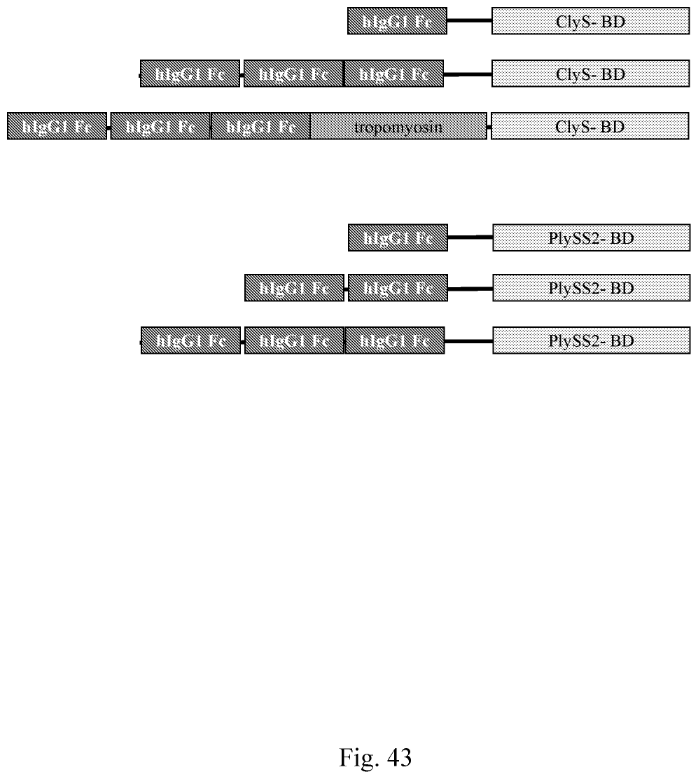

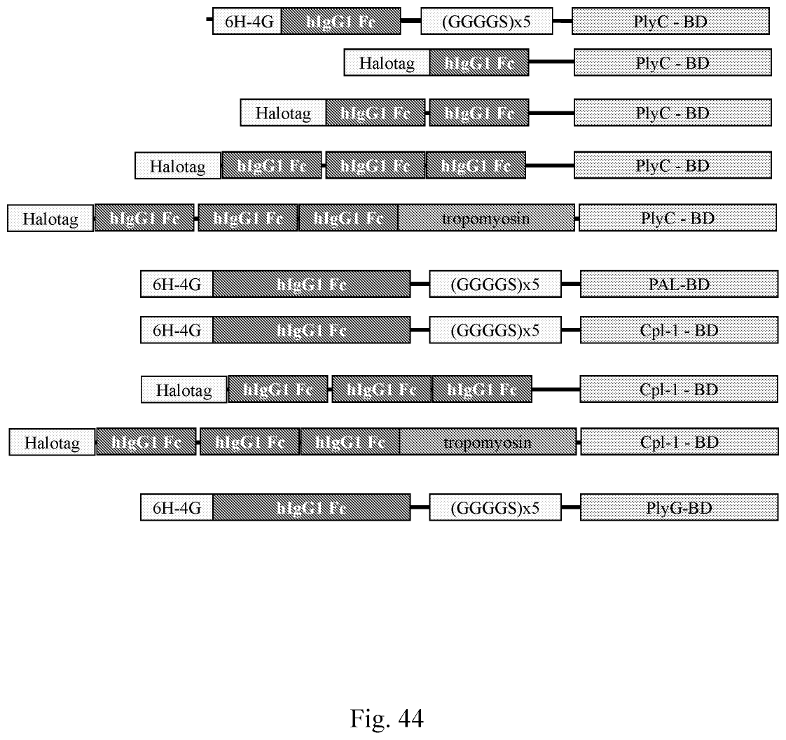

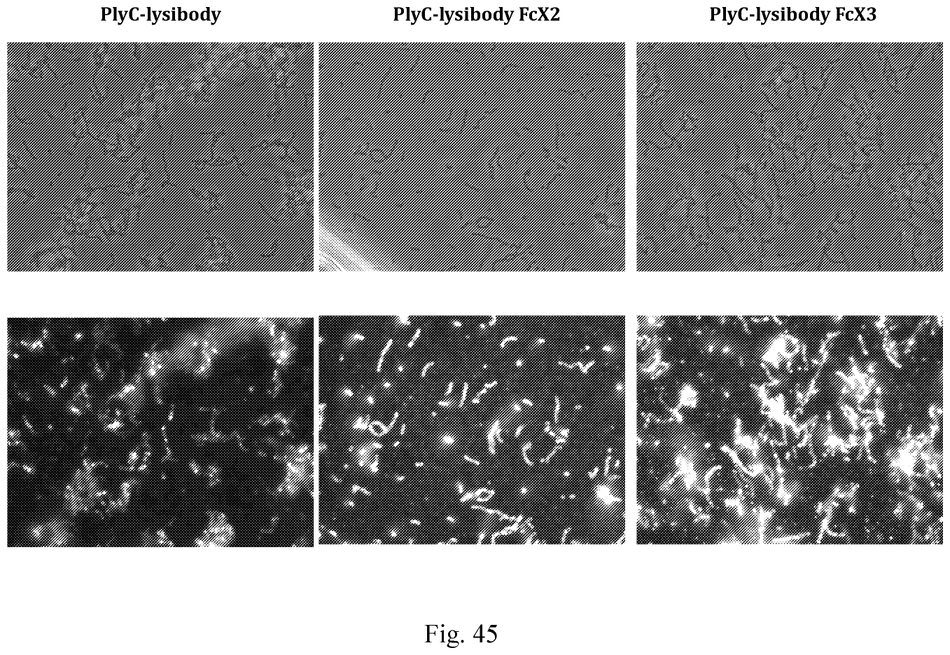

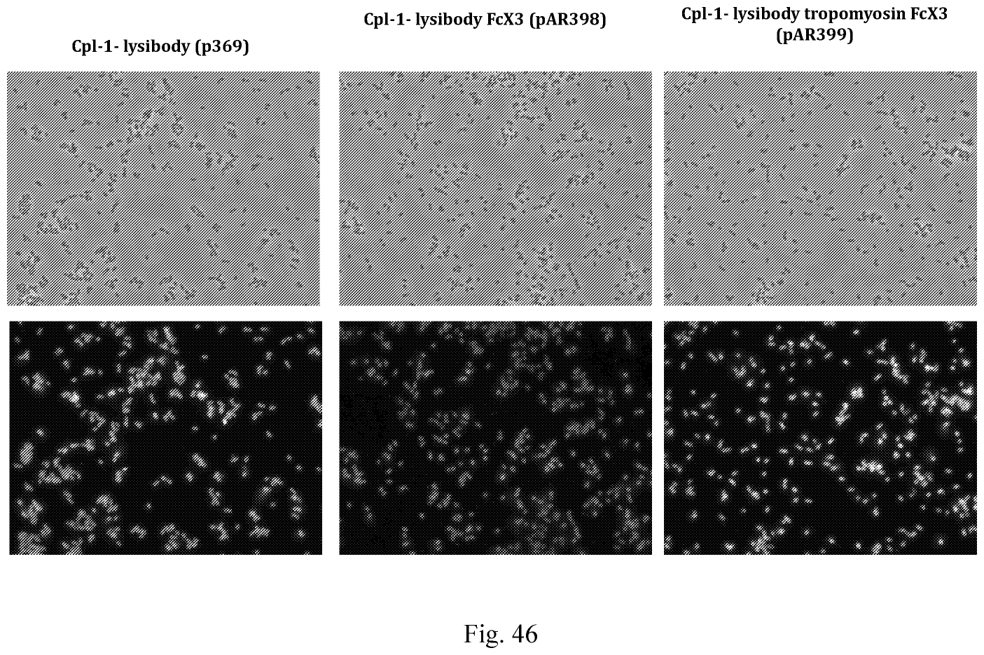

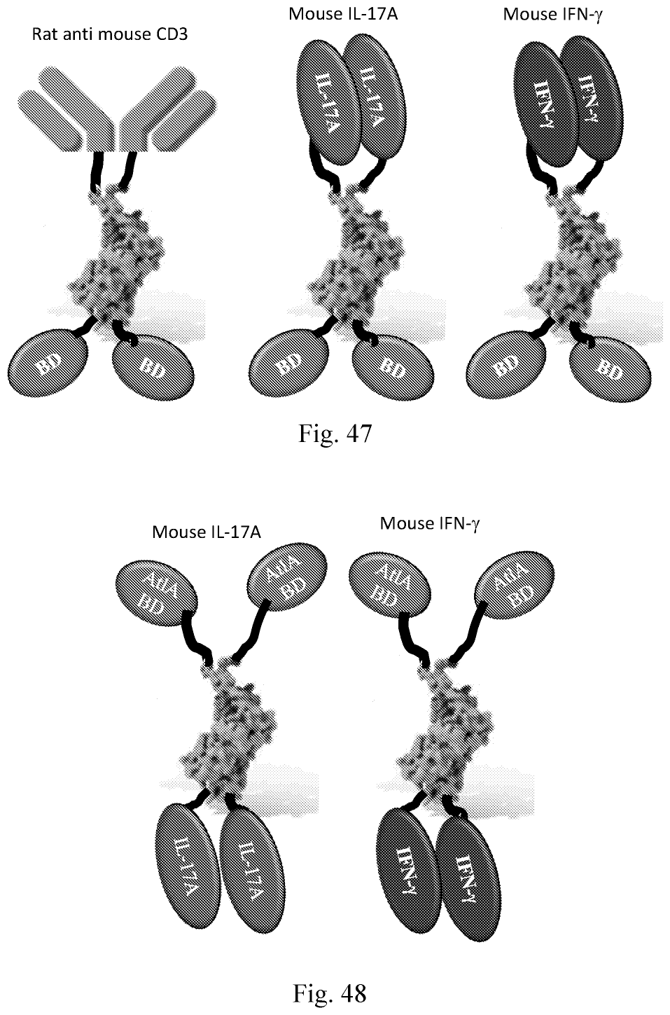

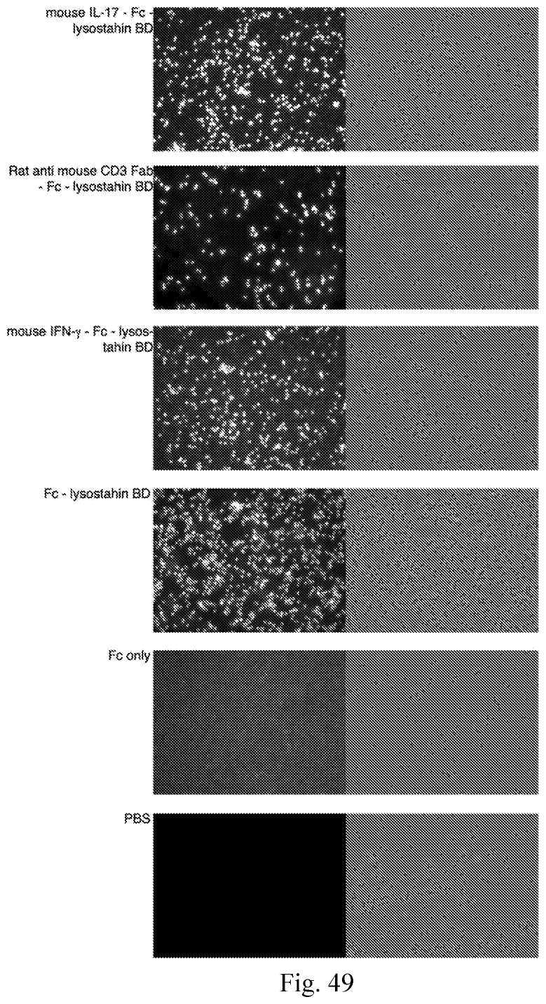

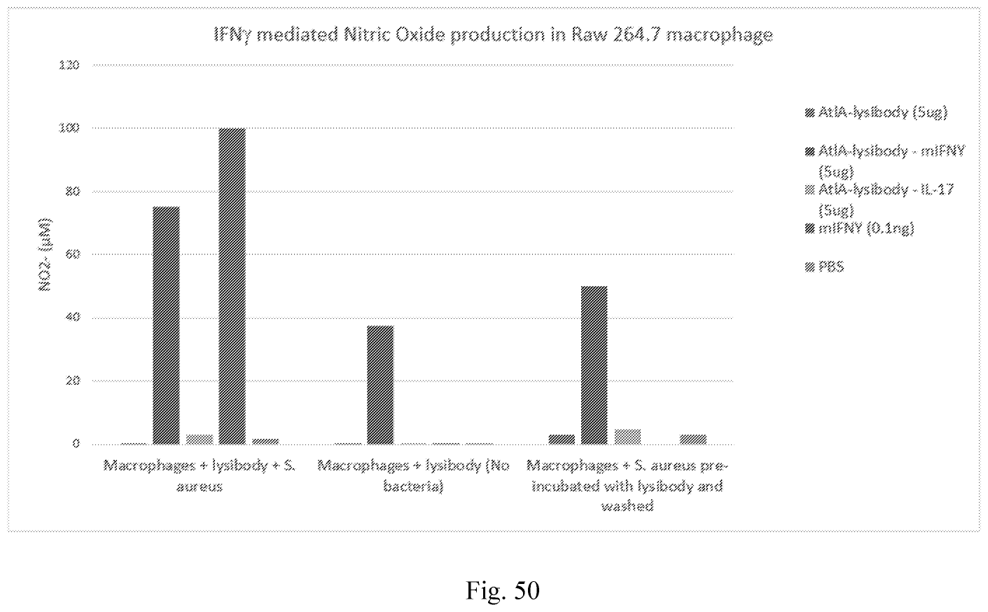

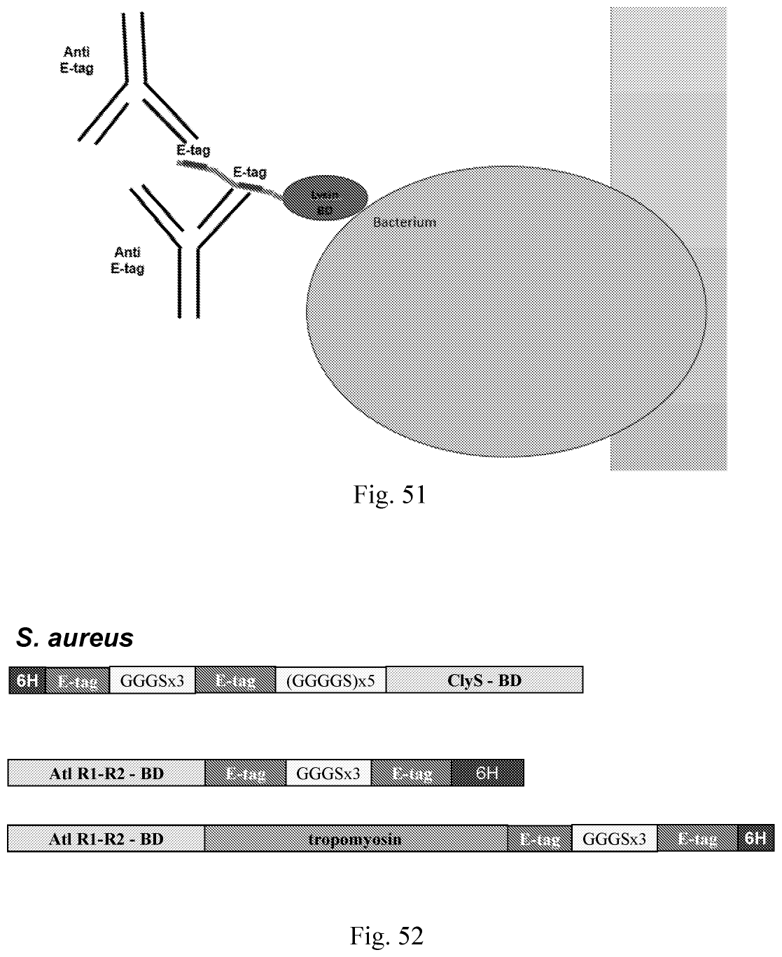

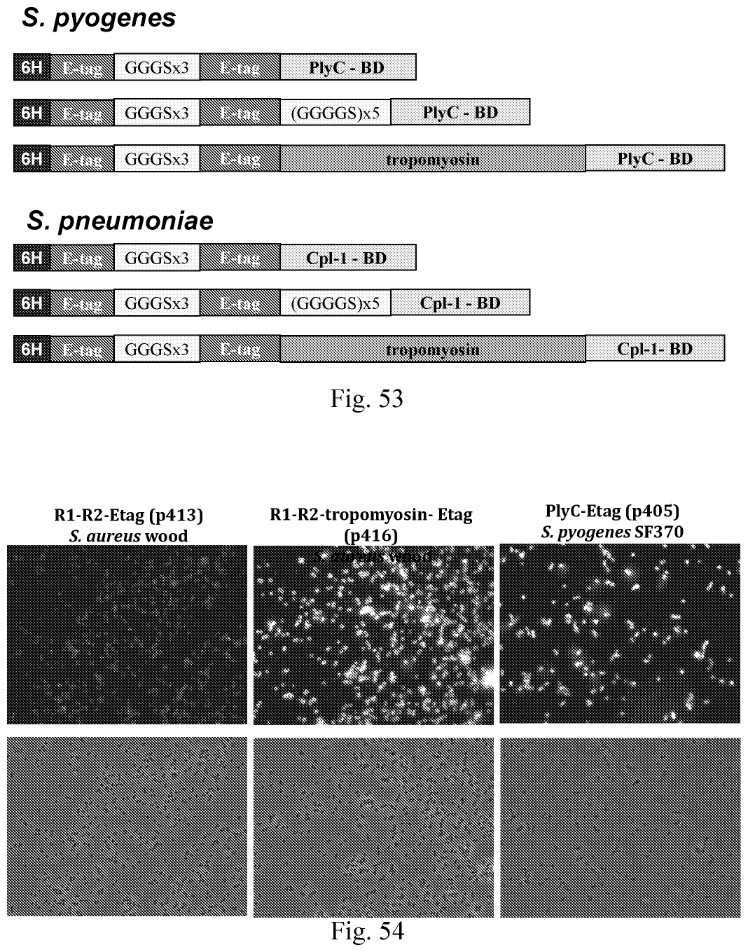

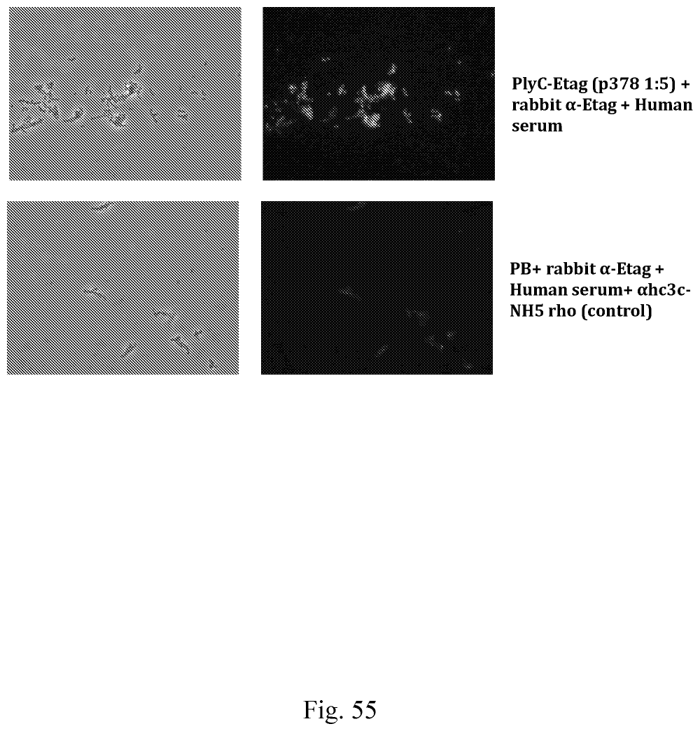

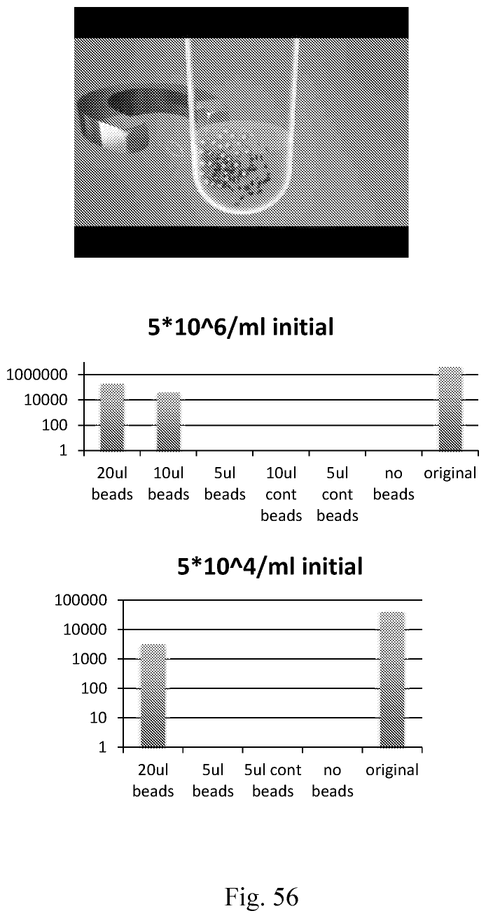

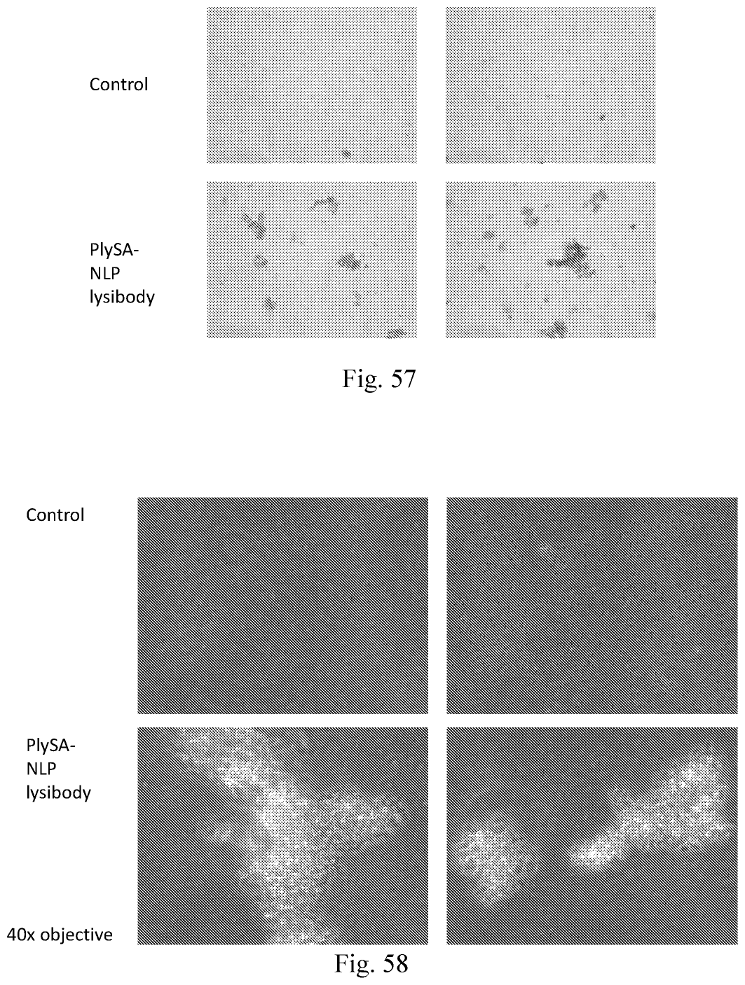

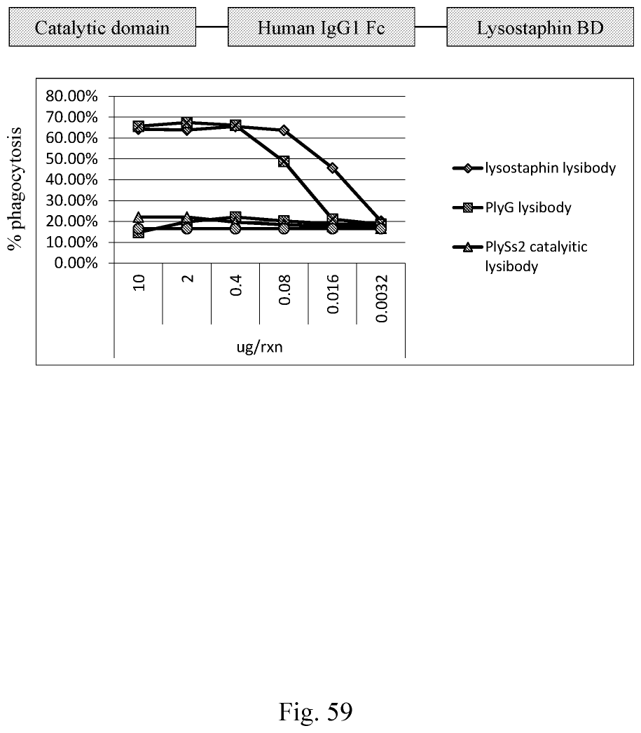

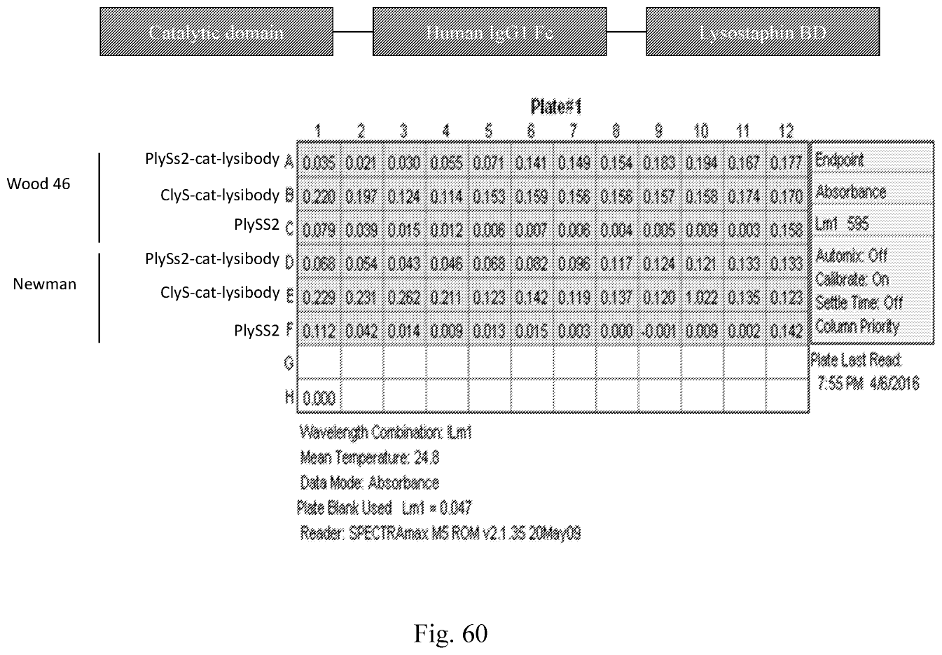

1. As supported by FIGS. 25-31, Lysibodies can be produced with a variety of different binding domains, and can target a variety of gram-positive organisms. For example, the inventors produced lysibodies using binding domains from the following lysin molecules:
   a. Staphylococcus aureus:
      i. AtlA (autolysin)
      ii. PlySs2
      iii. ClyS
      iv. Lysostaphin
      v. LysK
      vi. M23-ami-LysM
      vii. PlySA
      viii. Ply32
   b. Streptococcus pyogenes:
      i. PlyC
      ii. spy0077 SH3 (autolysin)
   c. Streptococcus pneumoniae:
      i. Cpl-1
      ii. PAL
   d. Bacillus anthracis:
      i. PlyG 2. As supported by FIGS. 32-35, Lysibodies can be produced with any type of IgG Fc.
Non-limiting examples of lysibodies produced by the inventors that contain different Fc regions include:
   a. ClyS-lysibody with human IgG3.
   b. AtlA, ClyS, PlySs2 lysibodies with mouse IgG2a.
   c. AtlA-lysibody with mouse IgG1.
3. As shown in one non-limiting example that is supported by FIG. 36, Lysibodies can be created with an Fc region that is mutated to enhance or diminish specific effector functions. Non-limiting examples of such lysibodies produced by the inventors include:
   a. AtlA-lysibody with human IgG1 Fc containing the following mutations:
      i. G236A S239D
      ii. L328R
   b. PlySs2-lysibody with human IgG1 Fc containing the following mutations:
      i. G236A S239D I332E
      ii. G236A S239D A330L I1332E
   c. ClyS-lysibody with human IgG1 Fc containing the following mutations:
      i. G236A S239D A330L I332E
   d. Lysostaphin-lysibody with human IgG1 Fc containing the following mutations:
      i. G236A S239D A330L I332E
   e. LysK-lysibody with human IgG1 Fc containing the following mutations:
      i. G236A S239D A330L I332E
   f. PlyC-lysibody with human IgG1 Fc containing the following mutations:
      i. G236A S239D A330L I332E
4. As supported by FIGS. 37-42, Lysibodies can be produced with linkers (flexible, rigid, or extended) between the binding domain and the Fc region. Linkers can serve to enhance lysibody activity, to prevent steric constraints, and to extend size and reach of the lysibody. Specifically, extended linkers could provide a lysibody with reach to extend beyond certain anti-phagocytic capsules, allowing recognition by phagocytes. Non-limiting examples of such lysibodies produced by the inventors include:
   a. AtlA lysibody with:
      i. A flexible glycine-serine linker.
      ii. Tropomyosin linker (extended coiled-coil)
   b. PlySs2 lysibody with:
      i. A flexible glycine-serine linker.
      ii. Tropomyosin linker (extended coiled-coil)
   c. ClyS lysibody with:
      i. A flexible glycine-serine linker.
      ii. Tropomyosin linker (extended coiled-coil)
   d. Cpl-1 lysibody with:
      i. A flexible glycine-serine linker.
      ii. Tropomyosin linker (extended coiled-coil)
   e. PAL lysibody with:
      i. A flexible glycine-serine linker.
      ii. Tropomyosin linker (extended coiled-coil)
   f. PlyC lysibody with:
      i. A flexible glycine-serine linker.
      ii. Tropomyosin linker (extended coiled-coil)
      iii. A double tropomyosin linker (highly extended coiled-coil)
   g. PlyG lysibody with:
      i. A flexible glycine-serine linker.
      ii. Tropomyosin linker (extended coiled-coil)
5. As supported by FIGS. 43-46, Lysibodies can be fused to multiple Fc regions, which will lead to an enhancement of effector function per lysibody. Non-limiting examples of such lysibodies produced by the inventors include:
   a. PlySs2 lysibody with:
      i. Two Fc molecules.
      ii. Three Fc molecules.
   b. ClyS lysibody with:
      i. Three Fc molecules.
   c. PlyC lysibody with:
      i. Two Fc molecules.
      ii. Three Fc molecules.
   d. Cpl-1 lysibody with:
      i. Two Fc molecules.
      ii. Three Fc molecules.
6. As supported by FIGS. 47-50, Lysibodies can be fused to other effector molecules such as cytokines to enhance effector functions, including but not limited to:
   a. AtlA-lysibody fused to (C-terminal fusion):
      i. Interferon gamma.
      ii. IL-17.
   b. Lysostaphin-lysibody fused to (N-terminal fusion):
      i. Interferon gamma.
      ii. IL-17.
7. As supported by FIGS. 51-55, Lysin binding domains could be fused to the C-terminus of the heavy chain of an intact lysibody, or to a single-chain antibody to create a bi-specific antibody. This can be used to target multiple organisms or to target effector cells to produce a desired effect. Non-limiting examples of such lysibodies the inventors produced include:
   a. Lysostaphin-BD fusion to the C-terminus of KT3 antibody heavy chain (targeting the CD3 molecule of T-cells). This molecule is co-expressed with a plasmid expressing the KT3 light chain to produce the mature molecule.
8. Lysibodies can be produced to contain more than one binding domain through strategies such as:
   a. Fusion of different binding domains at the N and C terminus of the molecule to produce a bi-specific lysibody.
   b. Through a knobs-into-holes strategy of Fc engineering.
9. As supported by FIG. 56, Lysin binding domains could be fused to an immunogenic tag such as E-tag, and introduced into an individual with pre-existing immunity to this tag to direct immunity to the target organism. Alternatively anti-tag antibody can be co-injected to the individual. This approach could be combined with the linker approach. Non-limiting examples of such lysibodies produced by the inventors include:
   a. Double E-tag AtlA
   b. Double E-tag tropomyosin AtlA
   c. Double E-tag PlyC
   d. Double E-tag—flexible linker—PlyC
   e. Double E-tag—tropomyosin—PlyC
   f. Double E-tag Cpl-1
   g. Double E-tag—flexible linker—Cpl-1
   h. Double E-tag—tropomyosin—Cpl-1
10. As supported by FIGS. 57 and 58, Lysibodies can be used to isolate bacteria from clinical specimens using magnetic beads and other techniques. For example:
   a. Lysibodies can be attached to protein A magnetic beads and incubated with blood from a patient, and subsequently isolated using a magnet.
   b. Blood can be cultured prior to isolation to increase sensitivity.
   c. Blood can be lysed with detergents and spun down prior to isolation to increase sensitivity.
11. Lysibodies can be used in the identification of bacteria through a number of methods such as an agglutination test. For example:
   a. A suspension of target organisms could be suspended in buffer and lysibody added. Aggregation would indicate the presence of an organism recognized by the binding domain.
   b. Similarly latex beads with immobilized lysibody could be used to increase sensitivity.
12. As supported by FIGS. 59 and 60, Lysibodies with both catalytic and binding domains could be created to provide improved pharmacokinetics compared to a native lysin molecule:
   a. A catalytic domain could be fused to the free side of the Fc molecule in a lysibody to mediate killing of the target organisms.
   b. An intact lysin molecule could be fused to the Fc region of a human antibody.

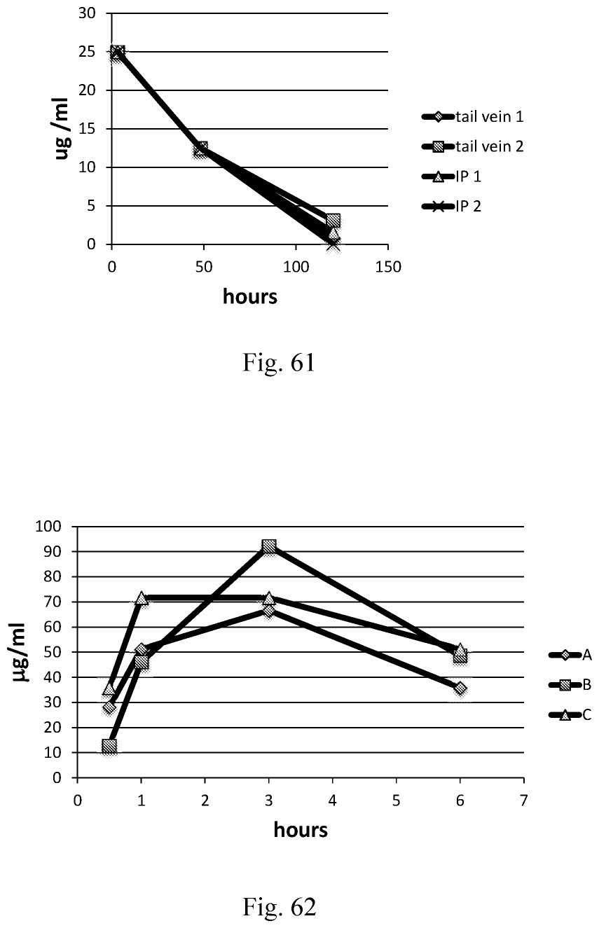

The pharmacokinetics of representative and non-limiting Lysibodies of this disclosure are illustrated by FIGS. 61 and 62.

It will be apparent from the foregoing that this disclosure describes the development of a novel solution to a long-standing problem in immunology—how to create high-affinity opsonic antibodies to invariant bacterial cell wall carbohydrates. We demonstrate that binding domains from cell wall hydrolases direct the Fc portion of an antibody to bacterial wall carbohydrates, and that the fusion protein function like a normal antibody: efficiently binding, opsonizing, inducing complement fixation, promoting phagocytosis of bacteria by macrophages and neutrophils, and protecting animals in infectious models. We produced different lysibodies specific for S. aureus, using binding domains from the major staphylococcal autolysin AtlA, as well as two phage lysins—ClyS and PlySs2.

Without intending to be constrained by any particular theory, it is considered that a major advantage of lysibodies compared to typical monoclonal antibodies is their ability to bind abundant carbohydrate targets on the bacterial wall, which are highly conserved and thus unlikely to mutate to avoid binding. Many surface carbohydrates are critical for proper cell wall function. In S. aureus the membrane bound carbohydrate lipoteichoic acid (LTA) is essential, and while mutants lacking wall teichoic acid (WTA) are viable, they have impaired pathogenicity and are less able to colonize the host. When using a binding domain derived from the pathogen's own autolysin, as in the case of the AtlA-lysibody, mutations that would prevent lysibody binding would necessarily also disturb the action of the native autolysin, interfering with cell division. Resistance to lysibodies containing a binding domain from a phage lysin is also unlikely; lysins have evolved over a billion years to bind wall targets that cannot easily mutate, since a phage that does not produce a functional lysin would be trapped inside the infected host, and thus lost from the population. Supporting this, resistance to phage lysins was not observed following selection procedures that readily produce antibiotic resistance. Another advantage of targeting wall carbohydrates is that they are often conserved across species, resulting in a broad range of target organisms. For example, AtlA-lysibody and ClyS-lysibody bound all strains of S. aureus tested, as well as several coagulase-negative staphylococci, while PlySs2-lysibody bound streptococci and enterococci in addition to staphylococci. The protein targets of monoclonal antibodies on the other hand, are often variable even at the species level and may not always be expressed, potentially allowing variants to escape treatment.

The approaches described herein open a new avenue for the development of therapeutic antibodies, using binding domains that were optimized through evolution. Lysibodies could be produced for a range of additional Gram-positive pathogenic bacteria, given the wealth of autolysins and phage lysins found in nature. Furthermore, data presented herein suggest that other proteins with high affinity binding to a surface receptor on a pathogen may similarly be modified with an Fc to produce a functional opsonic antibody. Thus, lysibodies represent a new class of anti-infectives that resolve the long-standing problem of effectively targeting bacterial surface carbohydrates with antibodies.

While the invention has been particularly shown and described with reference to specific embodiments (some of which are preferred embodiments), it should be understood by those having skill in the art that various changes in form and detail may be made therein without departing from the spirit and scope of the present invention as disclosed herein.

1. A polypeptide comprising at least one immunoglobulin fragment crystallizable region (Fc) segment and at least one additional segment that comprises a binding domain that binds with specificity to a component of a bacterial cell wall, wherein the binding domain is a binding domain from: a bacterial autolysin, a bacteriophage lysin, a bacteriophage tail or tail fiber, a bacteriocin, or a combination thereof.

2. The polypeptide of claim 1, wherein the Fc segment comprises a CH2 and CH3 of the Fc region, and optionally comprises an Fc CH1 region.

3. The polypeptide of claim 1, wherein the polypeptide comprises the binding domain of a bacterial autolysin.

4. The polypeptide of claim 1, wherein the polypeptide comprises the binding domain of a phage lysin.

5. The polypeptide of claim 1, wherein the polypeptide comprises the binding domain of a bacteriophage tail or tail fiber.

6. The polypeptide of claim 1, wherein the polypeptide comprises the binding domain of a bacteriocin.

7. The polypeptide of claim 1, further comprising at least one additional Fc region.

8. The polypeptide of claim 1, wherein the binding domain is N-terminal in the polypeptide relative to the Fc segment.

9. The polypeptide of claim 1, wherein the binding domain is C-terminal in the polypeptide relative to the Fc segment.

10. The polypeptide of claim 1, wherein the polypeptide is reversibly or irreversibly attached to a substrate.

11. The polypeptide of claim 10, wherein the polypeptide is in physical association with a molecule on a surface of a bacteria via the binding domain.

12. The polypeptides of claim 10, wherein the polypeptide comprises a linker between the Fc and the binding domain.

13. A DNA polynucleotide encoding a polypeptide of claim 1.

14. The DNA polynucleotide of claim 13, wherein the DNA polynucleotide is present in an expression vector.

15. Cells comprising a DNA polynucleotide of claim 14.

16. A method of inhibiting growth of bacteria and/or killing bacteria or a parasite in a population of bacteria or parasites comprising contact the bacteria or the parasites in the population with a polypeptide of claim 1.

17. The method of claim 16, wherein the bacteria comprise pathogenic bacteria that are resistant to one or more antibiotics.

18. The method of claim 16, wherein the bacteria are in or on an individual in need of treatment for an infection by the bacteria.

19. The method of claim 18, wherein the individual is at risk of contracting an infection caused by the bacteria.

20. The method of claim 16, wherein the bacteria are present on a mucosal surface.

21. A method of making a polypeptide of claim 1 comprising allowing expression of the polypeptide in a population of mammalian cells comprising an expression vector encoding the polypeptide, and separating the polypeptide from the population of cells after the expression.

22. An article of manufacture comprising a polypeptide of claim 1, the article comprising a container comprising the polypeptide, the article further comprising printed material providing an indication that the polypeptide is used for killing and/or inhibiting the growth of bacteria.

23. A library of polypeptides of claim 1, wherein the library comprises a plurality of polypeptides that each have a distinct binding domain.

24. A method for treating an individual in need thereof comprising testing a sample from the individual for the presence of a bacterial infection, determining the presence of the bacteria, and selecting a polypeptide of claim 1, wherein the selected polypeptide has a binding domain that is specific for the cell wall of the determined bacteria, and contacting the bacteria with the selected polypeptide.

25. The method of claim 24, wherein the polypeptide is a member of a library comprising a plurality of polypeptides that each have a distinct binding domain.

26. A kit comprising a polypeptide of claim 1.


SEQUENCE LISTING (34 sequences)

SEQ ID NO: 1 - protein, 121 aa, artificial sequence
  Bacteria surface ligand binding domain
STAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 2 - protein, 100 aa, artificial sequence
  Bacterial sequence binding domain as part of chimeric protein
TPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 3 - protein, 129 aa, artificial sequence
  Bacterial binding domain as part of a chimeric protein
MNKITNKVKPPNRDGINKDKIVYDRTNINYNMVLQGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 4 - protein, 352 aa, artificial sequence
  Bacterial binding domain as part of a chimeric protein
TTTPTTPSKPTTPSKPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKPGTKLYTVPWGTSKQVAGSVSGSGNQTFKASKQQQIDKSIYLYGSVNGKSGWVSKAYLVDTAKPTPTPTPKPSTPTTNNKLTVSSLNGVAQINAKNNGLFTTVYDKTGKPTKEVQKTFAVTKEASLGGNKFYLVKDYNSPTLIGWVKQGDVIYNNAKSPVNVMQTYTVKPGTKLYSVPWGTYKQEAGAVSGTGNQTFKATKQQQIDKSIYLFGTVNGKSGWVSKAYLAVPAAPKKAVAQPKTA

SEQ ID NO: 5 - protein, 131 aa, artificial sequence
  Bacterial binding domain as part of a chimeric protein
KQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 6 - protein, 19 aa, artificial sequence
  Segment of chimeric protein
TGHHHHHHGGGGSGGGSGR

SEQ ID NO: 7 - DNA, 47 bp, artificial sequence
  Primer
ccggtagcggccgcctgcagggatcctctagagatatccagctgaag

SEQ ID NO: 8 - DNA, 47 bp, artificial sequence
  Primer
tcgacttcagctggatatctctagaggatccctgcaggcggccgcta

SEQ ID NO: 9 - DNA, 50 bp, artificial sequence
  Primer
ccggtcatcatcatcatcatcatggaggaggaggaagcggaggaggaagc

SEQ ID NO: 10 - DNA, 50 bp, artificial sequence
  Primer
ggccgcttcctcctccgcttcctcctcctccatgatgatgatgatgatga

SEQ ID NO: 11 - DNA, 43 bp, artificial sequence
  Primer
cccgcggccgcatgacaactacccctactacaccatcaaaacc

SEQ ID NO: 12 - DNA, 33 bp, artificial sequence
  Primer
gggctgcagagctgtttttggttgtgctactgc

SEQ ID NO: 13 - DNA, 31 bp, artificial sequence
  Primer
cccctgcagcccaaatctggtgacaaaactc

SEQ ID NO: 14 - DNA, 29 bp, artificial sequence
  Primer
cttaagctttcatttacccggagacaggg

SEQ ID NO: 15 - DNA, 33 bp, artificial sequence
  Primer
caagcggccgccccaaatctggtgacaaaactc

SEQ ID NO: 16 - DNA, 28 bp, artificial sequence
  Primer
cacctgcagtttacccggagacagggag

SEQ ID NO: 17 - DNA, 49 bp, artificial sequence
  Primer
ggggggatccgggtctagaggaagatctggaggaggaggggatatcaag

SEQ ID NO: 18 - DNA, 57 bp, artificial sequence
  Primer
tcgacttgatatcccctcctcctccagatcttcctctagacccggatcccccctgca

SEQ ID NO: 19 - DNA, 42 bp, artificial sequence
  Primer
ccggtcgaccatgaataagatcacaaataaagttaaaccacc

SEQ ID NO: 20 - DNA, 39 bp, artificial sequence
  Primer
cccaagcttttaaaacacttctttcacaatcaatctctc

SEQ ID NO: 21 - DNA, 34 bp, artificial sequence
  Primer
gaggtcgaccacaccgcctggcacggtcgcacag

SEQ ID NO: 22 - DNA, 34 bp, artificial sequence
  Primer
gagaagcttttatttaaatgtaccccaagcattg

SEQ ID NO: 23 - DNA, 35 bp, artificial sequence
  Primer
ggggcggccgcatggccgaggtgcagctgttggag

SEQ ID NO: 24 - DNA, 36 bp, artificial sequence
  Primer
cccctgcagtcgtttgatttccaccttggtcccttg

SEQ ID NO: 25 - DNA, 30 bp, artificial sequence
  Primer
ggggtcgactcatgtggcgactacttcacc

SEQ ID NO: 26 - DNA, 33 bp, artificial sequence
  Primer
cccaagcttttatttaacttcataccaccaacc

SEQ ID NO: 27 - DNA, 33 bp, artificial sequence
  Primer
cccgcggccgccccaaatctggtgacaaaactc

SEQ ID NO: 28 - DNA, 41 bp, artificial sequence
  Primer
ccgtctagaatgaataagatcacaaataaagttaaaccacc

SEQ ID NO: 29 - DNA, 39 bp, artificial sequence
  Primer
gcgctgcagttaaaacacttctttcacaatcaatctctc

SEQ ID NO: 30 - DNA, 78 bp, artificial sequence
  Primer
cgcgaattcatgagtaaaggagaacttcatcatcatcatcatcattcctccgccatgagtaaaggagaagaacttttc

SEQ ID NO: 31 - DNA, 30 bp, artificial sequence
  Primer
gagggtacctttgtatagttcatccatgcc

SEQ ID NO: 32 - DNA, 33 bp, artificial sequence
  Primer
gagtctagaacaccgcctggcacggtcgcacag

SEQ ID NO: 33 - DNA, 34 bp, artificial sequence
  Primer
gggctgcagttatttaaatgtaccccaagcattg

SEQ ID NO: 34 - DNA, 38 bp, artificial sequence
  Primer
cgcctgcagttatttgtatagttcatccatgccatgtg